(19) United States
(12) Patent Application Publication
(10) Pub. No.: US 2024/0050596 A1
(43) Pub. Date: Feb. 15, 2024

(54) DYE AGGREGATES-CONTAINING NANOPARTICLES AND USES THEREOF

(71) Applicant: BOARD OF REGENTS, THE UNIVERSITY OF TEXAS SYSTEM, Austin, TX (US)

(72) Inventors: Keith P. Johnston, Austin, TX (US); Thomas Truskett, Austin, TX (US); Behzad Changalvaie, Woodland Hills, CA (US); Ehsan Moaseri, Austin, TX (US); Konstantin V. Sokolov, Missouri City, TX (US); Sangheon Han, Houston, TX (US); Mohammed Kawelah, Austin, TX (US)

(21) Appl. No.: 18/487,550

(22) Filed: Oct. 16, 2023

Related U.S. Application Data
(63) Continuation of application No. 17/104,859, filed on Nov. 25, 2020.
(60) Provisional application No. 62/941,158, filed on Nov. 27, 2019.

(51) Int. Cl. A61K 49/22; C09B 67/02; C09B 23/01; A61K 47/34; A61K 49/04; A61K 49/00; A61K 49/18

(52) U.S. Cl. CPC A61K 49/225; C09B 67/0097; C09B 23/0075; A61K 47/34; A61K 49/04; A61K 49/0093; A61K 49/0002; A61K 49/1824

(57) ABSTRACT

The present invention provides compositions relating to nanoparticles, such as nanocapsules, that selectively target cells associated with diseases or disorders (e.g., cancer cells). The present invention further relates to methods relating to the said nanoparticles for imaging, detection, and treatment of diseases or disorders in a subject. The present invention additionally provides kits that find use in the practice of the methods of the invention.

### CROSS-REFERENCE TO RELATED APPLICATIONS

This application claims priority to and the benefit of U.S. Provisional Application No. 62/941,158, filed Nov. 27, 2019, the disclosure of which is incorporated herein by reference in its entirety.

### BACKGROUND OF THE INVENTION

Photoacoustic imaging (PAI) is an emerging modality that has attracted attention as a platform for molecular and functional tissue imaging with a penetration depth in tissue of up to a few centimeters (Bouchard R et al., 2014, IEEE Trans. Ultrason. Ferroelectr. Freq. Control, 61:450-466; Weber J et al., 2016, Nat. Methods, 13:639-650). In PAI, irradiation of tissue by a pulsed laser generates acoustic waves in areas with optical absorption due to the photoacoustic (PA) effect. The acoustic waves are detected by an ultrasound transducer whereby the PA signal intensity is proportional to the local laser fluence and tissue absorption. Near-infrared (NIR) lasers are usually used to achieve high tissue penetration. The strongest endogenous contrasts in PAI are produced by hemoglobin and melanin that have been used to image vascular structures (Zhang H F et al., 2006, Nature Biotechnology, 24:848-851), tumor angiogenesis (Zhang H F et al., 2006, Nature Biotechnology, 24:848-851; Diot G et al., 2017, Clin. Cancer Res., 23:6912-6922), and internal organs (Song K H et al., 2007, Journal of Biomedical Optics, 12: 060503) in vivo. To extend molecular imaging capabilities of PAI, significant efforts have been focused on development of exogenous contrast agents, including various inorganic nanoparticles (NPs), such as noble-metal-based nanostructures (Mallidi S et al., 2007, Opt. Express, 15:6583-6588; Luke G P et al., 2014, Cancer Res., 74:5397-5408; Fu Q et al., 2019, Adv. Mater., 31: 1805875; Chen Y S et a., 2019, Nat. Nanotechnol., 14:465-472; Bayer C L et al., 2013, Journal of Biomedical Optics, 18: 016001), carbon nanomaterials (Jin Y et al., 2013, Biomaterials, 34:4794-4802; de la Zerda A et al., 2010, Nano Lett., 10:2168-2172), and semiconductor nanoparticles (Cheng L et al., 2014, Advanced Materials, 26:1886-1893). However, clinical translation of inorganic nanomaterials is limited due to their non-degradability (Yang K et al., 2013, Small, 9:1492-1503; Khlebtsov N et al., 2011, Chemical Society Reviews, 40:1647-1671; Hao J et al., 2017, Advanced Science, 4: 1600160) and potential long-term toxicity in vivo (Muthu M S et al., 2014, Theranostics, 4:660-677; Wang J et al., 2016, Journal of Controlled Release, 237:23-34). Therefore, there has been a shift towards development of fully organic NPs as contrast agents for PAI (Xu L et al., 2014, Polymer Chemistry, 5:1573-1580; Yoon H K et al., 2013, J. Mater. Chem. B, 1:5611-5619; Zhang Y et al., 2014, Nat. Nanotechnol., 9:631-638; Lu H D et al., 2017, ACS Comb. Sci., 19:397-406; Harmatys K M et al., 2018, Angew. Chem. Int. Ed. Engl., 57:8125-8129) with improved biodegradation and body clearance, for example, via encapsulation in micelles (Lu H D et al., 2017, ACS Comb. Sci., 19:397-406).

Amongst organic chromophores, indocyanine green (ICG) is currently the only NIR-absorbing dye approved by the U.S. Food and Drug Administration (FDA) (Liu R et al., 2017, Nanotheranostics, 1:430-439). ICG, an amphiphilic tricarbocyanine dye, is mainly used in sentinel lymph node biopsy and during surgery evaluation of blood flow (Akman L et al., 2016, Journal of Radioanalytical and Nuclear Chemistry, 308:659-670; Kuo W-S et al., 2012, Biomaterials, 33:3270-3278). Despite its clinical use, ICG suffers from a short blood circulation time (half-life of 2-4 min) (Taichman G C et al., 1987, Texas Heart Institute Journal, 14:133-138; Desmettre T et al., 2000, Survey of Ophthalmology, 45:15-27) because of its propensity to bind to serum albumins and then undergo rapid clearance by the liver (Dinsmore A et al., 2002, Science, 298:1006-1009; Wolfe J D et al., 2004, Experimental Eye Research, 79:631-638). Extensive studies have enhanced ICG circulation time with several clinically-applicable delivery systems including liposomes (Yan F et al., 2016, Journal of Controlled Release, 224:217-228), polymers (Ma Y et al., 2013, Biomaterials, 34:7706-7714), lipid NPs (Zheng M et al., 2013, ACS Nano, 7:2056-2067; Navarro F P et al., 2012, Journal of Biomedical Nanotechnology, 8:730-741; Zheng C et al., 2012, Biomaterials, 33:5603-5609; Zhao P et al., 2014, Biomaterials, Wang T et al., 2010, Molecular Pharmaceutics, 7:1007-1014), modified silicate matrices (Lee C H et al., 2009, Advanced Functional Materials, 19:215-222), and albumin nanoparticles (Sheng Z et al., 2014, ACS Nano, 8:12310-12322). ICG has also been loaded into antibody coated nanocapsules of salt cross-linked polyallylamine for photothermal therapy in the visible region (Yu J et al., 2010, Journal of the American Chemical Society, 132:1929-1938). Although these strategies are promising in addressing ICG pharmacokinetics, they do not address photothermal degradation and poor aqueous solubility of ICG dye that limit its applications as a contrast agent for PAI (Sheng Z et al., 2013, Nano-Micro Letters, 5:145-150).

Recent reports indicate that ICG J aggregates (ICGJ) exhibit significantly improved photothermal stability as compared to monomeric ICG24, a requirement for the development of PAI agents. At concentrations >10⁻³ M in aqueous solution, ICG can self-assemble in a few days at elevated temperatures (usually at 65° C.) into J-type aggregates (ICGJ) characterized by a narrow red-shifted absorbance peak at 890 nm (Liu R et al., 2017, Nanotheranostics, 1:430-439; Rotermund F et al., 1997, Chemical Physics, 220:385-392; Mauerer M et al., 1998, Journal of Photochemistry and Photobiology B: Biology, 47:68-73; Wittmann M et al., 1998, Applied Physics B: Lasers and Optics, 66:453-459), a feature that arises due to delocalization of ICG's excitations to form Frenkel excitons in the self-organized structures (Zweck J et al., 2001, Chemical Physics, 269:399-409). While ICGJ has distinct advantages compared to ICG monomer, such as a sharp NIR peak and greater photostability, it is not stable in biological environments as it dissociates in serum on the order of minutes. Therefore, development of encapsulation strategies to stabilize ICGJ is critical for applications in biology and medicine.

Previously, J aggregates of bacteriopheophorbide-lipid (Bchl-lipid) with a strong extinction in the NIR have been stabilized successfully inside liposomes with rigid bilayers, but were found to dissociate with more flexible bilayers (Shakiba M et al., 2016, Nanoscale, 8:12618-12625). Furthermore, this strategy of encapsulating organic dyes in liposomal bilayers typically requires synthesis of dye-conjugated phospholipids. Relative to liposomes, polymer vesicles, such as polymersomes (Ps) comprising diblock copolymers, have thicker bilayer membranes, which may be beneficial for stabilizing J aggregates. Furthermore, Ps containing polyethylene glycol (PEG) provide resistance to protein opsonization (Discher B M et al., 1999, Science, 284:1143-1146; Jain S et al., 2003, Science, 300:460-464; Jang W S et al., 2015, Macromol. Rapid Commun., 36:378-384; Won Y Y et al., 2002, Journal of Physical Chemistry B, 106:3354-3364; Won Y Y et al., 1999, Science, 283:960-963). Ps may be produced via double water/oil/water (W/O/W) emulsions as the intermediate oil phase evaporates to achieve high loadings of hydrophilic cargoes (Blanazs A et al., 2009, Macromol. Rapid Commun., 30:267-277; Hayward R C et al., 2006, Langmuir, 22:4457-4461; Shum H C et al., 2011, Journal of the American Chemical Society, 133:4420-4426). During this process, the Ps bilayer membrane is formed as the inner and outer water domains coalesce with each other (Shum H C et al., 2011, Journal of the American Chemical Society, 133:4420-4426). The formation of a curved bilayer in Ps is favored when the weight fraction of a hydrophilic block ranges from −0.1 to 0.4 (Won Y Y et al., 2002, Journal of Physical Chemistry B, 106:3354-3364; Won Y Y et al., 1999, Science, 283:960-963) particularly for PEG (Jain S et al., 2003, Science, 300:460-464; Kim H O et al., 2013, Macromol. Biosci., 13:745-754; Oltra N S et al., 2014, Annual Review of Chemical and Biomolecular Engineering, Annual Reviews: Palo Alto, 5:281-299; Yu Y et al., 2012, Pharmaceutical Research, 29:83-96).

Among various types of nanocapsules, Ps have been studied extensively for a wide range of applications including NIR optical imaging (Ghoroghchian P P et al., 2005, Proc. Natl. Acad. Sci. U.S.A., 102:2922-2927; Leong J et al., 2018, Adv. Healthc. Mater., 7:27-31), for example, with encapsulated porphyrins, drug delivery (Oltra N S et al., 2014, Annual Review of Chemical and Biomolecular Engineering, Annual Reviews: Palo Alto, 5:281-299; Yu Y et al., 2012, Pharmaceutical Research, 29:83-96; Leong J et al., 2018, Adv. Healthc. Mater., 7:27-31; Lee J S et al., 2012, Journal of Controlled Release, 161:473-483), and immunization with an encapsulated toll-like receptor agonist (Dowling D J et al., 2017, J. Allergy Clin. Immunol., 140:1339-1350).

Thus, there is a need in the art for improved methods of imaging, detecting, and treating diseases or disorders, such as cancers, in a subject using selective targeting of cells associated with said diseases or disorders. The present invention satisfies this unmet need.

### BRIEF SUMMARY OF THE INVENTION

In one aspect, the present invention relates, in part, to a composition comprising a dye and a polymer nanocapsule encapsulating the dye. In one embodiment, the polymer nanocapsule is a polymer vesicle. In one embodiment, the polymer vesicle is a polymersome.

In some embodiments, the polymer nanocapsule has an average hydrodynamic diameter of about 50 nm to about 200 nm. In some embodiments, the dye is a dye aggregate that has an average hydrodynamic diameter of about 40 nm to about 60 nm. In some embodiments, the polymer nanocapsule has an absorption peak from about 680 to about 1100 nm wavelength. In some embodiments, the polymer nanocapsule has an absorption peak from about 780 to about 895 nm wavelength. In some embodiments, the polymer nanocapsule has an absorption peak from about 790 to about 895 nm wavelength. In some embodiments, the polymer nanocapsule has an absorption peak from about 785 to about 890 nm wavelength.

In some embodiments, the dye is a polymethine dye, cyanine dye, hemicyanine dye, streptocyanine dye, mercocyanine dye, oxonol dye, styryl dye, diarylmethine dye, triarylmethine dye, rylene, squaraine, perylene bismide dye or aza-analog thereof, indocyanine green (ICG), Congo Red, IR783, Briliant Blue G, rhodamine 6G, or any combination thereof.

In one aspect of the invention, the polymer nanocapsule comprises a block copolymer. In some embodiments, the block copolymer is poly(ethylene oxide) (PEO) block copolymer, poly(lactide-co-glycolic acid)-block-polyethylene glycol (PLGA-b-PEG), a biodegradable PLGA-b-PEG, poly(ethylene oxide)-block-poly(ε-caprolactone) (PEO-b-PCL), polyanhydride-block-PEG copolymers, zwitterionic poly(carbobetaine) and zwitterionic poly(sulfobetaine)-containing block copolymers, poly(trimethylene carbonate)-block-poly(L-gluatamic acid), poly(ethylene glycol-block-L-aspartic acid), polypropylene oxide block copolymers, poly(ethylene oxide)-block-polypropylene oxide copolymers, or any combination thereof.

In one embodiment, the dye is an ICG aggregate and the polymer nanocapsule comprises a PLGA-b-PEG encapsulating the ICG aggregate. In one embodiment, the ICG aggregate is an indocyanine green J aggregate (ICGJ).

In one aspect of the invention, the composition further comprises a cationic polymer. In one embodiment, the polymer nanocapsule encapsulates the dye and the cationic polymer. In one embodiment, the cationic polymer is polyethyleneimine (PEI).

In some embodiments, the ratio of the dye to the cationic polymer is from about to 0.8.

In one aspect of the invention, the composition further comprises a targeting domain attached to the surface of the polymer nanocapsule. In one embodiment, the composition comprises a targeting domain attached to the surface of the polymer nanocapsule.

In one embodiment, the targeting domain binds to at least one cancer cell.

In some embodiments, the targeting domain is an antibody, an antibody fragment, a peptide sequence, aptamer, folate, ligand, a gene component, or any combination thereof. In some embodiments, the antibody is specific for at least one cancer biomarker. In one embodiment, the antibody is specific for epidermal-growth factor receptor (EGFR). In one embodiment, the antibody is a grafted monoclonal antibody.

In one embodiment, the polymer-encapsulated dye is conjugated with at least one antibody. In some embodiments, the polymer-encapsulated dye and the cationic polymer is conjugated with at least one antibody, an antibody fragment, nanobody, aptamer, or any combination thereof.

In one aspect of the invention, the composition further comprises a therapeutic agent. In some embodiments, the therapeutic agent is a hydrophilic therapeutic agent, hydrophobic therapeutic agent, or any combination thereof. In one embodiment, the hydrophobic therapeutic agent is encapsulated by the polymer nanocapsule or is complexed with the dye.

In some embodiments, the therapeutic agent is an antibiotic, antibody, small molecule, anti-cancer agent, chemotherapeutic agent, immunomodulatory agent, RNA molecule, siRNA molecule, DNA molecule, gene editing agent, gene-silencing agent, CRISPR-associated agent, guide RNA molecule, endonuclease, or any combination thereof.

In one embodiment, the therapeutic agent is on the exterior surface of the polymer nanocapsule. In one embodiment, the polymer nanocapsule encapsulates the therapeutic agent.

In another aspect, the present invention provides a contrast agent comprising at least one composition described herein and a pharmaceutically acceptable excipient. In one embodiment, the contrast agent is used in photoacoustic imaging (PAI).

In another aspect, the present invention provides an imaging method comprising contacting a biological tissue with at least one composition described herein; applying energy to a biological tissue comprising the composition; and imaging the biological tissue comprising the composition.

In one embodiment, the biological tissue is a cancer cell. In one embodiment, the cancer cell is present in a mammal.

In some embodiments, applying energy to the biological tissue comprises exposing the biological tissue to irradiation at a wavelength between about 680 and 1100 nm; irradiating at least a portion of the biological tissue with a light source; applying a radio frequency field; or any combination thereof.

In one embodiment, imaging the biological tissue comprises application of at least one imaging technique. In some embodiments, the imaging technique is photoacoustic imaging, ultrasound imaging, optical imaging, magnetic resonance imaging, computed tomography, thermal imaging, nuclear imaging, magnetomotive imaging enhancement, or any combination thereof.

In one embodiment, imaging the biological tissue comprises transducing the resulting ultrasound signal from the biological tissue and producing an image in a data processor from the transduced ultrasound signal.

In one embodiment, contacting the biological tissue with the composition results in release of the therapeutic agent from the polymer nanocapsule.

In one embodiment, applying energy to the biological tissue results in release of the therapeutic agent from the polymer nanocapsule.

In one aspect of the invention, the method further comprises allowing the composition to accumulate in a region of the biological tissue, wherein the targeting domain facilitated accumulation of the composition in the region.

In another aspect, the present invention provides a method of treating a disease or disorder in a subject in need thereof, the method comprising administering a therapeutically effective amount of at least one composition described herein to the subject.

### BRIEF DESCRIPTION OF THE DRAWINGS

The following detailed description of various embodiments of the invention will be better understood when read in conjunction with the appended drawings. For the purpose of illustrating the invention, there are shown in the drawings illustrative embodiments. It should be understood, however, that the invention is not limited to the precise arrangements and instrumentalities of the embodiments shown in the drawings.

FIG. 1, comprising FIG. 1A through FIG. 1D, depicts formation of ICG J aggregates (ICGJ). FIG. 1A depicts a schematic representation of ICGJ. FIG. 1B depicts the changes in extinction spectra of a 1.15 mg/mL ICG solution at 65° C. for 24 h: monomer (780 nm), dimer (715 nm) and J aggregate peak (˜890 nm) with isosbestic point. FIG. 1C depicts typical volume average diameters for ICGJ as determined via TEM (blue) and DLS (yellow). The volume average hydrodynamic diameter and standard deviation for 6 samples are given in Table 3 and Table 5. FIG. 1D depicts typical volume average diameters for ICGJ@PEI as determined via TEM (blue) and DLS (yellow). The volume average hydrodynamic diameter and standard deviation for 6 samples are given in Table 3 and Table 5.

FIG. 2, comprising FIG. 2A through FIG. 2C, depicts exemplary results of dissociation kinetics. While ICGJ and ICGJ@PEI fully dissociates in 12 min, the Polymersomes only experience a slight decrease in the J-aggregate peak at this time point. FIG. 2A depicts exemplary results of dissociation kinetics of 0.1 mg/mL ICGJ dispersed in 10% v/v ethanol/water solution. UV measurements were conducted after dilution at an ICG concentration of 0.01 mg/mL. FIG. 2B depicts exemplary results of dissociation kinetics of 0.1 mg/mL ICGJ@PEI NPs dispersed in 10% v/v ethanol/water solution. UV measurements were conducted after dilution at an ICG concentration of 0.01 mg/mL. FIG. 2C depicts exemplary results of dissociation kinetics of 0.1 mg/mL polymersomes dispersed in 10% v/v ethanol/water solution. UV measurements were conducted after dilution at an ICG concentration of 0.01 mg/mL. The tests were performed after already conducting the 6 separation steps shown in FIG. 33.

FIG. 3, comprising FIG. 3A through FIG. 3D, depicts zeta potential, stability, and evolution of volume average. FIG. 3A depicts zeta potential as a function of pH for PEI_{1.8k-branched}, ICG, ICGJ, ICGJ@PEI NPs, and polymersomes (Ps) or Ps conjugated with antibody (Ps-Ab). FIG. 3B depicts UV-vis-NIR spectra obtained after 3 hours exposure to chloroform-water interface indicating greater stability with added PEI (see also FIG. 4). FIG. 3C depicts exemplary UV-vis-NIR spectra before exposure. Representative spectra of ICG monomer, ICGJ, ICGJ@PEI NPs, and Ps loaded with ICGJ@PEI NPs at a concentration of mg/mL. FIG. 3D depicts evolution of the volume average D_{H} (left axis) of polymersomes (Ps) (blue) and the larger oil (O) droplets (red) during chloroform evaporation of W/O/W emulsion. The volume fraction of the polymersome peak (P) increases with time as shown by the black line with the label on the right y axis as the oil evaporates.

FIG. 4, comprising FIG. 4A through FIG. 4C, depicts exemplary results that demonstrate stability of ICGJ and ICGJ@PEI under mixing. FIG. 4A depicts UV-vis-NIR spectra of ICGJ after rapid stirring with 25 mg/mL PEG₁₀ₖ in DI water or with 25 mg/mL PLGA₉₀ₖ in chloroform. Upon mixing in water (no chloroform) with PEG the ICGJ aggregate peak did not change for 1 day. Upon mixing with PLGA in chloroform, ICGJ aggregates dissociated in <2 minutes, but ICGJ@PEI NPs degraded more slowly with complete degradation in two days. FIG. 4B depicts UV-vis-NIR spectra 24 hours after emulsification of 0.5 mL of 50 mg/mL ICGJ@PEI with both 1 mL chloroform and 1 mL 25 mg/mL PEG-PLGA in chloroform with intense probe sonication (5 cycles of 5 seconds at 30% intensity). FIG. 4C depicts UV-vis-NIR over the time of 0.5 mL of ICGJ 50 mg/mL were homogenized with 1 mL chloroform using an ultra-Turrax; the spectra are normalized to one at the 780 nm peak.

FIG. 5 depicts exemplary calibration curve of ICG absorbance at 790 nm wavelength vs. ICG concentration. The ICG was dissolved in 1/1 v/v ethanol/DI water solution.

FIG. 6 depicts zeta potential of ICGJ@PEI NPs with various amounts of PEI added to ICGJ. The “optimized PEI” refers to ICGJ@PEI NPs described in the experimental section and in FIG. 3. The small change at pH 4 with 2× the optimal amount (200 μL) suggests close to a monolayer was already present at the optimized level of 100 μL.

FIG. 7, comprising FIG. 7A through FIG. 7D, depicts exemplary results demonstrating stability and dissociation of 0.05 mg/mL ICGJ (FIG. 7A and FIG. 7B) and ICGJ@PEI (FIG. 7C and FIG. 7D) NPs in aqueous solution (DI) and 100% fetal bovine serum (FBS) at pH 5.5 and 7.4. FIG. 7A depicts exemplary results demonstrating stability and dissociation of 0.05 mg/mL ICGJ NPs in aqueous solution (DI) and 100% FBS at pH 5.5 and 7.4. FIG. 7B depicts exemplary results demonstrating stability and dissociation of 0.05 mg/mL ICGJ NPs in aqueous solution (DI) and 100% FBS at pH 5.5 and 7.4. FIG. 7C depicts exemplary results demonstrating stability and dissociation of 0.05 mg/mL ICGJ@PEI NPs in aqueous solution (DI) and 100% FBS at pH 5.5 and 7.4. FIG. 7D depicts exemplary results demonstrating stability and dissociation of 0.05 mg/mL ICGJ@PEI NPs in aqueous solution (DI) and 100% FBS at pH 5.5 and 7.4.

FIG. 8, comprising FIG. 8A through FIG. 8D, depicts a schematic illustration of the formation of polymersomes (ICGJ@PEI Ps-Ab). FIG. 8A depicts that a W₁/O emulsion is formed containing ICGJ@PEI within water droplets. FIG. 8B depicts that the oil phase is emulsified into water to form a W/O/W double emulsion. During evaporation, as the oil droplets shrink, the WE water droplets leave the oil phase and enter the outer water phase W₂ coated with a PEG-PLGA polymersome bilayer that encapsulates ICGJ@PEI NPs between the two arrows. FIG. 8C depicts that as the water droplets leave the oil phase, the yellow hydrophobic PLGA chains of the PEG-PLGA at the oil sides of the W1-O and O-W2 interfaces are attracted to each other and “zip” together to form the Ps bilayer wall with exterior hydrophilic PEG segments (red). FIG. 8D depicts that cross section of the resulting polymersomes shows the encapsulated ICGJ@PEI NPs surrounded by the PEG-PLGA bilayer. The antibodies are bonded to azide end-groups on the PEG segments via click chemistry.

FIG. 9 depicts UV-vis-NIR spectra of ICGJ@PEI polymersomes versus a mixture of ICGJ (83% wt) and ICG (17%). The peak intensities at 895 and 795 nm are in agreement with the calculated ICGJ to ICG ratio inside the polymersomes for the above weight ratio of 4.4.

FIG. 10, comprising FIG. 10A through FIG. 10D, depicts optimization of polymersome synthesis. FIG. 10A depicts UV-vis-NIR spectra of ICGJ@PEI PEG-PLGA polymersomes after purification at various pH. The optimization was carried out at 100 μL of added PEI. FIG. 10B depicts UV-vis-NIR spectra of ICGJ@PEI PEG-PLGA polymersomes after purification at various PEI volumes for preparation of ICGJ@PEI NPs at pH 4. The optimization was carried out at pH 4. FIG. 10C depicts a typical TEM (blue) and DLS) (yellow) volume distributions of ICGJ@PEI Ps (The average and std. dev. are given in Table 8 for DO. FIG. 10D depicts representative TEM images of ICGJ@PEI PEG-PLGA polymersomes with additional examples in FIG. 16. Most Ps only contained only a single ICGJ, and a small population included two ICGJ. The inset figure in the bottom-right panel shows an empty polymersome with a similar size. Scale bar indicates 100 nm.

FIG. 11 depicts exemplary results that demonstrate encapsulation of ICGJ@PEI1.8k¬NPs (prepared at pH 4) with varying concentrations and no ICGJ in W2 phase. As seen above, encapsulation of 50 mg/mL ICGJ@PEI NPs without the presence of 5 mg/mL ICGJ in the W₂ phase significantly reduces the intensity of the J aggregate peak. This reduction is mitigated by increasing the concentration of ICGJ@PEI in the W₁ phase to 150 mg/mL.

FIG. 12 depicts exemplary results that demonstrate encapsulation of ICGJ@PEI1.8k¬NPs (prepared at pH 4) with varying concentrations and 5 mg/mL ICGJ in W₂ phase. The increase in the concentration of ICGJ@PEI in the W₁ phase resulted in a higher J aggregate peak intensity.

FIG. 13 depicts exemplary results that demonstrate encapsulation of ICGJ@PEI1.8k¬NPs (prepared at pH 4) with 5 mg/mL ICG, ICGJ, and ICGJ@PEI in W₂ phase. ICGJ in the W₂ phase provides the most effective sacrificial layer as individual ICG molecules leave ICGJ NPs to cover the interface. Given that the solubility limit of ICG in aqueous solutions is 1.15 mg/mL and the dissociation rate is slower for ICGJ@PEI, the results shown here support the proposed mechanism as discussed in Example 1.

FIG. 14, comprising FIG. 14A and FIG. 14E, depicts exemplary characterization and stability results of polymersomes. FIG. 14A depicts key properties of ICGJ@PEI and ICGJ@PEI Ps as further described in Table 1. FIG. 14B depicts hydrodynamic diameters of empty Ps and ICGJ@PEI Ps in DI water. FIG. 14C depicts hydrodynamic diameters of empty Ps and ICGJ@PEI Ps in 100% FBS. FIG. 14D depicts normalized UV-vis-NIR spectra to unity at 780 nm of ICGJ@PEI polymersomes versus time in DI H₂O at pH 5.5 to emphasize relative changes in the 890 nm peak. To set pH at 5.5, HCl was added. FIG. 14E depicts normalized UV-vis-NIR spectra to unity at 780 nm of ICGJ@PEI polymersomes versus time in FBS at pH 5.5 to emphasize relative changes in the 890 nm peak. To set pH at 5.5, HCl was added.

FIG. 15 depicts histogram representations of PEG-PLGA polymersomes sizes, measured by TEM (blue) and DLS (yellow) at different ultrasonication intensity. The reported TEM size distribution is weight-normalized and multiple grids/samples have been used for the sizing. The DLS size distribution is volume-weighted and the shown histogram is a representative of at least 3 measurements.

FIG. 16 depicts exemplary additional TEM images of polymersomes with a mean diameter of (top) 77 nm, (middle) 92 nm, and (bottom) 106 nm. The averages are based on different sonication intensities (30%, 25% and 20% respectively); a detailed distribution for each sample is shown in FIG. 15. For 77 nm polymersomes (the optimized system), a shell thickness on the order of 10 to 15 nm is used as input in the calculation of the percentage of filled capsules.

FIG. 17, comprising FIG. 17A and FIG. 17B, depicts exemplary PEG-PLGA empty polymersomes sizes and the structure of PEG₁₀ₖ-PLGA₉₀ₖ. FIG. 17A depicts histogram representations of exemplary PEG-PLGA empty polymersomes sizes, measured by DLS. The weighted average of the three measurements is 76±3 nm. FIG. 17B depicts structure of PEG₁₀ₖ-PLGA₉₀ₖ, forming the shell of the Polymersomes.

FIG. 18 depicts representative ¹H NMR spectrum for 10 mg/mL PEG₁₀ₖ.

FIG. 19 depicts exemplary results that demonstrate evolution of the size of ICGJ@PEI polymersomes. The mechanistic details of this evolution are described in Example 1. Initially, the majority of the emulsion had a D_{H} of 460 nm. The shrinkage of the oil droplets may be observed over 6 hours. During the first 3 hours a second population with smaller particles at nm becomes predominant, which represents the polymersomes, as shown by the black line in FIG. 3D. Furthermore, the volume fraction associated with the oil droplets decreases.

FIG. 20 depicts exemplary results that demonstrate effect of initial PEG-PLGA concentration in chloroform phase on ICG encapsulation efficiency and polymer recovery rate. A value of 25 mg/mL PEG-PLGA was used throughout this study based on this result.

FIG. 21, comprising FIG. 21A and FIG. 21B, depicts exemplary results that demonstrate viability of cancer cell lines relative to untreated controls measured by MTS assay (mean±STD, N=4). The cells were exposed to ICGJ@PEI Ps at 6.25 μg/mL for 24 hours. No statistically significant differences were detected by the Student's t-test. FIG. 21A depicts exemplary results that demonstrate viability of breast cancer cell lines relative to untreated controls measured by MTS assay (mean±STD, N=4). The cells were exposed to ICGJ@PEI Ps at 6.25 μg/mL for 24 hours. FIG. 21B depicts exemplary results that demonstrate viability of ovarian cancer cell lines relative to untreated controls measured by MTS assay (mean±STD, N=4). The cells were exposed to ICGJ@PEI Ps at 6.25 μg/mL for 24 hours.

FIG. 22, comprising FIG. 22A through FIG. 22D, depicts exemplary PAI of breast and ovarian cancer cells. Labels+, *, and #indicate statistically significant differences in paired comparisons of PA signals from labeled cells at 890 nm. A one-way ANOVA was used to determine statistical significance (p<0.05). PA data shown with the same dynamic range across comparisons. FIG. 22A depicts cross-sectional PA images at 890 nm excitation overlaid B-mode ultrasound of gelatin phantoms containing inclusions of breast cancer cells MDA-MB-468, MDA-MB-231, and MDA-MB-435 labeled with either EGFR-targeted (ICGJ@PEI Ps-Ab) or non-targeted (ICGJ@PEI Ps) polymersomes. FIG. 22B depicts exemplary corresponding PA spectra that are based on volumetric PA signal analyses of breast cancer cells (each value shows mean±STD based on n=3 samples). FIG. 22C depicts cross-sectional PA images at 890 nm excitation overlaid B-mode ultrasound of gelatin phantoms containing inclusions of ovarian cancer cells SKOV3 and A2780 labeled with either EGFR-targeted (ICGJ@PEI Ps-Ab) or non-targeted (ICGJ@PEI Ps) polymersomes. FIG. 22D depicts exemplary corresponding PA spectra that are based on volumetric PA signal analyses of ovarian cancer cells (each value shows mean±STD based on n=3 samples).

FIG. 23, comprising FIG. 23A and FIG. 23B, depicts exemplary PA spectra of SKOV3 and A2780 cells. FIG. 23A depicts exemplary PA spectra of SKOV3 cells labeled with either EGFR-targeted (ICGJ@PEI Ps-Ab) or non-targeted (ICGJ@PEI Ps) polymersomes for 2 and 6 h. FIG. 23B depicts exemplary PA spectra of A2780 cells labeled with either EGFR-targeted (ICGJ@PEI Ps-Ab) or non-targeted (ICGJ@PEI Ps) polymersomes for 2 and 6 h.

FIG. 24, comprising FIG. 24A and FIG. 24B, depicts exemplary PA spectra of SKOB3 and MDA-MB-468 cells. FIG. 24A depicts exemplary PA spectra of SKOV3 cells labeled with ICGJ@PEI Ps-Ab acquired from the same region over three continuous scans. FIG. 24B depicts exemplary PA spectra of MDA-MB-468 cells labeled with ICGJ@PEI Ps-Ab acquired from the same region over three continuous scans.

FIG. 25, comprising FIG. 25A through FIG. 25F, depicts exemplary results that demonstrate PA detection sensitivity of labeled cancer cells. Error bars are based on three independent samples (mean±STD, n=3). PA data shown with the same dynamic range across comparisons. FIG. 25A depicts PA signal intensity at 870, 880, and 895 nm as a function of cell concentration for MDA-MB-468 cells labeled with EGFR-targeted polymersomes (ICGJ@PEI Ps-Ab). FIG. 25B depicts linear regression fits of PA signals at 870 nm as a function of serial dilutions of MDA-MB-468 cells labeled with ICGJ@PEI Ps-Ab. FIG. 25C depicts corresponding cross-sectional PA images at 870 nm overlaid B-mode ultrasound of labeled MDA-MB-468 at various dilutions. FIG. 25D depicts PA signal intensity at 870, 880, and 895 nm as a function of cell concentration for SKOV3 cells labeled with EGFR-targeted polymersomes (ICGJ@PEI Ps-Ab). FIG. 25E depicts linear regression fits of PA signals at 870 nm as a function of serial dilutions of SKOV3 cells labeled with ICGJ@PEI Ps-Ab. FIG. 25F depicts corresponding cross-sectional PA images at 870 nm overlaid B-mode ultrasound of labeled SKOV3 at various dilutions.

FIG. 26 depicts exemplary calibration curve of ICG absorbance at 790 nm wavelength vs. ICG concentration. The ICG was dispersed in DI water only.

FIG. 27 depicts exemplary calibration curve of ICG absorbance at 790 nm wavelength vs. ICG concentration. The ICG was dissolved in 1/1 v/v ethanol/DI water solution.

FIG. 28 depicts exemplary calibration curve of ICG absorbance at 790 nm wavelength vs. ICG concentration. The ICG was dispersed in DI water only.

FIG. 29 depicts exemplary standard calibration curve of Alexa Fluor 488-labeled antibodies.

FIG. 30 depicts exemplary additional TEM images (left) and high resolution TEM images (right) of ICGJ NPs. The TEM weight average of the ICGJ NPs is 47±4 nm. At least 50 particles were sized for the histogram shown in FIG. 1C.

FIG. 31 depicts exemplary additional TEM images of ICGJ@PEI1.8k NPs. The TEM volume average diameter of the ICGJ@PEI— NPs is 53±4 nm. At least 50 particles were sized for the histogram shown in FIG. 1D (left). Additional high resolution TEM images of ICGJ@PEI NPs. The contrast was not sufficient given similar electron densities to discern the thin PEI shell (right).

FIG. 32 depicts TEM images of empty Ps. The scale bar is 50 nm. A shell thickness on the order of 7 to 15 nm is used as input in the calculation of the percentage of filled capsules.

FIG. 33 depicts exemplary evolution of UV-vis-NIR spectra for ICGJ@PEI loaded polymersomes after each round of purification (top); Normalized ICGJ/ICG absorbance ratio after each purification step of ICGJ@PEI loaded polymersomes (bottom). During the three rounds of centrifugal filtration to remove unencapsulated ICGJ that is dissociated with the 10% ethanol solution, a modest decrease in the I₈₉₅/I₇₉₀ ICGJ polymersomes spectra for the product in the retentate is observed.

FIG. 34 depicts representative calibration curve of PEG₁₀ₖ on the basis of the peak area to the area of the TMS reference peak.

FIG. 35 depicts representative ¹H NMR spectrum for PLGA dissolved in CDCl₃ (top), empty PEG₁₀ₖ-PLGA₉₀ₖ polymersomes dispersed in D₂O (middle), and empty PEG₁₀ₖ-PLGA₉₀ₖ Polymersomes dissolved in CDCl₃ (bottom).

FIG. 36 depicts percentage of capsules containing an ICGJ@PEI aggregate with increasing assumed average shell thickness of the Polymersomes. With increasing shell thickness, the number of Polymersomes become smaller. Therefore, the percentage of capsules that are occupied with ICGJ@PEI NPs increases. The curve is an average of three separate experiments. The typical uncertainty in the % full capsules is ˜0.1.

FIG. 37 depicts exemplary results demonstrating dissociation kinetics of Polymersomes in RPMI at 37° C. at pH 5.5 and 7.4 over the course of three weeks.

FIG. 38 depicts UV-vis-NIR spectra of ICGJ@PEI Polymersomes in (top) DI water, (middle) RPMI, and (bottom) FBS at pH 7.4 in 37° C. Here the changes are much slower than at pH 5.5.

FIG. 39 depicts fluence of LZ250 transducer measured on the surface (blue) and at 11 mm depth of a gelatin phantom (red). This experiment shows no significant influence of depth on spectral fluence distribution in the phantom geometry that was used in our photoacoustic experiments.

FIG. 40 depicts exemplary UV-vis-NIR spectra of six separate ICGJ NP samples.

FIG. 41 depicts exemplary three additional sets of stability and dissociation for ICGJ NPs in DI water and FBS in pH 5.5 and 7.4. The average diameters and error bars reported in FIG. 7 are based on these data points.

FIG. 42 depicts exemplary UV-vis-NIR spectra of six separate ICGJ@PEI NP samples.

FIG. 43 depicts exemplary three additional sets of stability and dissociation for ICGJ@PEI NPs in DI water and FBS in pH 5.5 and 7.4. The average diameters and error bars reported in FIG. 7 are based on these data points.

FIG. 44 depicts exemplary UV-vis-NIR spectra of nine separate ICGJ@PEI P samples based on optimized conditions. The ratio of ICGJ to ICG peak is 1.6±0.1.

FIG. 45 depicts exemplary dissociation kinetics of empty and ICGJ@PEI loaded Polymersomes in DI water in pH values of 5.5 and 7.4 at 37° C.

FIG. 46 depicts exemplary dissociation kinetics of empty and ICGJ@PEI loaded Polymersomes in 100% FBS in pH values of 5.5 and 7.4 at 37° C.

FIG. 47 depicts exemplary release kinetics of ICG from Polymersomes during the dissociation process at DI and 100% FBS at pH 5.5 and 7.4. The bottom figures are reproduced data.

FIG. 48 depicts exemplary UV-vis-NIR spectrum of the final polymersomes after purification. The I₈₉₅/I₇₉₀ ratio is 1.6.

FIG. 49 depicts exemplary UV-vis-NIR spectrum of the final polymersomes after purification. The I₈₉₅/I₇₉₀ ratio is 2.4.

FIG. 50, comprising FIG. 50A and FIG. 50B, depicts exemplary UV-vis-NIR and the volume-averaged size distribution spectra of the as-purified polymersomes without PEI. FIG. 50A depicts an exemplary UV-vis-NIR spectrum of as-purified polymersomes without PEI. FIG. 50B depicts an exemplary volume-averaged size distribution of polymersomes obtained by DLS.

FIG. 51, comprising FIG. 51A and FIG. 51B, depicts exemplary TEM images of the polymersomes obtained using the AMT Imaging System (Advanced Microscopy Techniques Corp., Danvers, MA) at an accelerating voltage of 80 Kv. FIG. 51A depicts an exemplary TEM image of the polymersomes obtained using the AMT Imaging System (Advanced Microscopy Techniques Corp., Danvers, MA) at an accelerating voltage of 80 Kv. FIG. 51B depicts an exemplary TEM image of the polymersomes obtained using the AMT Imaging System (Advanced Microscopy Techniques Corp., Danvers, MA) at an accelerating voltage of 80 Kv.

FIG. 52, comprising FIG. 52A and FIG. 52B, depicts exemplary changes in spectra of polymersomes without PEI in 10% ethanol at different times. FIG. 52A depicts the change in the UV-vis-NIR spectra of polymersomes without PEI in 10% ethanol at different times. FIG. 52B depicts the change in the ratio of ICGJ to ICG monomer in 10% ethanol solution.

FIG. 53, comprising FIG. 53A and FIG. 53B, depicts exemplary changes in spectra of polymersomes without PEI in 100% FBS at pH=7.4. FIG. 53A depicts the change in the UV-vis-NIR spectra of polymersomes without PEI in 100% FBS at pH=7.4 FIG. 53B depicts the change in the ratio of ICGJ to ICG monomer in 100% FBS solution at pH=7.4.

FIG. 54, comprising FIG. 54A and FIG. 54B, depicts exemplary results of the new centrifuge purification method for ICGJ@PEI Ps. FIG. 54A depicts the bottom of the filter tube that shows that during centrifugation with CF some ICG/partially destroyed Ps deposits. FIG. 54B depicts exemplary UV-vis-NIR spectra of the supernatant, deposit, and product obtained using standard method.

FIG. 55 depicts exemplary results demonstrating stability of Ps supernatant-higher ratio I₈₉₅/I₇₈₀ of 2.4 in pure FBS (At day 5 large precipitation-visible protein aggregation).

FIG. 56 depicts exemplary results and exemplary UV-vis-NIR spectra of samples obtained using ultracentrifugation purification method (5200 rpm for 15 minutes).

FIG. 57 depicts exemplary results that demonstrate loss of sample after ultracentrifugation.

FIG. 58 depicts exemplary deposit of PEG-PLGA.

FIG. 59 depicts exemplary results obtained after washing of ICGJ@PEI Ps using CF 30K or 100K.

FIG. 60 depicts exemplary UV-vis-NIR spectra of ICGJ@PEI Ps washed using CF 30K (left) and 100K (right).

FIG. 61 depicts exemplary UV-vis-NIR spectra of ICGJ@PEI Ps was 10 times using CF 30K (left) and 100K (right).

FIG. 62, comprising FIG. 62A and FIG. 62B, depicts representative UV-Vis spectra and volume-averaged size distribution of Ps without PEI. FIG. 62A depicts representative UV-Vis spectra of as-purified and heated Ps without PEI. FIG. 62B depicts representative volume-averaged size distribution of Ps without PEI obtained by DLS using NNLS model.

FIG. 63, comprising FIG. 63A and FIG. 63B, depicts representative change in the UV-Vis spectra of Ps without PEI and in the I₈₉₀/I₇₈₅ ratio of ICGJ to ICG monomer in 80% FBS final concentration and 0.04 mg/mL ICG. FIG. 63A depicts representative change in the UV-Vis spectra of Ps without PEI in ˜80% FBS final concentration (i.e., added around 0.8 mL of 100% FBS) and 0.04 mg/mL ICG final concentration (i.e., added 0.2 mL of Ps at 0.2 mg/mL ICG) at pH=7.4 with a total volume of solution of 1 mL. FIG. 63B depicts representative the change in the I₈₉₀/I₇₈₅ ratio of ICGJ to ICG monomer in 80% FBS solution and 0.04 mg/mL ICG at pH=7.4.

FIG. 64, comprising FIG. 64A and FIG. 64B, depicts representative UV-Vis spectra and volume averaged size distribution of Ps samples without PEI stored in excess polyvinyl alcohol (PVA). FIG. 64A depicts representative UV-Vis spectra of as-purified Ps samples without PEI stored in excess PVA. FIG. 64B depicts representative volume-averaged size distribution of Ps without PEI stored in excess PVA obtained by DLS using NNLS model.

FIG. 65, comprising FIG. 65A and FIG. 65B, depicts representative change in the UV-Vis spectra of Ps without PEI at 0.05 mg/mL ICG in 10% ethanol and in the I₈₉₀/I₇₈₅ ratio of ICGJ to ICG monomer in 10% ethanol solution. FIG. 65A depicts representative change in the UV-Vis spectra of Ps without PEI at 0.05 mg/mL ICG in 10% ethanol and 1 mL total volume at different times. FIG. 65B depicts representative change in the I₈₉₀/I₇₈₅ ratio of ICGJ to ICG monomer in 10% ethanol solution.

FIG. 66, comprising FIG. 66A and FIG. 66B, depicts representative change in the UV-Vis spectra of Ps without PEI and in the I₈₉₀/I₇₈₅ ratio of ICGJ to ICG monomer in ˜80% FBS final concentration and 0.04 mg/mL ICG. FIG. 66A depicts representative change in the UV-Vis spectra of Ps without PEI in ˜80% FBS final concentration (i.e., added around 0.8 mL of 100% FBS) and 0.04 mg/mL ICG final concentration (i.e., added 0.2 mL of Ps at 0.2 mg/mL ICG) at pH=7.4 with a total volume of solution of 1 mL. FIG. 66B depicts representative change in the I₈₉₀/I₇₈₅ ratio of ICGJ to ICG monomer in 80% FBS solution and mg/mL ICG at pH=7.4.

FIG. 67 depicts representative optical density of PS containing ICGJ. PEI was not present in these Ps. The three experiments demonstrate the reproducibility in the experiment.

### DETAILED DESCRIPTION

The present invention is based, in part, on the discovery of stable nanoparticles comprising indocyanine green J-aggregate (ICGJ). In various aspects of the invention, the nanoparticles optionally comprise a cationic polymer. In one embodiment, the cationic polymer is PEI. In some aspects, the nanoparticle further comprises a poly(lactide-co-glycolic acid)-polyethylene glycol (PLGA-PEG). In some embodiments, the nanoparticle forms polymersome by encapsulating the ICGJ and PEI in the PLGA-PEG. Thus, in some aspects, the invention provides compositions comprising ICGJ encapsulated in a nanoparticle or polymersome.

In various aspects of the invention, the nanoparticles are cationic polymer-free nanoparticles. In some embodiments, the nanoparticles are polyethyleneimine (PEI)-free nanoparticles.

In various aspects of the invention, the nanoparticles have significant absorption peaks at 680 and 1100 nm wavelength. In some embodiments, the nanoparticles comprise a targeting domain (e.g., a monoclonal antibody) that directs the nanoparticle to a specific cell or tissue (e.g. cancer cell). Thus, the present invention relates to compositions and methods relating to nanoparticles that selectively target cells associated with diseases or disorders (e.g., cancer cells) and can be used for detection, imaging, and treatment of diseases or disorders in a subject. The present invention additionally provides kits that find use in the practice of the methods of the invention.

### Definitions

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, the preferred methods and materials are described.

As used herein, each of the following terms has the meaning associated with it in this section.

The articles “a” and “an” are used herein to refer to one or to more than one (i.e., to at least one) of the grammatical object of the article. By way of example, “an element” means one element or more than one element.

The term “about” will be understood by persons of ordinary skill in the art and will vary to some extent depending on the context in which it is used. As used herein when referring to a measurable value such as an amount, a temporal duration, and the like, the term “about” is meant to encompass variations of ±20% or ±10%, more preferably ±5%, even more preferably ±1%, and still more preferably ±0.1% from the specified value, as such variations are appropriate to perform the disclosed methods.

The terms “patient,” “subject,” “individual,” and the like are used interchangeably herein, and refer to any animal, or cells thereof whether in vitro or in situ, amenable to the methods described herein. In certain non-limiting embodiments, the patient, subject or individual is a human.

The term “biological tissue” as used herein refers to a collection of interconnected cells and extracellular matrix that perform a similar function or functions within an organism. Examples of biological tissues include, but are not limited to, connective tissue, muscle tissue, nervous tissue (of the brain, spinal cord, and nerves), epithelial tissue, organ tissue, cancer tissue, and any combination thereof. Connective tissue includes fibrous tissue like fascia, tendon, ligaments, heart valves, bone, and cartilage. Muscle tissue includes skeletal muscle tissue, smooth muscle tissue, such as esophageal, stomach, intestinal, bronchial, uterine, urethral, bladder, and blood vessel tissue, and cardiac muscle tissue. Epithelial tissue includes simple epithelial tissue, such as alveolar epithelial tissue, blood vessel endothelial tissue, and heart mesothelial tissue, and stratified epithelial tissue.

The term “abnormal” when used in the context of organisms, tissues, cells or components thereof, refers to those organisms, tissues, cells or components thereof that differ in at least one observable or detectable characteristic (e.g., age, treatment, time of day, etc.) from those organisms, tissues, cells or components thereof that display the “normal” (expected) respective characteristic. Characteristics which are normal or expected for one cell or tissue type, might be abnormal for a different cell or tissue type.

A “disease” is a state of health of an animal wherein the animal cannot maintain homeostasis, and wherein if the disease is not ameliorated then the animal's health continues to deteriorate.

In contrast, a “disorder” in an animal is a state of health in which the animal is able to maintain homeostasis, but in which the animal's state of health is less favorable than it would be in the absence of the disorder. Left untreated, a disorder does not necessarily cause a further decrease in the animal's state of health.

“Cancer,” as used herein, refers to the abnormal growth or division of cells. Generally, the growth and/or life span of a cancer cell exceeds, and is not coordinated with, that of the normal cells and tissues around it. Cancers may be benign, pre-malignant or malignant. Cancer occurs in a variety of cells and tissues, including, but not limited to, the oral cavity (e.g., mouth, tongue, pharynx, etc.), digestive system (e.g., esophagus, stomach, small intestine, colon, rectum, liver, bile duct, gall bladder, pancreas, etc.), respiratory system (e.g., larynx, lung, bronchus, etc.), bones, joints, skin (e.g., basal cell, squamous cell, meningioma, etc.), breast, genital system, (e.g., uterus, ovary, prostate, testis, etc.), urinary system (e.g., bladder, kidney, ureter, etc.), eye, nervous system (e.g., brain, etc.), endocrine system (e.g., thyroid, etc.), soft tissues (e.g., muscle, fat, etc.), and hematopoietic system (e.g., lymphoma, myeloma, leukemia, acute lymphocytic leukemia, chronic lymphocytic leukemia, acute myeloid leukemia, chronic myeloid leukemia, etc.).

As used herein, the term “diagnosis” refers to the determination of the presence of a disease or disorder. In some embodiments of the present invention, methods for making a diagnosis are provided which permit determination of the presence of a particular disease or disorder.

A “therapeutic” treatment is a treatment administered to a subject who exhibits signs or symptoms of a disease or disorder, for the purpose of diminishing or eliminating those signs or symptoms.

As used herein, “treating a disease or disorder” means reducing the severity and/or frequency with which a sign or symptom of the disease or disorder is experienced by a subject.

A disease or disorder is “alleviated” if the severity of a sign or symptom of the disease or disorder, the frequency with which such a sign or symptom is experienced by a subject, or both, is reduced.

The term “derivative” refers to a small molecule that differs in structure from the reference molecule, but retains the essential properties of the reference molecule. A derivative may change its interaction with certain other molecules relative to the reference molecule. A derivative molecule may also include a salt, an adduct, tautomer, isomer, or other variant of the reference molecule.

The term “tautomers” are constitutional isomers of organic compounds that readily interconvert by a chemical process (tautomerization).

The term “isomers” or “stereoisomers” refer to compounds, which have identical chemical constitution, but differ with regard to the arrangement of the atoms or groups in space.

The term “H-aggregate” as used herein refers to aggregates of monomers of a compound (e.g., a dye) whereupon aggregation causes a shift in the extinction to the lower wavelengths.

The term “J-aggregate” as used herein refers to aggregates of monomers of a compound (e.g., a dye) whereupon aggregation causes a shift in the extinction to higher wavelengths.

As used herein, the term “nanoparticle” refers to particles having a particle size on the nanometer scale, less than 1 micrometer. For example, the nanoparticle may have a particle size up to about 50 nm. In another example, the nanoparticle may have a particle size up to about nm. In another example, the nanoparticle may have a particle size up to about 6 nm. As used herein, “nanoparticle” refers to a number of nanoparticles, including, but not limited to, nanoclusters, nanocapsules, core-shell nanocapsules, nanovesicles, micelles, block copolymer micelles, lamaellae shaped particles, polymersomes, dendrimers, and other nano-size particles of various other small fabrications that are known to those of skill in the art. The shapes and compositions of nanoparticles may be guided during condensation of atoms by selectively favoring growth of particular crystal facets to produce spheres, rods, wires, discs, cages, core-shell structures and many other shapes. The definitions and understandings of the entities falling within the scope of nanocapsule are known to those of skill in the art, and such definitions are incorporated herein by reference and for the purposes of understanding the general nature of the subject matter of the present application. However, the following discussion is useful as a further understanding of some of these terms.

For example, the term “nanocapsule” refers to a vesicular system or hollow particle with a shell surrounding a core-forming space, which, in certain instances, can be used for transporting a payload on a nanoscale level. A nanocapsule may also be a nano-sized version of a container. The payload of the nanocapsule can be, but is not limited to drugs, medicaments, pharmaceutical compositions, chemical compositions, therapeutic compositions, biological macromolecules, dyes, biological material, immunological compositions, nutritional compositions, vitamins, proteins, nucleic acids, antibodies and vaccines. Various materials may be used for producing such nanocapsules. Nanocapsule refers to a particle having a hollow core that is surrounded by a shell, such that the particle has a size of less than about 1000 nanometers. When a nanocapsule includes a bioactive component, the bioactive component is located in the core that is surrounded by the shell of the nanocapsule.

As used herein, the term “nanocage” refers to a nanocapsule, whereby the shell is not solid, as described for the nanocapsule, but has multiple holes or pores in its shell, thereby making it possible for the payload within the core of the nanocage to come into contact with the surrounding environment. These holes or pores may be regular or irregular in shape and/or spacing on the surface of the particle.

The term “micelle”, a useful article in the employment of a general aspect of the present invention, can generally be thought of as a small—on the order of usually nanometers in diameter—aggregate of amphiphilic linear molecules having a polar, or hydrophilic end and an opposite non-polar, or hydrophobic end. These linear molecules can be comprised of simple molecules, or polymeric chains. A micelle can also be referred to as an aggregate of surfactant molecules dispersed in a liquid colloid. A typical micelle in aqueous solution can form an aggregate with the hydrophilic “head” regions in contact with surrounding solvent, and the sequestering of the hydrophobic tail regions in the micelle center. Other and similar definitions, descriptions and understandings of micelles are also known to those of skill in the art and are incorporated herein by reference.

The term “polymersome” as used herein refers to a vesicle-type which is typically composed of block copolymer amphiphiles, i.e., synthetic amphiphiles that have an amphiphilicity similar to that of lipids. By virtue of their amphiphilic nature (having a more hydrophilic block (head) and a more hydrophobic block (tail)), the block copolymers are capable of self-assembling into a head-to-tail and tail-to-head bilayer structure similar to liposomes. Compared to liposomes, polymersomes have much larger molecular weights, with number average molecular weights typically ranging from 1000 to 100000, preferably of from 2500 to 50000 and more preferably from 5000 to 25000, are typically chemically more stable, less leaky, less prone to interfere with biological membranes, and less dynamic due to a lower critical aggregation concentration. These properties result in less opsonisation and longer circulation times. The terms “more hydrophilic” and “more hydrophobic” as used in the context of the ampohiphilic nature of the block copolymers are used in a relative sense. i.e., both can be either hydrophilic or hydrophobic, as long as the difference in polarity between the blocks is sufficient for the formation of polymersomes according to the present invention. In view of the creation of a cavity in which water may be incorporated, it is preferred for the more hydrophilic end of the polymer to be hydrophilic per se. Further, in view of the use as a therapeutic agent carrier, it is desired that also hydrophobic and/or hydrophilic therapeutic agents can be incorporated into the polymersomes. In one embodiment, the hydrophobic end of the polymer is hydrophobic per se. The amphiphilic nature of the block copolymers is preferably realized in the form of a block copolymer comprising a block made up of more hydrophilic monomeric units (A) and a block made up of more hydrophobic units (B), the block copolymer having the general structure AₙBₘ, with n and m being integers of from 5 to 5000, 10 to 1000, or 10 to 500. It is also conceivable that one or more further units or blocks are built-in, e.g., a unit C with an intermediate hydrophilicity so as to yield a terpolymer having the general structure AₙCₚBₘ, with n and m being as defined above, and p being an integer of from 5 to 5000, preferably 10 to 1000, more preferably 10 to 500. Any of the blocks can itself be a copolymer, i.e., comprise different monomeric units of the required hydrophilic respectively hydrophobic nature. In one embodiment, the blocks themselves are homopolymeric. Any of the blocks, in particular the more hydrophilic block, may bear charges. The number and type of charges may depend on the pH of the environment. Any combination of positive and/or negative charges on any of the blocks is contemplated by the present invention.

“Dendrimers” have descriptions, definitions and understandings in the literature. For example, and without limitation and including other and similar definitions, descriptions and understandings in the art, the term dendrimer from the Greek word, “dendron”, for tree, can refer to a synthetic, three-dimensional molecule with branching parts. Descriptions and understandings of dendrimers can be gleaned from Holister et al., Dendrimers, Technology White Papers nr. 6, pub. October 2003 by cientifica, as well as the other literature published by those skilled in the art on dendrimers, all of which are incorporated herein by reference.

“Lamella” is a term whose definitions, descriptions and understandings are also known to those of skill in the art and which are incorporated herein by reference. In a very general sense, lamella or lamellae refers to plate-like, gill-shaped or other layered structures.

The definitions, descriptions and understandings of “nanovesicle” are well known to those of skill in the art, and are incorporated herein by reference. For example, “nanovesicle” can refer to a variety of small sac, sac-like or globular structures capable of containing fluid or other material therein

As used herein, the term “polymorph” refers to crystalline forms having the same chemical composition but different spatial arrangements of the molecules, atoms, and/or ions forming the crystal.

“Pharmaceutically acceptable” refers to those properties and/or substances which are acceptable to the subject from a pharmacological/toxicological point of view and to the manufacturing pharmaceutical chemist from a physical/chemical point of view regarding composition, formulation, stability, subject acceptance and bioavailability. “Pharmaceutically acceptable carrier” refers to a medium that does not interfere with the effectiveness of the biological activity of the active ingredient(s) and is not toxic to the host to which it is administered.

As used herein, the term “pharmaceutically acceptable carrier” means a pharmaceutically acceptable material, composition or carrier, such as a liquid or solid filler, stabilizer, dispersing agent, suspending agent, diluent, excipient, thickening agent, solvent or encapsulating material, involved in carrying or transporting a compound useful within the invention within or to the subject such that it may perform its intended function. Typically, such constructs are carried or transported from one organ, or portion of the body, to another organ, or portion of the body. Each carrier must be “acceptable” in the sense of being compatible with the other ingredients of the formulation, including the compound useful within the invention, and not injurious to the subject. Some examples of materials that may serve as pharmaceutically acceptable carriers include: sugars, such as lactose, glucose, and sucrose; starches, such as corn starch and potato starch; cellulose, and its derivatives, such as sodium carboxymethyl cellulose, ethyl cellulose, and cellulose acetate; powdered tragacanth; malt; gelatin; talc; excipients, such as cocoa butter and suppository waxes; oils, such as peanut oil, cottonseed oil, safflower oil, sesame oil, olive oil, corn oil, and soybean oil; glycols, such as propylene glycol; polyols, such as glycerin, sorbitol, mannitol, and polyethylene glycol; esters, such as ethyl oleate and ethyl laurate; agar; buffering agents, such as magnesium hydroxide and aluminum hydroxide; surface active agents; alginic acid; pyrogen-free water; isotonic saline; Ringer's solution; ethyl alcohol; phosphate buffer solutions; and other non-toxic compatible substances employed in pharmaceutical formulations. As used herein, “pharmaceutically acceptable carrier” also includes any and all coatings, antibacterial and antifungal agents, and absorption delaying agents, and the like that are compatible with the activity of the compound useful within the invention, and are physiologically acceptable to the subject. Supplementary active compounds may also be incorporated into the compositions. The “pharmaceutically acceptable carrier” may further include a pharmaceutically acceptable salt of the compound useful within the invention. Other additional ingredients that may be included in the pharmaceutical compositions used in the practice of the invention are known in the art.

The term “pharmaceutically acceptable salt” refers to any pharmaceutically acceptable salt, which upon administration to the subject is capable of providing (directly or indirectly) a compound as described herein. Such salts preferably are acid addition salts with physiologically acceptable organic or inorganic acids. Examples of the acid addition salts include mineral acid addition salts such as, for example, hydrochloride, hydrobromide, hydroiodide, sulphate, nitrate, phosphate, and organic acid addition salts such as, for example, acetate, trifluoroacetate, maleate, fumarate, citrate, oxalate, succinate, tartrate, malate, mandelate, methane sulphonate, and p-toluenesulphonate. Examples of the alkali addition salts include inorganic salts such as, for example, sodium, potassium, calcium and ammonium salts, and organic alkali salts such as, for example, ethylenediamine, ethanolamine, N,N-dialkylenethanolamine, triethanolamine, and basic amino acids salts. However, it will be appreciated that non-pharmaceutically acceptable salts also fall within the scope of the invention since those may be useful in the preparation of pharmaceutically acceptable salts. Procedures for salt formation are conventional in the art.

The term “solvate” in accordance with this invention should be understood as meaning any form of the active compound in accordance with the invention in which the said compound is bonded by a non-covalent bond to another molecule (normally a polar solvent), including especially hydrates and alcoholates.

As used herein, the term “pharmaceutical composition” refers to a mixture of at least one compound of the invention with other chemical components and entities, such as carriers, stabilizers, diluents, dispersing agents, suspending agents, thickening agents, and/or excipients. The pharmaceutical composition facilitates administration of the compound to an organism. Multiple techniques of administering a compound exist in the art including, but not limited to, intravenous, oral, aerosol, parenteral, ophthalmic, pulmonary and topical administration.

As used herein, the terms “therapeutic compound”, “therapeutic agent”, “drug”, “active pharmaceutical”, and “active pharmaceutical ingredient” are used interchangeably to refer to chemical entities that display certain pharmacological effects in a body and are administered for such purpose. Non-limiting examples of therapeutic agents include, but are not limited to, hydrophilic therapeutic agents, hydrophobic therapeutic agents, antibiotics, antibodies, small molecules, anti-cancer agents, chemotherapeutic agents, immunomodulatory agents, RNA molecules, siRNA molecules, DNA molecules, gene editing agents, gene-silencing agents, CRISPR-associated agents (e.g., guide RNA molecules, endonucleases, and variants thereof), analgesics, vaccines, anticonvulsants; anti-diabetic agents, antifungal agents, antineoplastic agents, anti-parkinsonian agents, anti-rheumatic agents, appetite suppressants, biological response modifiers, cardiovascular agents, central nervous system stimulants, contraceptive agents, dietary supplements, vitamins, minerals, lipids, saccharides, metals, amino acids (and precursors), nucleic acids and precursors, contrast agents, diagnostic agents, dopamine receptor agonists, erectile dysfunction agents, fertility agents, gastrointestinal agents, hormones, immunomodulators, antihypercalcemia agents, mast cell stabilizers, muscle relaxants, nutritional agents, ophthalmic agents, osteoporosis agents, psychotherapeutic agents, parasympathomimetic agents, parasympatholytic agents, respiratory agents, sedative hypnotic agents, skin and mucous membrane agents, smoking cessation agents, steroids, sympatholytic agents, urinary tract agents, uterine relaxants, vaginal agents, vasodilator, anti-hypertensive, hyperthyroids, anti-hyperthyroids, anti-asthmatics and vertigo agents. In certain embodiments, the one or more therapeutic agents are water-soluble, poorly water-soluble drug or a drug with a low, medium or high melting point. The therapeutic agents may be provided with or without a stabilizing salt or salts.

Some examples of active ingredients suitable for use in the pharmaceutical formulations and methods of the present invention include: hydrophilic, lipophilic, amphiphilic or hydrophobic, and that can be solubilized, dispersed, or partially solubilized and dispersed, on or about the nanocluster. The active agent-nanocluster combination may be coated further to encapsulate the agent-nanocluster combination and may be directed to a target by functionalizing the nanocluster with, e.g., aptamers and/or antibodies. Alternatively, an active ingredient may also be provided separately from the solid pharmaceutical composition, such as for co-administration. Such active ingredients can be any compound or mixture of compounds having therapeutic or other value when administered to an animal, particularly to a mammal, such as drugs, nutrients, cosmeceuticals, nutraceuticals, diagnostic agents, nutritional agents, and the like. The active agents described herein may be found in their native state, however, they will generally be provided in the form of a salt. The active agents described herein include their isomers, analogs and derivatives.

As used herein, the terms “targeting domain”, “targeting moiety”, or “targeting group” are used interchangeably and refer to all molecules capable of specifically binding to a particular target molecule and forming a bound complex as described above. Thus, the ligand and its corresponding target molecule form a specific binding pair.

The term “antibody”, as used herein, refers to an immunoglobulin molecule which is able to specifically bind to a specific epitope of an antigen. Antibodies can be intact immunoglobulins derived from natural sources, or from recombinant sources and can be immunoreactive portions of intact immunoglobulins. The antibodies in the present invention may exist in a variety of forms including, for example, polyclonal antibodies, monoclonal antibodies, multiple chain antibodies, intact immunoglobulins, synthetic antibodies, recombinant antibodies, intracellular antibodies (“intrabodies”), Fv, Fab, Fab′, F(ab)2 and F(ab′)2, as well as single chain antibodies (scFv), heavy chain antibodies, such as camelid antibodies, and humanized antibodies (Harlow et al., 1999, Using Antibodies: A Laboratory Manual, Cold Spring Harbor Laboratory Press, NY; Harlow et al., 1989, Antibodies: A Laboratory Manual, Cold Spring Harbor, New York; Houston et al., 1988, Proc. Natl. Acad. Sci. USA 85:5879-5883; Bird et al., 1988, Science 242:423-426).

The term “antibody fragment” refers to at least one portion of an intact antibody, or recombinant variants thereof, and refers to the antigen binding domain, e.g., an antigenic determining variable region of an intact antibody, that is sufficient to confer recognition and specific binding of the antibody fragment to a target, such as an antigen.

By the term “synthetic antibody” as used herein, is meant an antibody which is generated using recombinant DNA technology, such as, for example, an antibody expressed by a bacteriophage. The term should also be construed to mean an antibody which has been generated by the synthesis of a DNA molecule encoding the antibody and which DNA molecule expresses an antibody protein, or an amino acid sequence specifying the antibody, wherein the DNA or amino acid sequence has been obtained using synthetic DNA or amino acid sequence technology which is available and well known in the art.

A “humanized antibody” refers to a type of engineered antibody having its CDRs derived from a non-human donor immunoglobulin, the remaining immunoglobulin-derived parts of the molecule being derived from one (or more) human immunoglobulin(s). In addition, framework support residues may be altered to preserve binding affinity (see, e.g., 1989, Queen et al., Proc. Natl. Acad Sci USA, 86:10029-10032; 1991, Hodgson et al., Bio/Technology, 9:421). A suitable human acceptor antibody may be one selected from a conventional database, e.g., the KABAT database, Los Alamos database, and Swiss Protein database, by homology to the nucleotide and amino acid sequences of the donor antibody. A human antibody characterized by a homology to the framework regions of the donor antibody (on an amino acid basis) may be suitable to provide a heavy chain constant region and/or a heavy chain variable framework region for insertion of the donor CDRs. A suitable acceptor antibody capable of donating light chain constant or variable framework regions may be selected in a similar manner. It should be noted that the acceptor antibody heavy and light chains are not required to originate from the same acceptor antibody. The prior art describes several ways of producing such humanized antibodies (see for example EP-A-0239400 and EP-A-054951).

A “chimeric antibody” refers to a type of engineered antibody which contains a naturally-occurring variable region (light chain and heavy chains) derived from a donor antibody in association with light and heavy chain constant regions derived from an acceptor antibody.

The term “donor antibody” refers to an antibody (monoclonal, and/or recombinant) which contributes the amino acid sequences of its variable regions, CDRs, or other functional fragments or analogs thereof to a first immunoglobulin partner, so as to provide the altered immunoglobulin coding region and resulting expressed altered antibody with the antigenic specificity and neutralizing activity characteristic of the donor antibody.

The term “acceptor antibody” refers to an antibody (monoclonal and/or recombinant) heterologous to the donor antibody, which contributes all (or any portion, but in some embodiments all) of the amino acid sequences encoding its heavy and/or light chain framework regions and/or its heavy and/or light chain constant regions to the first immunoglobulin partner. In certain embodiments a human antibody is the acceptor antibody.

By the term “recombinant antibody” as used herein, is meant an antibody which is generated using recombinant DNA technology, such as, for example, an antibody expressed by a bacteriophage or yeast expression system. The term should also be construed to mean an antibody which has been generated by the synthesis of a DNA molecule encoding the antibody and which DNA molecule expresses an antibody protein, or an amino acid sequence specifying the antibody, wherein the DNA or amino acid sequence has been obtained using recombinant DNA or amino acid sequence technology which is available and well known in the art.

An “antibody heavy chain,” as used herein, refers to the larger of the two types of polypeptide chains present in antibody molecules in their naturally occurring conformations, and which normally determines the class to which the antibody belongs.

An “antibody light chain,” as used herein, refers to the smaller of the two types of polypeptide chains present in antibody molecules in their naturally occurring conformations. Kappa (x) and lambda (λ) light chains refer to the two major antibody light chain isotypes.

“CDRs” are defined as the complementarity determining region amino acid sequences of an antibody which are the hypervariable regions of immunoglobulin heavy and light chains. See, e.g., Kabat et al., Sequences of Proteins of Immunological Interest, 4th Ed., U.S. Department of Health and Human Services, National Institutes of Health (1987). There are three heavy chain and three light chain CDRs (or CDR regions) in the variable portion of an immunoglobulin. Thus, “CDRs” as used herein refers to all three heavy chain CDRs, or all three light chain CDRs (or both all heavy and all light chain CDRs, if appropriate). The structure and protein folding of the antibody may mean that other residues are considered part of the antigen binding region and would be understood to be so by a skilled person. See for example Chothia et al., (1989) Conformations of immunoglobulin hypervariable regions; Nature 342, p 877-883.

The terms “effective amount” and “pharmaceutically effective amount” refer to a sufficient amount of an agent to provide the desired biological result. That result can be reduction and/or alleviation of a sign, symptom, or cause of a disease or disorder, or any other desired alteration of a biological system. An appropriate effective amount in any individual case may be determined by one of ordinary skill in the art using routine experimentation.

A “therapeutically effective amount” refers to that amount which provides a therapeutic effect for a given condition and administration regimen. In particular, “therapeutically effective amount” means an amount that is effective to prevent, alleviate or ameliorate symptoms of the disease or prolong the survival of the subject being treated, which may be a human or non-human animal. Determination of a therapeutically effective amount is within the skill of the person skilled in the art.

As used herein, the term “stabilizers” refers to either, or both, primary particle and/or secondary stabilizers, which may be polymers or other small molecules. Non-limiting examples of primary particle and/or secondary stabilizers for use with the present invention include, e.g., starch, modified starch, and starch derivatives, gums, including but not limited to polymers, polypeptides, albumin, amino acids, thiols, amines, carboxylic acid and combinations or derivatives thereof. Other examples include xanthan gum, alginic acid, other alginates, benitoniite, veegum, agar, guar, locust bean gum, gum arabic, quince psyllium, flax seed, okra gum, arabinoglactin, pectin, tragacanth, scleroglucan, dextran, amylose, amylopectin, dextrin, etc., cross-linked polyvinylpyrrolidone, ion-exchange resins, potassium polymethacrylate, carrageenan (and derivatives), gum karaya and biosynthetic gum. Other examples of useful primary particle and/or secondary stabilizers include polymers such as: polycarbonates (linear polyesters of carbonic acid); microporous materials (bisphenol, a microporous poly(vinylchloride), micro-porous polyamides, microporous modacrylic copolymers, microporous styrene-acrylic and its copolymers); porous polysulfones, halogenated poly(vinylidene), polychloroethers, acetal polymers, polyesters prepared by esterification of a dicarboxylic acid or anhydride with an alkylene polyol, poly(alkylenesulfides), phenolics, polyesters, asymmetric porous polymers, cross-linked olefin polymers, hydrophilic microporous homopolymers, copolymers or interpolymers having a reduced bulk density, and other similar materials, poly(urethane), cross-linked chain-extended poly(urethane), poly(mides), poly(benzimidazoles), collodion, regenerated proteins, semi-solid cross-linked poly(vinylpyrrolidone).

The terms “coat,” “coated,” or “coating,” as used herein, refer to at least a partial coating of the organic liquid. One hundred percent coverage is not necessarily implied by these terms.

The term “label” when used herein refers to a detectable compound or composition that is conjugated directly or indirectly to a probe to generate a “labeled” probe. The label may be detectable by itself (e.g., radioisotope labels or fluorescent labels) or, in the case of an enzymatic label, may catalyze chemical alteration of a substrate compound or composition that is detectable (e.g., avidin-biotin). In some instances, primers can be labeled to detect a PCR product.

As used herein, the term “specific binding” refers to that binding which occurs between such paired species as enzyme/substrate, receptor/agonist, antibody/antigen, and lectin/carbohydrate which may be mediated by covalent or non-covalent interactions or a combination of covalent and non-covalent interactions. When the interaction of the two species produces a non-covalently bound complex, the binding which occurs is typically electrostatic, hydrogen-bonding, or the result of lipophilic interactions. Accordingly, “specific binding” occurs between a paired species where there is interaction between the two which produces a bound complex having the characteristics of an antibody/antigen or enzyme/substrate interaction. In particular, the specific binding is characterized by the binding of one member of a pair to a particular species and to no other species within the family of compounds to which the corresponding member of the binding member belongs. Thus, for example, an antibody preferably binds to a single epitope and to no other epitope within the family of proteins.

The term “specifically binds”, as used herein with respect to an antibody, is meant for an antibody which recognizes a specific antigen, but does not substantially recognize or bind other molecules in a sample. For example, an antibody that specifically binds to an antigen from one species may also bind to that antigen from one or more species. But, such cross-species reactivity does not itself alter the classification of an antibody as specific. In another example, an antibody that specifically binds to an antigen may also bind to different allelic forms of the antigen. However, such cross reactivity does not itself alter the classification of an antibody as specific. In some instances, the terms “specific binding” or “specifically binding,” can be used in reference to the interaction of an antibody, a protein, or a peptide with a second chemical species, to mean that the interaction is dependent upon the presence of a particular structure (e.g., an antigenic determinant or epitope) on the chemical species; for example, an antibody recognizes and binds to a specific protein structure rather than to proteins generally. If an antibody is specific for epitope “A”, the presence of a molecule containing epitope A (or free, unlabeled A), in a reaction containing labeled “A” and the antibody, will reduce the amount of labeled A bound to the antibody.

As used herein, the terms “peptide”, “polypeptide”, and “protein” are used interchangeably, and refer to a compound comprised of amino acid residues covalently linked by peptide bonds. A protein or peptide must contain at least two amino acids, and no limitation is placed on the maximum number of amino acids that can comprise a protein's or peptide's sequence. Polypeptides include any peptide or protein comprising two or more amino acids joined to each other by peptide bonds. As used herein, the term refers to both short chains, which also commonly are referred to in the art as peptides, oligopeptides and oligomers, for example, and to longer chains, which generally are referred to in the art as proteins, of which there are many types. “Polypeptides” include, for example, biologically active fragments, substantially homologous polypeptides, oligopeptides, homodimers, heterodimers, variants of polypeptides, modified polypeptides, derivatives, analogs, fusion proteins, among others. The polypeptides include natural peptides, recombinant peptides, synthetic peptides, or any combination thereof.

As used herein, “polynucleotide” includes cDNA, RNA, DNA/RNA hybrid, antisense RNA, ribozyme, genomic DNA, synthetic forms, and mixed polymers, both sense and antisense strands, and may be chemically or biochemically modified to contain non-natural or derivatized, synthetic, or semi-synthetic nucleotide bases. Also, contemplated are alterations of a wild type or synthetic gene, including but not limited to deletion, insertion, substitution of one or more nucleotides, or fusion to other polynucleotide sequences.

“Isolated” means altered or removed from the natural state. For example, a nucleic acid or a peptide naturally present in a living animal is not “isolated,” but the same nucleic acid or peptide partially or completely separated from the coexisting materials of its natural state is “isolated.” An isolated nucleic acid or protein can exist in substantially purified form, or can exist in a non-native environment such as, for example, a host cell.

The term “nutritional composition” may be a food product intended for human consumption, for example, a beverage, a drink, a bar, a snack, an ice cream, a dairy product, for example a chilled or a shelf-stable dairy product, a fermented dairy product, a drink, for example a milk-based drink, an infant formula, a growing-up milk, a confectionery product, a chocolate, a cereal product such as a breakfast cereal, a sauce, a soup, an instant drink, a frozen product intended for consumption after heating in a microwave or an oven, a ready-to-eat product, a fast food or a nutritional formula.

“Instructional material”, as that term is used herein, includes a publication, a recording, a diagram, or any other medium of expression which can be used to communicate the usefulness of the nucleic acid, peptide, and/or compound of the invention in the kit for identifying, diagnosing or alleviating or treating the various diseases or disorders recited herein. Optionally, or alternately, the instructional material may describe one or more methods of identifying, diagnosing or alleviating the diseases or disorders in a cell or a tissue of a subject. The instructional material of the kit may, for example, be affixed to a container that contains one or more components of the invention or be shipped together with a container that contains the one or more components of the invention. Alternatively, the instructional material may be shipped separately from the container with the intention that the recipient uses the instructional material and the components cooperatively.

Throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible sub-ranges as well as individual numerical values within that range. For example, description of a range such as from 1 to 6 should be considered to have specifically disclosed sub-ranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

### Nanoparticle

In one aspect, the present invention provides a nanoparticle comprising a dye. In another aspect, the present invention provides a nanoparticle comprising an aggregate of a dye. For example, in one embodiment, the nanoparticle comprises an indocyanine green (ICG) aggregate. In various aspects, the ICG is an indocyanine green J-aggregate (ICGJ). In some embodiments, the ICGJ is encapsulated in a nanoparticle. Thus, in one embodiment, the nanoparticle is a nanocapsule. In one embodiment, the nanoparticle is a nanocarrier. In one embodiment, the nanoparticle is a polymer vesicle. In one embodiment, the nanoparticle is a polymersome. In some embodiments, the ICGJ is encapsulated in a polymersome.

In various embodiments, the nanoparticle comprises at least one polymer and at least one dye. In one embodiment, the nanoparticle is a nanocapsule comprising at least one polymer that forms the nanocapsule, and at least one dye. In one embodiment, the nanoparticle is a polymersome comprising at least one polymer that forms the polymersome, and at least one dye. For example, in various embodiments, the nanoparticle or nanocapsule comprises ICGJ, polyethyleneimine (PEI), and poly(lactide-co-glycolic acid)-polyethylene glycol (PLGA-PEG); wherein the PLGA-PEG encapsulates the ICGJ and PEI. In one embodiment, the nanoparticle or nanocapsule comprises ICGJ, PEI, and poly(lactide-co-glycolic acid)-block-polyethylene glycol (PLGA-b-PEG); wherein the PLGA-b-PEG encapsulates the ICGJ and PEI.

In various embodiments, the nanoparticle or nanocapsule is a cationic polymer-free nanoparticle or nanocapsule. For example, in some embodiments, the nanoparticle or nanocapsule is a PEI-free nanoparticle or nanocapsule. In some embodiments, the PEI-free nanoparticle or nanocapsule comprises ICGJ, and PLGA-PEG; wherein the PLGA-PEG encapsulates the ICGJ. In one embodiment, the PEI-free nanoparticle or nanocapsule comprises ICGJ and PLGA-b-PEG; wherein the PLGA-b-PEG encapsulates the ICGJ.

In one embodiment, the nanoparticles encapsulate a dye. In one embodiment, the dye is a polymethine dye. In one embodiment, the polymethine dye can be a cyanine dye, hemicyanine dye, streptocyanine dye, mercocyanine dye, oxonol dye, styryl dye, diarylmethine dye, triarylmethine dye, rylenes, squaraines, and perylene hisandes and aza-analogs thereof. In one embodiment, the dye is a cyanine dye. Exemplary cyanine dyes include, but are not limited to, naphthalocyanine dyes, sulfonated indocyanines, indocyanine green, Cy3, Cy3.5, Cy5.5, and Cy7. In one embodiment, the dye is a merocyanine. Exemplary merocyanine dyes include, but are not limited to, pseudoisocyanine chloride and merocyanine I. In one embodiment, the dye is a squaraine. Exemplary squaraine dyes include, but are not limited to, squarylium dye III. In one embodiment, the dye is a rylene. Exemplary rylene dyes include, but are not limited to, bismide. In one embodiment, the dye is indocyanine green (ICG). In one embodiment, the dye is Congo Red. In one embodiment, the dye is IR783. In one embodiment, the dye is Briliant Blue G. In one embodiment, the dye is rhodamine 6G.

In one embodiment, the dye is an aggregate of a dye. In one embodiment, the polymethine dye is in an aggregate form. In one embodiment, the polymethine dye is a polymethine dye H-aggregate. In one embodiment, the polymethine dye is a polymethine dye J-aggregate. In one embodiment, formation of J-aggregates results in narrowing, red-shifting and enhancement of the absorption band. In one embodiment, formation of J-aggregates results in a decreased Stokes shift and enhanced fluorescence.

In one embodiment, the dye has an absorbance in the near infrared (NIR) range between about 650 and 1400 nm. In one embodiment, the dye absorbs has an absorbance in the NIR range between about 680 and 1100 nm. In one embodiment, the dye absorbs has an absorbance in the NIR range between about 700 and 950 nm. In one embodiment, the dye absorbs has an absorbance in the NIR range between about 715 and 950 nm. In one embodiment, the dye absorbs has an absorbance in the NIR range between about 790 and 895 nm.

In one embodiment, the nanoparticle comprises a dye has at least one absorption peak from about 650 to 1400 nm wavelength. In one embodiment, the nanoparticle encapsulating an aggregate of a dye has at least one absorption peak from about 650 to 1400 nm wavelength. In some embodiments, the absorption peak is from about 680 to 1100 nm wavelength. In some embodiments, the absorption peak is from about 715 to 950 nm wavelength. In some embodiments, the absorption peak from about 790 to 895 nm wavelength.

In one embodiment, the dye has a I₈₉₅/I₇₉₀ ratio in a range between about 0.1 and about 6.25. In one embodiment, the dye has a I₈₉₅/I₇₉₀ ratio in a range between about 1.4 and about 6.25. In one embodiment, the dye has a I₈₉₅/I₇₉₀ ratio in a range between about 1.6 and about 5.5. In one embodiment, the dye has a I₈₉₅/I₇₉₀ ratio of about 1.6. In one embodiment, the dye has a I₈₉₅/I₇₉₀ ratio of about 2.4. In one embodiment, the dye has a I₈₉₅/I₇₉₀ ratio of about 3.4. In one embodiment, the dye has a I₈₉₅/I₇₉₀ ratio of about 3.5. In one embodiment, the dye has a I₈₉₅/I₇₉₀ ratio of about 3.9. In one embodiment, the dye has a I₈₉₅/I₇₉₀ ratio of about 4.2. In one embodiment, the dye has a I₈₉₅/I₇₉₀ ratio of about 5.2. In one embodiment, the dye has a I₈₉₅/I₇₉₀ ratio of about 5.5. In one embodiment, the dye has a I₈₉₅/I₇₉₀ ratio of about 6.1. In one embodiment, the dye has a I₈₉₅/I₇₉₀ ratio of about 6.25.

Thus, in one embodiment, the nanoparticle has a I₈₉₅/I₇₉₀ ratio in a range between about 0.1 and about 6.25. In one embodiment, the nanoparticle has a I₈₉₅/I₇₉₀ ratio in a range between about 1.4 and about 6.25. In one embodiment, the nanoparticle has a I₈₉₅/I₇₉₀ ratio in a range between about 1.6 and about 5.5. In one embodiment, the nanoparticle has a I₈₉₅/I₇₉₀ ratio of about 1.6. In one embodiment, the nanoparticle has a I₈₉₅/I₇₉₀ ratio of about 2.4. In one embodiment, the nanoparticle has a I₈₉₅/I₇₉₀ ratio of about 3.4. In one embodiment, the nanoparticle has a I₈₉₅/I₇₉₀ ratio of about 3.5. In one embodiment, the nanoparticle has a I₈₉₅/I₇₉₀ ratio of about 3.9. In one embodiment, the nanoparticle has a I₈₉₅/I₇₉₀ ratio of about 4.2. In one embodiment, the nanoparticle has a I₈₉₅/I₇₉₀ ratio of about 5.2. In one embodiment, the nanoparticle has a I₈₉₅/I₇₉₀ ratio of about 5.5. In one embodiment, the nanoparticle has a I₈₉₅/I₇₉₀ ratio of about 6.1. In one embodiment, the nanoparticle has a I₈₉₅/I₇₉₀ ratio of about 6.25.

In one embodiment, the dye has a I₈₉₅/I₇₈₀ ratio in a range between about 0.1 and about 6.25. In one embodiment, the dye has a I₈₉₅/I₇₈₀ ratio in a range between about 1.4 and about 6.25. In one embodiment, the dye has a I₈₉₅/I₇₈₀ ratio in a range between about 1.6 and about 5.5. In one embodiment, the dye has a I₈₉₅/I₇₈₀ ratio of about 1.6. In one embodiment, the dye has a I₈₉₅/I₇₈₀ ratio of about 2.4. In one embodiment, the dye has a I₈₉₅/I₇₈₀ ratio of about 3.4. In one embodiment, the dye has a I₈₉₅/I₇₈₀ ratio of about 3.5. In one embodiment, the dye has a I₈₉₅/I₇₈₀ ratio of about 3.9. In one embodiment, the dye has a I₈₉₅/I₇₈₀ ratio of about 4.2. In one embodiment, the dye has a I₈₉₅/I₇₈₀ ratio of about 5.2. In one embodiment, the dye has a I₈₉₅/I₇₈₀ ratio of about 5.5. In one embodiment, the dye has a I₈₉₅/I₇₈₀ ratio of about 6.1. In one embodiment, the dye has a I₈₉₅/I₇₈₀ ratio of about 6.25.

Thus, in one embodiment, the nanoparticle has a I₈₉₅/I₇₈₀ ratio in a range between about 0.1 and about 6.25. In one embodiment, the nanoparticle has a I₈₉₅/I₇₈₀ ratio in a range between about 1.4 and about 6.25. In one embodiment, the nanoparticle has a I₈₉₅/I₇₈₀ ratio in a range between about 1.6 and about 5.5. In one embodiment, the nanoparticle has a I₈₉₅/I₇₈₀ ratio of about 1.6. In one embodiment, the nanoparticle has a I₈₉₅/I₇₈₀ ratio of about 2.4. In one embodiment, the nanoparticle has a I₈₉₅/I₇₈₀ ratio of about 3.4. In one embodiment, the nanoparticle has a I₈₉₅/I₇₈₀ ratio of about 3.5. In one embodiment, the nanoparticle has a I₈₉₅/I₇₈₀ ratio of about 3.9. In one embodiment, the nanoparticle has a I₈₉₅/I₇₈₀ ratio of about 4.2. In one embodiment, the nanoparticle has a I₈₉₅/I₇₈₀ ratio of about 5.2. In one embodiment, the nanoparticle has a I₈₉₅/I₇₈₀ ratio of about 5.5. In one embodiment, the nanoparticle has a I₈₉₅/I₇₈₀ ratio of about 6.1. In one embodiment, the nanoparticle has a I₈₉₅/I₇₈₀ ratio of about 6.25.

In one embodiment, the dye has a I₈₉₀/I₇₉₅ ratio in a range between about 0.1 and about 6.25. In one embodiment, the dye has a I₈₉₀/I₇₉₅ ratio in a range between about 1.4 and about 6.25. In one embodiment, the dye has a I₈₉₀/I₇₉₅ ratio in a range between about 1.6 and about 5.5. In one embodiment, the dye has a I₈₉₀/I₇₉₅ ratio of about 1.6. In one embodiment, the dye has a I₈₉₀/I₇₉₅ ratio of about 2.4. In one embodiment, the dye has a I₈₉₀/I₇₉₅ ratio of about 3.4. In one embodiment, the dye has a I₈₉₀/I₇₉₅ ratio of about 3.5. In one embodiment, the dye has a I₈₉₀/I₇₉₅ ratio of about 3.9. In one embodiment, the dye has a I₈₉₀/I₇₉₅ ratio of about 4.2. In one embodiment, the dye has a I₈₉₀/I₇₉₅ ratio of about 5.2. In one embodiment, the dye has a I₈₉₀/I₇₉₅ ratio of about 5.5. In one embodiment, the dye has a I₈₉₀/I₇₉₅ ratio of about 6.1. In one embodiment, the dye has a I₈₉₀/I₇₉₅ ratio of about 6.25.

Thus, in one embodiment, the nanoparticle has a I₈₉₀/I₇₉₅ ratio in a range between about 0.1 and about 6.25. In one embodiment, the nanoparticle has a I₈₉₀/I₇₉₅ ratio in a range between about 1.4 and about 6.25. In one embodiment, the nanoparticle has a I₈₉₀/I₇₉₅ ratio in a range between about 1.6 and about 5.5. In one embodiment, the nanoparticle has a I₈₉₀/I₇₉₅ ratio of about 1.6. In one embodiment, the nanoparticle has a I₈₉₀/I₇₉₅ ratio of about 2.4. In one embodiment, the nanoparticle has a I₈₉₀/I₇₉₅ ratio of about 3.4. In one embodiment, the nanoparticle has a I₈₉₀/I₇₉₅ ratio of about 3.5. In one embodiment, the nanoparticle has a I₈₉₀/I₇₉₅ ratio of about 3.9. In one embodiment, the nanoparticle has a I₈₉₀/I₇₉₅ ratio of about 4.2. In one embodiment, the nanoparticle has a I₈₉₀/I₇₉₅ ratio of about 5.2. In one embodiment, the nanoparticle has a I₈₉₀/I₇₉₅ ratio of about 5.5. In one embodiment, the nanoparticle has a I₈₉₀/I₇₉₅ ratio of about 6.1. In one embodiment, the nanoparticle has a I₈₉₀/I₇₉₅ ratio of about 6.25. In one embodiment, the dye aggregate has a particle size (e.g., average hydrodynamic diameter of the particle) of about 10 nm to about 100 nm. In one embodiment, the dye aggregate has a particle size (e.g., average hydrodynamic diameter of the particle) of about nm to about 60 nm.

Thus, in various embodiments, the nanoparticle has an average particle size (e.g., average hydrodynamic diameter of the nanoparticle) below about 250 nm. In some embodiments, the nanoparticle has a particle size (e.g., average hydrodynamic diameter of the nanoparticle) of about 50 nm to about 200 nm. In some embodiments, the nanoparticle has a particle size (e.g., average hydrodynamic diameter of the nanoparticle) of about 10 nm to about 150 nm. In some embodiments, the nanoparticle has a particle size (e.g., average hydrodynamic diameter of the nanoparticle) of about 70 nm to about 100 nm. In some embodiments, the nanoparticle is at least about 2.5 nm in diameter and not more than about 250 nm in diameter. In some embodiments, the nanoparticle is at least about 35 nm in diameter and not more than about 100 nm in diameter. In some embodiments, the nanoparticle is about 40 nm in diameter to from about 50 nm in diameter.

In one embodiment, the nanoparticle further comprises a cationic polymer. The cationic polymer may be a straight chain polymer (i.e., linear polymer) or a branched chain polymer (i.e., branched polymer), including hyperbranched polymers. In one embodiment the cationic polymer is a branched cationic polymer. In one embodiment, the cationic polymer is cross-linked. In one embodiment, the cationic polymer is a polyamine. In one embodiment, the cationic polymer has molecular weight of 5 kDa-3000 kDa. For example, in one embodiment, the cationic polymer has a molecular weight of 5 kDa-2000 kDa, 5 kDa-1500 kDa, 5 kDa-1000 kDa, 5 kDa-800 kDa, 5 kDa-500 kDa, 5 kDa-300 kDa or 5 kDa-200 kDa or 800 kDa-3000 kDa.

In one embodiment, the cationic polymer is a polyalkyleneimine (e.g., polyethyleneimine), polyallylamine, polyamidoamine, or poly(amino-co-ester). In one embodiment, the cationic polymer is polyethyleneimine (PEI), chitosan, poly(2-N,N-dimethylaminoethylmethacrylate), or poly-L-lysine. In one embodiment, the cationic polymer stabilizes the nanocapsule.

In various embodiments, the ratio of the dye to cationic polymer is less than about 0.99. In various embodiments, the ratio of the dye to cationic polymer is about 0.8. In various embodiments, the ratio of the dye to cationic polymer is about 0.7. In various embodiments, the ratio of the dye to cationic polymer is about 0.6. In various embodiments, the ratio of the dye to cationic polymer is about 0.5. In various embodiments, the ratio of the dye to cationic polymer is about 0.4. In various embodiments, the ratio of the dye to cationic polymer is about 0.3. In various embodiments, the ratio of the dye to cationic polymer is about 0.2. In various embodiments, the ratio of the dye to cationic polymer is about 0.1.

In one embodiment, the nanoparticle is any type of nanoparticle, including, but not limited to, nanocapsules, nanoclusters, nanovesicles, micelles, block copolymer micelles, lamellae shaped particles, polymersomes, dendrimers, and nano-size particles of various other small fabrications that are known to those in the art.

In one embodiment, the nanoparticle comprises one or more functional groups. In some embodiments, the functional group is an azide functional group, maleimide functional group, carboxyl functional group, amine functional group, hydrazine functional group, dibenzo-cyclooctyne functional group, or any combination thereof.

In one embodiment, the nanoparticle comprises one or more linker molecules. Examples of linker molecules include, but are not limited to, azide functionalized molecules, maleimide functionalized molecules, carboxyl functionalized molecules, amine functionalized molecules, hydrazine functionalized molecules, dibenzo-cyclooctyne functionalized molecules, or any combination thereof.

In one embodiment, the nanoparticle is a polymersome. Any polymersome known in the art may be utilized. Thus, in one embodiment, the nanoparticle comprises a homopolymer. In some embodiments, the nanoparticle comprises a block copolymer that is a triblock, tetrablock, pentablock, or at least six block copolymer. In some embodiments, the nanoparticle comprises poly(ethylene oxide) (PEO) block copolymer, poly(ethylethylene) (PEE), poly(butadiene) (PB or PBD), poly(styrene) (PS), poly(isoprene) (PI), PEI, poly(lactide-co-glycolic acid) (PLGA), biodegradable PLGA, polyethylene glycol (PEG), poly(lactide-co-glycolic acid)-polyethylene glycol (PLGA-PEG), poly(lactide-co-glycolic acid)-block-polyethylene glycol (PLGA-b-PEG), biodegradable PLGA-PEG, biodegradable PLGA-b-PEG, polyanhydride, polyanhydride-block-PEG copolymers, zwitterionic poly(carbobetaine), zwitterionic poly(sulfobetaine)-containing, zwitterionic poly(carbobetaine) and zwitterionic poly(sulfobetaine)-containing copolymers, poly(acrylic acid-co-distearin acrylate), poly(trimethylene carbonate)-block-poly(L-gluatamic acid), poly(ethylene glycol-block-L-aspartic acid), poly(2-hydroxyethyl-co-octadecyl aspartamide), poly(ethylene glycol-co-trimethylene carbonate-co-caprolactone, polypropylene oxide block copolymers, polyethylene oxide-block-polypropylene oxide copolymers, or any combination thereof.

In some embodiments, the nanoparticle comprises poly(ε-caprolactone) (PCL) diblock co-polymer. In some embodiments, the nanoparticle comprises poly(ethylene oxide)-block-poly(ε-caprolactone) (PEO-b-PCL) based diblock copolymers. In some embodiments, the nanoparticle is derived from the coupling of poly(lactic acid), poly(glycolide), poly(lactic-coglycolic acid) and/or or poly(3-hydroxybutyrate) with PEO. In some embodiments, the nanoparticle comprises PLGA. In some embodiments, the nanoparticle comprises PEG. In one embodiment, the nanoparticle comprises poly(lactide-co-glycolic acid)-polyethylene glycol (PLGA-PEG). For example, in some embodiments, a PLGA-PEG polymersome encapsulates the ICGJ and optionally PEI.

In one embodiment, the nanoparticle is resistant to protein opsonization. In one embodiment, the nanoparticle is a coated nanoparticle.

In one embodiment, the nanoparticle further comprises a targeting domain. In one aspect, the nanoparticle further comprises a targeting domain attached to the surface of the nanoparticle. In some embodiments, the targeting domain is bound to an exterior surface of the nanoparticle and recognizes a particular site of interest in a subject. In one embodiment, the targeting domain binds to at least one associated with a disease or a disorder. In one embodiment, the targeting domain binds to at least one cancer cell. In one embodiment, the targeting domain binds to at least one tumor cell. In one embodiment, the targeting domain binds to at least one cancer biomarker. Examples of cancer biomarkers include, but are not limited to tumor antigens, tumor-specific antigens and tumor-associated antigens, tissue differentiation antigens, mutant protein antigens, epidermal-growth factor receptor (EGFR), oncogenic viral antigens (e.g., alphafetoprotein (AFP) and carcinoembryonic antigen (CEA)), cancer-testis antigens (e.g., CTAG1B and MAGEA1), vascular or stromal specific antigens, epithelial tumor antigen (ETA), tyrosinase, melanoma-associated antigen (MAGE), abnormal products of ras, p53, MUC-1; and tumor markers, such as AFP, carcinoma antigen (CA), CA15-3, CA27-29, CA19-9, CA-125, calcitonin, calretinin, CEA, CD34, CD99MIC 2, CD117, chromogranin, chromosomers 3, 7, 17, and 9p21, cytokeratin (e.g., TPA, TPS, Cyfra21-1, etc.), desmin, epithelial membrane antigen (EMA), factor VIII, CD31, FL1, glial fibrillary acidic protein (GFAP), gross cystic disease fluid protein (GCDFP-15), human melanoma black 45 (HMB-45), human chorionic gonadotropin (hCG), immunoglobulin, inhibin, keratin, Lactate dehydrogenase (LDH), lymphocyte marker, melanoma antigen recognized by T cells 1 (MART-1), Melan-A, myoblast determination protein 1 (Myo D1), muscle-specific actin (MSA), neurofilament, neuron-specific enolase (NSE), placental alkaline phosphatase (PLAP), prostate-specific antigen (PSA), prostatic acid phosphatase (PAP), protein tyrosine phosphatase receptor type C (PTPRC or CD45), S100 protein, smooth muscle actin (SMA), synaptophysin, thymidine kinase, thyroglobulin (Tg), thyroid transcription factor-1 (TTF-1), tumor M2-PK, and vimentin. In one embodiment, the targeting domain binds to at least one epidermal-growth factor receptor (EGFR).

In various embodiments, the targeting domain is an antibody, an antibody fragment, a peptide sequence, aptamer, folate, a ligand, a gene component, or any combination thereof. Examples of targeting domains include, but are not limited to antibodies, lymphokines, cytokines, receptor proteins such as CD4 and CD8, solubilized receptor proteins such as soluble CD4, hormones, growth factors, peptidomimetics, synthetic ligands, and the like which specifically bind desired target cells, and nucleic acids which bind corresponding nucleic acids through base pair complementarity. Targeting domains of particular interest include peptidomimetics, peptides, aptamers, folates, ligands, gene components, antibodies (e.g., monoclonal antibodies, polyclonal antibodies, recombinant antibodies, human antibodies, humanized antibodies, single domain antibodies (nanobodies), etc.) and antibody fragments (e.g., the Fab′ fragment).

In certain embodiments, the targeting domain specifically binds to a tumor-associated antigen (TAA) or tumor specific antigen (TSA). Cellular targets include tissue specific cell surface molecules, for targeting to specific sites of interest (e.g., neural cells, liver cells, bone marrow cells, kidney cells, pancreatic cells, muscle cells, and the like).

Methods of making and using antibodies are well known in the art. For example, polyclonal antibodies useful in the present invention are generated by immunizing rabbits according to standard immunological techniques well-known in the art (see, e.g., Harlow et al., 1988, In: Antibodies, A Laboratory Manual, Cold Spring Harbor, N.Y.). Such techniques include immunizing an animal with a chimeric protein comprising a portion of another protein such as a maltose binding protein or glutathione (GSH) tag polypeptide portion, and/or a moiety such that the antigenic protein of interest is rendered immunogenic (e.g., an antigen of interest conjugated with keyhole limpet hemocyanin, KLH) and a portion comprising the respective antigenic protein amino acid residues.

However, the invention should not be construed as being limited solely to methods and compositions including these antibodies or to these portions of the antigens. Rather, the invention should be construed to include other antibodies, as that term is defined elsewhere herein, to antigens, or portions thereof. Further, the present invention should be construed to encompass antibodies, inter alia, which bind to the specific antigens of interest.

One skilled in the art would appreciate, based upon the disclosure provided herein, that the antibody can specifically bind with any portion of an antigen target, which can be used to generate antibodies specific therefor. However, the present invention is not limited to using the full-length protein as an immunogen. Rather, the present invention includes using an immunogenic portion of the protein to produce an antibody that specifically binds with a specific antigen. That is, the invention includes immunizing an animal using an immunogenic portion, or antigenic determinant, of the antigen.

The antibodies can be produced by immunizing an animal such as, but not limited to, a rabbit, a mouse or a camel, with an antigenic protein of the invention, or a portion thereof, by immunizing an animal using a protein comprising at least a portion of the antigen, or a fusion protein including a tag polypeptide portion comprising, for example, a maltose binding protein tag polypeptide portion, covalently linked with a portion comprising the appropriate amino acid residues. One skilled in the art would appreciate, based upon the disclosure provided herein, that smaller fragments of these proteins can also be used to produce antibodies that specifically bind the antigen of interest.

Once armed with the sequence of a specific antigen of interest and the detailed analysis localizing the various conserved and non-conserved domains of the protein, the skilled artisan would understand, based upon the disclosure provided herein, how to obtain antibodies specific for the various portions of the antigen using methods well-known in the art or to be developed.

Further, the skilled artisan, based upon the disclosure provided herein, would appreciate that using a non-conserved immunogenic portion can produce antibodies specific for the non-conserved region thereby producing antibodies that do not cross-react with other proteins which can share one or more conserved portions. Thus, one skilled in the art would appreciate, based upon the disclosure provided herein, that the non-conserved regions of an antigen of interest can be used to produce antibodies that are specific only for that antigen and do not cross-react non-specifically with other proteins.

The invention encompasses monoclonal, synthetic antibodies, and the like. One skilled in the art would understand, based upon the disclosure provided herein, that the crucial feature of the antibody of the invention is that the antibody bind specifically with an antigen of interest. That is, the antibody of the invention recognizes an antigen of interest or a fragment thereof (e.g., an immunogenic portion or antigenic determinant thereof).

The skilled artisan would appreciate, based upon the disclosure provided herein, that present invention includes use of a single antibody recognizing a single antigenic epitope but that the invention is not limited to use of a single antibody. Instead, the invention encompasses use of at least one antibody where the antibodies can be directed to the same or different antigenic protein epitopes.

The generation of polyclonal antibodies is accomplished by inoculating the desired animal with the antigen and isolating antibodies which specifically bind the antigen therefrom using standard antibody production methods, such as those described in, for example, Harlow et al. (1988, In: Antibodies, A Laboratory Manual, Cold Spring Harbor, N.Y.).

Monoclonal antibodies directed against full length or peptide fragments of a protein or peptide may be prepared using any well-known monoclonal antibody preparation procedures, such as those described, for example, in Harlow et al. (1988, In: Antibodies, A Laboratory Manual, Cold Spring Harbor, N.Y.) and in Tuszynski et al. (1988, Blood, 72:109-115). Quantities of the desired peptide may also be synthesized using chemical synthesis technology. Alternatively, DNA encoding the desired peptide may be cloned and expressed from an appropriate promoter sequence in cells suitable for the generation of large quantities of peptide. Monoclonal antibodies directed against the peptide are generated from mice immunized with the peptide using standard procedures as referenced herein.

Nucleic acid encoding the monoclonal antibody obtained using the procedures described herein may be cloned and sequenced using technology which is available in the art, and is described, for example, in Wright et al. (1992, Critical Rev. Immunol. 12:125-168), and the references cited therein. Further, the antibody of the invention may be “humanized” using the technology described in, for example, Wright et al., and in the references cited therein, and in Gu et al. (1997, Thrombosis and Hematocyst 77:755-759), and other methods of humanizing antibodies well-known in the art or to be developed.

In some embodiments, a non-human antibody is humanized, where specific sequences or regions of the antibody are modified to increase similarity to an antibody naturally produced in a human or fragment thereof. A humanized antibody can be produced using a variety of techniques known in the art, including but not limited to, CDR-grafting (see, e.g., European Patent No. EP 239,400; International Publication No. WO 91/09967; and U.S. Pat. Nos. 5,530,101, and 5,585,089, each of which is incorporated herein in its entirety by reference), veneering or resurfacing (see, e.g., European Patent Nos. EP 592,106 and EP 519,596; Padlan, 1991, Molecular Immunology, 28(4/5):489-498; Studnicka et al., 1994, Protein Engineering, 7(6):805-814; and Roguska et al., 1994, PNAS, 91:969-973, each of which is incorporated herein by its entirety by reference), chain shuffling (see, e.g., U.S. Pat. No. 5,565,332, which is incorporated herein in its entirety by reference), and techniques disclosed in, e.g., U.S. Patent Application Publication No. US2005/0042664, U.S. Patent Application Publication No. US2005/0048617, U.S. Pat. Nos. 6,407,213, 5,766,886, International Publication No. WO 9317105, Tan et al., J. Immunol., 169:1119-25 (2002), Caldas et al., Protein Eng., 13(5):353-60 (2000), Morea et al., Methods, 20(3):267-79 (2000), Baca et al., J. Biol. Chem., 272(16):10678-84 (1997), Roguska et al., Protein Eng., 9(10):895-904 (1996), Couto et al., Cancer Res., 55 (23 Supp):5973s-5977s (1995), Couto et al., Cancer Res., (1995), Sandhu J S, Gene, 150(2):409-10 (1994), and Pedersen et al., J. Mol. Biol., 235(3):959-73 (1994), each of which is incorporated herein in its entirety by reference. Often, framework residues in the framework regions will be substituted with the corresponding residue from the CDR donor antibody to alter, for example improve, antigen binding. These framework substitutions are identified by methods well-known in the art, e.g., by modeling of the interactions of the CDR and framework residues to identify framework residues important for antigen binding and sequence comparison to identify unusual framework residues at particular positions. (See, e.g., Queen et al., U.S. Pat. No. 5,585,089; and Riechmann et al., 1988, Nature, 332:323, which are incorporated herein by reference in their entireties.)

In one embodiment, the antibody fragment provided herein is a single chain variable fragment (scFv). In various embodiments, the antibodies of the invention may exist in a variety of other forms including, for example, Fv, Fab, and (Fab′) 2, as well as bi-functional (i.e. bi-specific) hybrid antibodies (e.g., Lanzavecchia et al., Eur. J. Immunol. 17, 105 (1987)). In some embodiments, the antibodies and fragments thereof of the invention bind a cell bearing antigen, TCR, and/or BCR with wild-type or enhanced affinity. In some embodiments, the antibodies and fragments thereof of the invention bind a T cell bearing TCR with wild-type or enhanced affinity. In some embodiments, the antibodies and fragments thereof of the invention bind a B cell bearing BCR with wild-type or enhanced affinity. In various embodiments, a human scFv may also be derived from a yeast display library.

ScFvs can be prepared according to method known in the art (see, for example, Bird et al., (1988) Science 242:423-426 and Huston et al., (1988) Proc. Natl. Acad. Sci. USA 85:5879-5883). ScFv molecules can be produced by linking VH and VL regions together using flexible polypeptide linkers. The scFv molecules comprise flexible polypeptide linker (e.g., a Ser-Gly linker) with an optimized length and/or amino acid composition. The flexible polypeptide linker length can greatly affect how the variable regions of an scFv fold and interact. In fact, if a short polypeptide linker is employed (e.g., between 5-10 amino acids, intrachain folding is prevented. Interchain folding is also required to bring the two variable regions together to form a functional epitope binding site. For examples of linker orientation and size see, e.g., Hollinger et al. 1993 Proc Natl Acad. Sci. U.S.A. 90:6444-6448, U.S. Patent Application Publication Nos. 2005/0100543, 2005/0175606, 2007/0014794, and PCT publication Nos. WO2006/020258 and WO2007/024715, is incorporated herein by reference.

The scFv can comprise a polypeptide linker sequence of at least 1, 2, 3, 4, 5, 6, 7, 8, 9, 10, 11, 12, 13, 14, 15, 16, 17, 18, 19, 20, 25, 30, 35, 40, 45, 50, or more amino acid residues between its VL and VH regions. The flexible polypeptide linker sequence may comprise any naturally occurring amino acid. In some embodiments, the flexible polypeptide linker sequence comprises amino acids glycine and serine. In another embodiment, the flexible polypeptide linker sequence comprises sets of glycine and serine repeats such as (Gly4Ser)n, where n is a positive integer equal to or greater than 1. In one embodiment, the flexible polypeptide linkers include, but are not limited to, (Gly4Ser)4 or (Gly4Ser)3. Variation in the flexible polypeptide linker length may retain or enhance activity, giving rise to superior efficacy in activity studies.

In one embodiment, the targeting domain is bound directly to the nanoparticle. In one embodiment, the targeting domain is bound directly to the surface of the nanoparticle. In one embodiment, the targeting domain is bound directly to the cationic polymer. In one embodiment, the targeting domain is bound to the nanoparticle using a linking molecule. In one embodiment, the targeting domain is bound to the surface of the nanoparticle using a linking molecule. In one embodiment, the targeting domain is bound to the cationic polymer using a linking molecule. The linking molecules useful in the compositions and methods of the present disclosure may be any molecule capable of binding to both the coating material used in the compositions and methods of the present disclosure and the targeting domains used in the compositions and methods of the present disclosure. In certain embodiments, the linking molecule may be a hydrophilic polymer. Examples of linking molecules include, but are not limited to, poly(ethylene glycol) and its derivatives, azide compounds, maleimide compounds, hydzrazine compounds, dibenzo-cyclooctyne (DBCO) compounds, dithiol compounds, dithiol compounds with hydrazide and/or carboxylic functionality, or single thiols and/or amines or their derivatives.

In certain embodiments, the linking molecule and the targeting domain may be bound by one or more covalent bonds. In certain embodiments, the linking molecule, in addition to linking the targeting domain and the coating material, may impart certain benefits upon the compositions of the present disclosure, including, but not limited to, improved hydrophilicity and stability in solution, reduced immunogenic responses upon introduction of the compositions of the present disclosure into a subject, increased circulation time of the compositions of the present disclosure when introduced into the bloodstream of a subject. The choice of a linking molecule may depend upon, among other things, the targeting domain chosen and the subject into which the compositions of the present invention are to be introduced. One of ordinary skill in the art, with the benefit of this disclosure, will recognize additional suitable linking molecules. Such linking molecules are considered to be within the spirit of the present disclosure.

In certain embodiments, the targeting domain may recognize a particular ligand or receptor present in a desired cell and/or tissue type when introduced into a subject. In certain embodiments, the targeting domain may be an antibody that recognizes such a particular ligand or receptor. The use of antibody fragments may also be suitable in the compositions of the present disclosure. The choice of a targeting domain may depend upon, among other things, the cell and/or tissue type into which an at least partial increase in uptake of the compositions of the present disclosure is desired, as well as particular ligand(s) present in such cell and/or tissue types.

In certain embodiments, the targeting domain may be chosen, among other things, to at least partially increase the uptake of the compositions of the present disclosure into a desired cell and/or tissue type when introduced into a subject. In certain embodiments, the targeting domain may be a moiety that recognizes a molecule which is present in higher amounts in an abnormal form of a tissue when compared to a normal form of the same tissue (i.e., the molecule is “up-regulated” in the abnormal form of the tissue).

In some embodiments, the suitable targeting domain may be a peptide sequence, DNA fragment, aptamer, RNA, folate, polymer, etc. One of ordinary skill in the art, with the benefit of this disclosure, will recognize other targeting domains that may be useful in the compositions of the present disclosure. Such targeting domains are considered to be within the spirit of the present disclosure.

In one aspect, the nanoparticle comprises one or more antibodies. In one embodiment, the nanoparticle is bound to the antibody. In one embodiment, the PLGA-PEG-encapsulated ICGJ is conjugated with at least one antibody. In one embodiment, the PLGA-PEG-encapsulated PEI is conjugated with at least one antibody. In one embodiment, the PLGA-PEG is conjugated with at least one antibody. In one embodiment, the antibody selectively binds cells associated with a disease or a disorder. In one embodiment, the antibody selectively binds cancer cells. In one embodiment, the antibody selectively binds tumor cells. In one embodiment, the antibody is specific for EGFR. In various embodiments, the antibody is a monoclonal antibody (mAb). In one embodiment, the mAb is a grafted mAb.

In one aspect of the invention, the nanoparticle further comprises one or more therapeutic agents. In one embodiment, the nanoparticle is bound to the therapeutic agent. In some embodiments, the nanoparticle comprises at least one polymer that forms polymersome, which encapsulates at least one dye and at least one therapeutic agent. In one embodiment, the nanoparticle comprises at least one polymer that forms polymersome, which encapsulates at least one dye, at least one additional polymer, and at least one therapeutic agent. In one embodiment, the therapeutic agent is a hydrophobic therapeutic agent. In one embodiment, the therapeutic agent is a hydrophilic therapeutic agent. Examples of such therapeutic agents include, but are not limited to, one or more drugs, proteins, amino acids, peptides, antibodies, antibiotics, small molecules, anti-cancer agents, chemotherapeutic agents, immunomodulatory agents, RNA molecules, siRNA molecules, DNA molecules, gene editing agents, gene-silencing agents, CRISPR-associated agents (e.g., guide RNA molecules, endonucleases, and variants thereof), medical imaging agents, therapeutic moieties, one or more non-therapeutic moieties or a combination to target cancer or atherosclerosis, selected from folic acid, peptides, proteins, aptamers, antibodies, siRNA, poorly water soluble drugs, anti-cancer drugs, antibiotics, analgesics, vaccines, anticonvulsants; anti-diabetic agents, antifungal agents, antineoplastic agents, anti-parkinsonian agents, anti-rheumatic agents, appetite suppressants, biological response modifiers, cardiovascular agents, central nervous system stimulants, contraceptive agents, dietary supplements, vitamins, minerals, lipids, saccharides, metals, amino acids (and precursors), nucleic acids and precursors, contrast agents, diagnostic agents, dopamine receptor agonists, erectile dysfunction agents, fertility agents, gastrointestinal agents, hormones, immunomodulators, antihypercalcemia agents, mast cell stabilizers, muscle relaxants, nutritional agents, ophthalmic agents, osteoporosis agents, psychotherapeutic agents, parasympathomimetic agents, parasympatholytic agents, respiratory agents, sedative hypnotic agents, skin and mucous membrane agents, smoking cessation agents, steroids, sympatholytic agents, urinary tract agents, uterine relaxants, vaginal agents, vasodilator, anti-hypertensive, hyperthyroids, anti-hyperthyroids, anti-asthmatics and vertigo agents, or any combinations thereof.

In one embodiment, the therapeutic agent is one or more non-therapeutic moieties. In some embodiments, the nanoparticle comprises one or more therapeutic moieties, one or more non-therapeutic moieties, or any combination thereof. In one embodiment, the therapeutic moiety targets cancer. In some embodiments, the composition comprises folic acid, peptides, proteins, aptamers, antibodies, small RNA molecules, miRNA, shRNA, siRNA, poorly water-soluble therapeutic agents, anti-cancer agents, or any combinations thereof.

In one embodiment, the therapeutic agent may be an anti-cancer agent. Any suitable anti-cancer agent may be used in the compositions and methods of the present disclosure. The selection of a suitable anti-cancer agent may depend upon, among other things, the type of cancer to be treated and the nanoparticle compositions of the present disclosure. In certain embodiments, the anti-cancer agent may be effective for treating one or more of pancreatic cancer, esophageal cancer, rectal cancer, colon cancer, prostate cancer, kidney cancer, liver cancer, breast cancer, ovarian cancer, and stomach cancer. Examples of anti-cancer agents include, but is not limited to, chemotherapeutic agents, antiproliferative agents, anti-tumor agents, checkpoint inhibitors, and anti-angiogenic agents. For example, in one embodiment, the anti-cancer agent is gemcitabine, doxorubicin, 5-Fu, tyrosine kinase inhibitors, sorafenib, trametinib, rapamycin, fulvestrant, ezalutamide, or paclitaxel.

Chemotherapeutic agents include cytotoxic agents (e.g., 5-fluorouracil, cisplatin, carboplatin, methotrexate, daunorubicin, doxorubicin, vincristine, vinblastine, oxorubicin, carmustine (BCNU), lomustine (CCNU), cytarabine USP, cyclophosphamide, estramucine phosphate sodium, altretamine, hydroxyurea, ifosfamide, procarbazine, mitomycin, busulfan, cyclophosphamide, mitoxantrone, carboplatin, cisplatin, interferon alfa-2a recombinant, paclitaxel, teniposide, and streptozoci), cytotoxic alkylating agents (e.g., busulfan, chlorambucil, cyclophosphamide, melphalan, or ethylesulfonic acid), alkylating agents (e.g., asaley, AZQ, BCNU, busulfan, bisulphan, carboxyphthalatoplatinum, CBDCA, CCNU, CHIP, chlorambucil, chlorozotocin, cis-platinum, clomesone, cyanomorpholinodoxorubicin, cyclodisone, cyclophosphamide, dianhydrogalactitol, fluorodopan, hepsulfam, hycanthone, iphosphamide, melphalan, methyl CCNU, mitomycin C, mitozolamide, nitrogen mustard, PCNU, piperazine, piperazinedione, pipobroman, porfiromycin, spirohydantoin mustard, streptozotocin, teroxirone, tetraplatin, thiotepa, triethylenemelamine, uracil nitrogen mustard, and Yoshi-864), antimitotic agents (e.g., allocolchicine, Halichondrin M, colchicine, colchicine derivatives, dolastatin 10, maytansine, rhizoxin, paclitaxel derivatives, paclitaxel, thiocolchicine, trityl cysteine, vinblastine sulfate, and vincristine sulfate), plant alkaloids (e.g., actinomycin D, bleomycin, L-asparaginase, idarubicin, vinblastine sulfate, vincristine sulfate, mitramycin, mitomycin, daunorubicin, VP-16-213, VM-26, navelbine and taxotere), biologicals (e.g., alpha interferon, BCG, G-CSF, GM-CSF, and interleukin-2), topoisomerase I inhibitors (e.g., camptothecin, camptothecin derivatives, and morpholinodoxorubicin), topoisomerase II inhibitors (e.g., mitoxantron, amonafide, m-AMSA, anthrapyrazole derivatives, pyrazoloacridine, bisantrene HCL, daunorubicin, deoxydoxorubicin, menogaril, N,N-dibenzyl daunomycin, oxanthrazole, rubidazone, VM-26 and VP-16), and synthetics (e.g., hydroxyurea, procarbazine, o,p′-DDD, dacarbazine, CCNU, BCNU, cis-diamminedichloroplatimun, mitoxantrone, CBDCA, levamisole, hexamethylmelamine, all-trans retinoic acid, gliadel and porfimer sodium).

Antiproliferative agents are compounds that decrease the proliferation of cells. Antiproliferative agents include alkylating agents, antimetabolites, enzymes, biological response modifiers, miscellaneous agents, hormones and antagonists, androgen inhibitors (e.g., flutamide and leuprolide acetate), antiestrogens (e.g., tamoxifen citrate and analogs thereof, toremifene, droloxifene and roloxifene), Additional examples of specific antiproliferative agents include, but are not limited to levamisole, gallium nitrate, granisetron, sargramostim strontium-89 chloride, filgrastim, pilocarpine, dexrazoxane, and ondansetron.

The inhibitors of the invention can be administered alone or in combination with other anti-tumor agents, including cytotoxic/antineoplastic agents and anti-angiogenic agents. Cytotoxic/anti-neoplastic agents are defined as agents which attack and kill cancer cells. Some cytotoxic/anti-neoplastic agents are alkylating agents, which alkylate the genetic material in tumor cells, e.g., cis-platin, cyclophosphamide, nitrogen mustard, trimethylene thiophosphoramide, carmustine, busulfan, chlorambucil, belustine, uracil mustard, chlomaphazin, and dacabazine. Other cytotoxic/anti-neoplastic agents are antimetabolites for tumor cells, e.g., cytosine arabinoside, fluorouracil, methotrexate, mercaptopuirine, azathioprime, and procarbazine. Other cytotoxic/anti-neoplastic agents are antibiotics, e.g., doxorubicin, bleomycin, dactinomycin, daunorubicin, mithramycin, mitomycin, mytomycin C, and daunomycin. There are numerous liposomal formulations commercially available for these compounds. Still other cytotoxic/anti-neoplastic agents are mitotic inhibitors (vinca alkaloids). These include vincristine, vinblastine and etoposide. Miscellaneous cytotoxic/anti-neoplastic agents include taxol and its derivatives, L-asparaginase, anti-tumor antibodies, dacarbazine, azacytidine, amsacrine, melphalan, VM-26, ifosfamide, mitoxantrone, and vindesine.

Anti-angiogenic agents are well known to those of skill in the art. Suitable anti-angiogenic agents for use in the methods and compositions of the present disclosure include anti-VEGF antibodies, including humanized and chimeric antibodies, anti-VEGF aptamers and antisense oligonucleotides. Other known inhibitors of angiogenesis include angiostatin, endostatin, interferons, interleukin 1 (including alpha and beta) interleukin 12, retinoic acid, and tissue inhibitors of metalloproteinase-1 and -2. (TIMP-1 and -2). Small molecules, including topoisomerases such as razoxane, a topoisomerase II inhibitor with anti-angiogenic activity, can also be used.

Other anti-cancer agents that can be used in combination with the disclosed compounds include, but are not limited to: acivicin; aclarubicin; acodazole hydrochloride; acronine; adozelesin; aldesleukin; altretamine; ambomycin; ametantrone acetate; aminoglutethimide; amsacrine; anastrozole; anthramycin; asparaginase; asperlin; azacitidine; azetepa; azotomycin; batimastat; benzodepa; bicalutamide; bisantrene hydrochloride; bisnafide dimesylate; bizelesin; bleomycin sulfate; brequinar sodium; bropirimine; busulfan; cactinomycin; calusterone; caracemide; carbetimer; carboplatin; carmustine; carubicin hydrochloride; carzelesin; cedefingol; chlorambucil; cirolemycin; cisplatin; cladribine; crisnatol mesylate; cyclophosphamide; cytarabine; dacarbazine; dactinomycin; daunorubicin hydrochloride; decitabine; dexormaplatin; dezaguanine; dezaguanine mesylate; diaziquone; docetaxel; doxorubicin; doxorubicin hydrochloride; droloxifene; droloxifene citrate; dromostanolone propionate; duazomycin; edatrexate; eflornithine hydrochloride; elsamitrucin; enloplatin; enpromate; epipropidine; epirubicin hydrochloride; erbulozole; esorubicin hydrochloride; estramustine; estramustine phosphate sodium; etanidazole; etoposide; etoposide phosphate; etoprine; fadrozole hydrochloride; fazarabine; fenretinide; floxuridine; fludarabine phosphate; fluorouracil; fluorocitabine; fosquidone; fostriecin sodium; gemcitabine; gemcitabine hydrochloride; hydroxyurea; idarubicin hydrochloride; ifosfamide; ilmofosine; interleukin II (including recombinant interleukin II, or rIL2), interferon alfa-2a; interferon alfa-2b; interferon alfa-n1; interferon alfa-n3; interferon beta-I a; interferon gamma-I b; iproplatin; irinotecan hydrochloride; lanreotide acetate; letrozole; leuprolide acetate; liarozole hydrochloride; lometrexol sodium; lomustine; losoxantrone hydrochloride; masoprocol; maytansine; mechlorethamine hydrochloride; megestrol acetate; melengestrol acetate; melphalan; menogaril; mercaptopurine; methotrexate; methotrexate sodium; metoprine; meturedepa; mitindomide; mitocarcin; mitocromin; mitogillin; mitomalcin; mitomycin; mitosper; mitotane; mitoxantrone hydrochloride; mycophenolic acid; nocodazole; nogalamycin; ormaplatin; oxisuran; paclitaxel; pegaspargase; peliomycin; pentamustine; peplomycin sulfate; perfosfamide; pipobroman; piposulfan; piroxantrone hydrochloride; plicamycin; plomestane; porfimer sodium; porfiromycin; prednimustine; procarbazine hydrochloride; puromycin; puromycin hydrochloride; pyrazofurin; riboprine; rogletimide; safingol; safingol hydrochloride; semustine; simtrazene; sparfosate sodium; sparsomycin; spirogermanium hydrochloride; spiromustine; spiroplatin; streptonigrin; streptozocin; sulofenur; talisomycin; tecogalan sodium; tegafur; teloxantrone hydrochloride; temoporfin; teniposide; teroxirone; testolactone; thiamiprine; thioguanine; thiotepa; tiazofurin; tirapazamine; toremifene citrate; trestolone acetate; triciribine phosphate; trimetrexate; trimetrexate glucuronate; triptorelin; tubulozole hydrochloride; uracil mustard; uredepa; vapreotide; verteporfin; vinblastine sulfate; vincristine sulfate; vindesine; vindesine sulfate; vinepidine sulfate; vinglycinate sulfate; vinleurosine sulfate; vinorelbine tartrate; vinrosidine sulfate; vinzolidine sulfate; vorozole; zeniplatin; zinostatin; zorubicin hydrochloride. Other anti-cancer drugs include, but are not limited to: 20-epi-1,25 dihydroxyvitamin D3; 5-ethynyluracil; abiraterone; aclarubicin; acylfulvene; adecypenol; adozelesin; aldesleukin; ALL-TK antagonists; altretamine; ambamustine; amidox; amifostine; aminolevulinic acid; amrubicin; amsacrine; anagrelide; anastrozole; andrographolide; angiogenesis inhibitors; antagonist D; antagonist G; antarelix; anti-dorsalizing morphogenetic protein-1; antiandrogen, prostatic carcinoma; antiestrogen; antineoplaston; antisense oligonucleotides; aphidicolin glycinate; apoptosis gene modulators; apoptosis regulators; apurinic acid; ara-CDP-DL-PTBA; arginine deaminase; asulacrine; atamestane; atrimustine; axinastatin 1; axinastatin 2; axinastatin 3; azasetron; azatoxin; azatyrosine; baccatin III derivatives; balanol; batimastat; BCR/ABL antagonists; benzochlorins; benzoylstaurosporine; beta lactam derivatives; beta-alethine; betaclamycin B; betulinic acid; bFGF inhibitor; bicalutamide; bisantrene; bisaziridinylspermine; bisnafide; bistratene A; bizelesin; breflate; bropirimine; budotitane; buthionine sulfoximine; calcipotriol; calphostin C; camptothecin derivatives; canarypox IL-2; capecitabine; carboxamide-amino-triazole; carboxyamidotriazole; CaRest M3; CARN 700; cartilage derived inhibitor; carzelesin; casein kinase inhibitors (ICOS); castanospermine; cecropin B; cetrorelix; chlorins; chloroquinoxaline sulfonamide; cicaprost; cis-porphyrin; cladribine; clomifene analogues; clotrimazole; collismycin A; collismycin B; combretastatin A4; combretastatin analogue; conagenin; crambescidin 816; crisnatol; cryptophycin 8; cryptophycin A derivatives; curacin A; cyclopentanthraquinones; cycloplatam; cypemycin; cytarabine ocfosfate; cytolytic factor; cytostatin; dacliximab; decitabine; dehydrodidemnin B; deslorelin; dexamethasone; dexifosfamide; dexrazoxane; dexverapamil; diaziquone; didemnin B; didox; diethylnorspermine; dihydro-5-azacytidine; dihydrotaxol, 9-; dioxamycin; diphenyl spiromustine; docetaxel; docosanol; dolasetron; doxifluridine; droloxifene; dronabinol; duocarmycin SA; ebselen; ecomustine; edelfosine; edrecolomab; eflornithine; elemene; emitefur; epirubicin; epristeride; estramustine analogue; estrogen agonists; estrogen antagonists; etanidazole; etoposide phosphate; exemestane; fadrozole; fazarabine; fenretinide; filgrastim; finasteride; flavopiridol; flezelastine; fluasterone; fludarabine; fluorodaunorunicin hydrochloride; forfenimex; formestane; fostriecin; fotemustine; gadolinium texaphyrin; gallium nitrate; galocitabine; ganirelix; gelatinase inhibitors; gemcitabine; glutathione inhibitors; hepsulfam; heregulin; hexamethylene bisacetamide; hypericin; ibandronic acid; idarubicin; idoxifene; idramantone; ilmofosine; ilomastat; imidazoacridones; imiquimod; immunostimulant peptides; insulin-like growth factor-1 receptor inhibitor; interferon agonists; interferons; interleukins; iobenguane; iododoxorubicin; ipomeanol, 4-; iroplact; irsogladine; isobengazole; isohomohalicondrin B; itasetron; jasplakinolide; kahalalide F; lamellarin-N triacetate; lanreotide; leinamycin; lenograstim; lentinan sulfate; leptolstatin; letrozole; leukemia inhibiting factor; leukocyte alpha interferon; leuprolide+estrogen+progesterone; leuprorelin; levamisole; liarozole; linear polyamine analogue; lipophilic disaccharide peptide; lipophilic platinum compounds; lissoclinamide 7; lobaplatin; lombricine; lometrexol; lonidamine; losoxantrone; lovastatin; loxoribine; lurtotecan; lutetium texaphyrin; lysofylline; lytic peptides; maitansine; mannostatin A; marimastat; masoprocol; maspin; matrilysin inhibitors; matrix metalloproteinase inhibitors; menogaril; merbarone; meterelin; methioninase; metoclopramide; MIF inhibitor; mifepristone; miltefosine; mirimostim; mismatched double stranded RNA; mitoguazone; mitolactol; mitomycin analogues; mitonafide; mitotoxin fibroblast growth factor-saporin; mitoxantrone; mofarotene; molgramostim; monoclonal antibody, human chorionic gonadotrophin; monophosphoryl lipid A+myobacterium cell wall sk; mopidamol; multiple drug resistance gene inhibitor; multiple tumor suppressor 1-based therapy; mustard anticancer agent; mycaperoxide B; mycobacterial cell wall extract; myriaporone; N-acetyldinaline; N-substituted benzamides; nafarelin; nagrestip; naloxone+pentazocine; napavin; naphterpin; nartograstim; nedaplatin; nemorubicin; neridronic acid; neutral endopeptidase; nilutamide; nisamycin; nitric oxide modulators; nitroxide antioxidant; nitrullyn; O6-benzylguanine; octreotide; okicenone; oligonucleotides; onapristone; ondansetron; ondansetron; oracin; oral cytokine inducer; ormaplatin; osaterone; oxaliplatin; oxaunomycin; paclitaxel; paclitaxel analogues; paclitaxel derivatives; palauamine; palmitoylrhizoxin; pamidronic acid; panaxytriol; panomifene; parabactin; pazelliptine; pegaspargase; peldesine; pentosan polysulfate sodium; pentostatin; pentrozole; perflubron; perfosfamide; perillyl alcohol; phenazinomycin; phenylacetate; phosphatase inhibitors; picibanil; pilocarpine hydrochloride; pirarubicin; piritrexim; placetin A; placetin B; plasminogen activator inhibitor; platinum complex; platinum compounds; platinum-triamine complex; porfimer sodium; porfiromycin; prednisone; propyl bis-acridone; prostaglandin J2; proteasome inhibitors; protein A-based immune modulator; protein kinase C inhibitor; protein kinase C inhibitors, microalgal; protein tyrosine phosphatase inhibitors; purine nucleoside phosphorylase inhibitors; purpurins; pyrazoloacridine; pyridoxylated hemoglobin polyoxyethylene conjugate; raf antagonists; raltitrexed; ramosetron; ras farnesyl protein transferase inhibitors; ras inhibitors; ras-GAP inhibitor; retelliptine demethylated; rhenium Re 186 etidronate; rhizoxin; ribozymes; RII retinamide; rogletimide; rohitukine; romurtide; roquinimex; rubiginone B1; ruboxyl; safingol; saintopin; SarCNU; sarcophytol A; sargramostim; Sdi 1 mimetics; semustine; senescence derived inhibitor 1; sense oligonucleotides; signal transduction inhibitors; signal transduction modulators; single chain antigen binding protein; sizofuran; sobuzoxane; sodium borocaptate; sodium phenylacetate; solverol; somatomedin binding protein; sonermin; sparfosic acid; spicamycin D; spiromustine; splenopentin; spongistatin 1; squalamine; stem cell inhibitor; stem-cell division inhibitors; stipiamide; stromelysin inhibitors; sulfinosine; superactive vasoactive intestinal peptide antagonist; suradista; suramin; swainsonine; synthetic glycosaminoglycans; tallimustine; tamoxifen methiodide; tauromustine; tazarotene; tecogalan sodium; tegafur; tellurapyrylium; telomerase inhibitors; temoporfin; temozolomide; teniposide; tetrachlorodecaoxide; tetrazomine; thaliblastine; thiocoraline; thrombopoietin; thrombopoietin mimetic; thymalfasin; thymopoietin receptor agonist; thymotrinan; thyroid stimulating hormone; tin ethyl etiopurpurin; tirapazamine; titanocene bichloride; topsentin; toremifene; totipotent stem cell factor; translation inhibitors; tretinoin; triacetyluridine; triciribine; trimetrexate; triptorelin; tropisetron; turosteride; tyrosine kinase inhibitors; tyrphostins; UBC inhibitors; ubenimex; urogenital sinus-derived growth inhibitory factor; urokinase receptor antagonists; vapreotide; variolin B; vector system, erythrocyte gene therapy; velaresol; veramine; verdins; verteporfin; vinorelbine; vinxaltine; vitaxin; vorozole; zanoterone; zeniplatin; zilascorb; and zinostatin stimalamer. In one embodiment, the anti-cancer drug is 5-fluorouracil, taxol, or leucovorin.

In some embodiments, the anti-cancer agent may be a prodrug form of an anti-cancer agent. As used herein, the term “prodrug form” and its derivatives is used to refer to a drug that has been chemically modified to add and/or remove one or more substituents in such a manner that, upon introduction of the prodrug form into a subject, such a modification may be reversed by naturally occurring processes, thus reproducing the drug. The use of a prodrug form of an anti-cancer agent in the compositions, among other things, may increase the concentration of the anti-cancer agent in the compositions of the present disclosure. In certain embodiments, an anti-cancer agent may be chemically modified with an alkyl or acyl group or some form of lipid. The selection of such a chemical modification, including the substituent(s) to add and/or remove to create the prodrug, may depend upon a number of factors including, but not limited to, the particular drug and the desired properties of the prodrug. One of ordinary skill in the art, with the benefit of this disclosure, will recognize suitable chemical modifications.

In some embodiments, the nanoparticle further comprises one or more gene components, such as siRNA or therapeutic DNA fragments. In some embodiments, the gene component is encapsulated in the nanoparticle. In some embodiments, the gene component is on the surface of the nanoparticle, for example, attached to or within the coating material.

In some embodiments, the nanoparticle further comprises a biocompatible metal. Examples of biocompatible metals include, but are not limited to, copper, copper sulfide, iron oxide, cobalt and noble metals, such as gold and/or silver. One of ordinary skill in the art will be able to select of a suitable type of nanoparticle taking into consideration at least the type of imaging and/or therapy to be performed.

In one aspect of the invention, the nanoparticle is a biodegradable nanoparticle. In one embodiment, the nanoparticle is biodegradable nanocapsule. In one embodiment, the nanoparticle is a biodegradable polymer vesicle. In one embodiment, the nanoparticle is a biodegradable polymersome. In one embodiment, the polymersome comprises biodegradable PLGA-PEG. In one embodiment, the polymersome comprises biodegradable PLGA-b-PEG.

The present invention also provides various compositions comprising the nanoparticles of the present invention. In some embodiments, the nanoparticle composition is a nanocapsule, nanocarrier, contrast agent, nanocluster composition, nanocarrier composition, contrast agent composition, or any combination thereof. In one embodiment, the nanocluster composition is a biodegradable nanocluster composition. In one embodiment, the nanocluster composition is a medical biodegradable nanocluster composition.

In various embodiments, the nanoparticle composition comprises one or more nanoparticles of the present invention and one or more alcohols. Examples of such alcohols include, but are not limited to, polyvinyl alcohol (PVA), methyl alcohol, ethyl alcohol, propyl alcohol, butyl alcohol, or any combination thereof. For example, in one embodiment, in one embodiment, the nanoparticle composition comprises one or more nanoparticle and PVA. In one embodiment, in one embodiment, the nanoparticle composition comprises one or more PEI-free nanoparticle and PVA. In one embodiment, in one embodiment, the nanoparticle composition comprises one or more nanoparticle and ethyl alcohol. In one embodiment, in one embodiment, the nanoparticle composition comprises one or more PEI-free nanoparticle and ethyl alcohol.

In some embodiments, the nanoparticle composition comprises alcohol in a range between about 0.1% and about 99.9%. In some embodiments, the nanoparticle composition comprises alcohol in a range between about 1% and about 50%. In some embodiments, the nanoparticle composition comprises alcohol in a range between about 10% and about 50%. In some embodiments, the nanoparticle composition comprises alcohol in a range between about 10% and about 40%. In some embodiments, the nanoparticle composition comprises alcohol in a range between about 10% and about 30%. In some embodiments, the nanoparticle composition comprises alcohol in a range between about 10% and about 20%. In some embodiments, the nanoparticle composition comprises about 10% alcohol. For example, in one embodiment, the nanoparticle composition comprises about 10% ethyl alcohol.

In various aspects, the nanoparticle composition comprises: one or more nanoparticles of the present invention and one or more stabilizers. In various embodiments, the stabilizer to nanoparticle weight ratio is less than 50%. In one embodiment, the stabilizer comprises a biocompatible polymer. Examples of stabilizers include, but are not limited to, biocompatible polymer, a biodegradable polymer, a multifunctional linker, starch, modified starch, and starch derivatives, gums, including but not limited to polymers, polypeptides, albumin, amino acids, alcohols (e.g., PVA, ethyl alcohol, etc.), thiols, amines, carboxylic acid and combinations or derivatives thereof, citric acid, xanthan gum, alginic acid, other alginates, benitoniite, veegum, agar, guar, locust bean gum, gum arabic, quince psyllium, flax seed, okra gum, arabinoglactin, pectin, tragacanth, scleroglucan, dextran, amylose, amylopectin, dextrin, etc., cross-linked polyvinylpyrrolidone, ion-exchange resins, potassium polymethacrylate, carrageenan (and derivatives), gum karaya and biosynthetic gum, polycarbonates (linear polyesters of carbonic acid); microporous materials (bisphenol, a microporous poly(vinylchloride), micro-porous polyamides, microporous modacrylic copolymers, microporous styrene-acrylic and its copolymers); porous polysulfones, halogenated poly(vinylidene), polychloroethers, acetal polymers, polyesters prepared by esterification of a dicarboxylic acid or anhydride with an alkylene polyol, poly(alkylenesulfides), phenolics, polyesters, asymmetric porous polymers, cross-linked olefin polymers, hydrophilic microporous homopolymers, copolymers or interpolymers having a reduced bulk density, and other similar materials, poly(urethane), cross-linked chain-extended poly(urethane), poly(imides), poly(benzimidazoles), collodion, regenerated proteins, semi-solid cross-linked poly(vinylpyrrolidone), monomeric, dimeric, oligomeric or long-chain, copolymers, block polymers, block co-polymers, polymers, PEG, dextran, modified dextran, polyvinylalcohol, polyvinylpyrollidone, polyacrylates, polymethacrylates, polyanhydrides, polypeptides, albumin, alginates, amino acids, thiols, amines and carboxylic acids or combinations thereof.

In various aspects, the nanoparticle of the present invention may be delivered to a cell or biological tissue of interest by a red-blood cell-hitchhiking methods that are well-known to those of skill in the art, and such methods are incorporated herein by reference. Examples of such red-blood cell-hitchhiking methods are described in Brenner et al., 2018, Nature Commun., 9:2684.

In various embodiments, the clearance, excretion rates, and pathways of the nanoparticles can be predetermined by the size (e.g., average diameter) of the nanoparticles and physiochemical properties of capping ligands and templating polymers. This approach provides a flexible platform for designing and validation of various types of nanoparticles for safe clinical use. For example, different types of nanomaterials can be clustered together providing multiplexing opportunities for synthesis of multifunctional/multimodal nanoparticles.

In some embodiments, further applications include therapeutic agent encapsulation inside the nanoparticles with controlled release that can be triggered by one of the following stimulus: polymer degradation in tumor microenvironment, enzyme sensitive polymers, or by an external stimulus, such as NIR light. In some embodiments, the nanoparticle of the present disclosure may be “remotely triggered” by applying energy or a pH change to the nanoparticle composition. In some embodiments, the energy, such as an electromagnetic field, magnetic field, optical methods (e.g., ultraviolet (UV) irradiation, UV-vis-NIR irradiation, infrared (IR) irradiation, NIR irradiation), or specific radiofrequencies, may be applied to biological tissue thereby causing the release of a therapeutic agent from the nanoparticle further comprising the therapeutic agent. For example, in one embodiment, a NIR radiation is applied to a nanoparticle comprising a therapeutic agent, wherein the NIR radiation heats the nanoparticle causing the release of the therapeutic agent from the nanoparticle. In another embodiment, a magnetic field is applied to a nanoparticle comprising a therapeutic agent and a magnetic component, wherein the magnetic field heats the nanoparticle causing the release of the therapeutic agent from the nanoparticle. In another embodiment, a pH change is applied to a nanoparticle comprising a therapeutic agent, wherein the pH change causes the release of the therapeutic agent from the nanoparticle. In some embodiments, this may provide a clinician the ability to control and visualize drug therapy noninvasively.

### Contrast Agent

The present invention also provides a contrast agent based on the nanoparticles described herein for imaging. For example, in one embodiment, the contrast agent is useful for imaging of EGFR, metallo-proteases 2 and 9, oncoproteins associated with HPV 16 induced carcinogenesis, actin, cells associated with a disease or disorder, cancer biomarkers, cancer cells, and tumor cells. In various embodiments, the contrast agent absorbs in the NIR spectrum (e.g., from around about 650 nm to around about 1400 nm; from around about 680 nm to around about 1100 nm; from about 790 to about 895 nm; etc.).

In various embodiments, the contrast agent has a I₈₉₅/I₇₉₀ ratio in a range between about 0.1 and about 6.25. In one embodiment, the n contrast agent has a I₈₉₅/I₇₉₀ ratio in a range between about 1.4 and about 6.25. In one embodiment, the n contrast agent has a I₈₉₅/I₇₉₀ ratio in a range between about 1.6 and about 5.5. In one embodiment, the contrast agent has a I₈₉₅/I₇₉₀ ratio of about 1.6. In one embodiment, the contrast agent has a I₈₉₅/I₇₉₀ ratio of about 2.4. In one embodiment, the contrast agent has a I₈₉₅/I₇₉₀ ratio of about 3.4. In one embodiment, the contrast agent has a I₈₉₅/I₇₉₀ ratio of about 3.5. In one embodiment, the contrast agent has a I₈₉₅/I₇₉₀ ratio of about 3.9. In one embodiment, the contrast agent has a I₈₉₅/I₇₉₀ ratio of about 4.2. In one embodiment, the contrast agent has a I₈₉₅/I₇₉₀ ratio of about 5.2. In one embodiment, the contrast agent has a I₈₉₅/I₇₉₀ ratio of about 5.5. In one embodiment, the contrast agent has a I₈₉₅/I₇₉₀ ratio of about 6.1. In one embodiment, the contrast agent has a I₈₉₅/I₇₉₀ ratio of about 6.25.

In various embodiments, the contrast agent has a I₈₉₅/I₇₈₀ ratio in a range between about 0.1 and about 6.25. In one embodiment, the n contrast agent has a I₈₉₅/I₇₈₀ ratio in a range between about 1.4 and about 6.25. In one embodiment, the n contrast agent has a I₈₉₅/I₇₈₀ ratio in a range between about 1.6 and about 5.5. In one embodiment, the contrast agent has a I₈₉₅/I₇₈₀ ratio of about 1.6. In one embodiment, the contrast agent has a I₈₉₅/I₇₈₀ ratio of about 2.4. In one embodiment, the contrast agent has a I₈₉₅/I₇₈₀ ratio of about 3.4. In one embodiment, the contrast agent has a I₈₉₅/I₇₈₀ ratio of about 3.5. In one embodiment, the contrast agent has a I₈₉₅/I₇₈₀ ratio of about 3.9. In one embodiment, the contrast agent has a I₈₉₅/I₇₈₀ ratio of about 4.2. In one embodiment, the contrast agent has a I₈₉₅/I₇₈₀ ratio of about 5.2. In one embodiment, the contrast agent has a I₈₉₅/I₇₈₀ ratio of about 5.5. In one embodiment, the contrast agent has a I₈₉₅/I₇₈₀ ratio of about 6.1. In one embodiment, the contrast agent has a I₈₉₅/I₇₈₀ ratio of about 6.25.

In one embodiment, the contrast agent has a I₈₉₀/I₇₉₅ ratio in a range between about 0.1 and about 6.25. In one embodiment, the contrast agent has a I₈₉₀/I₇₉₅ ratio in a range between about 1.4 and about 6.25. In one embodiment, the contrast agent has a I₈₉₀/I₇₉₅ ratio in a range between about 1.6 and about 5.5. In one embodiment, the contrast agent has a I₈₉₀/I₇₉₅ ratio of about 1.6. In one embodiment, the contrast agent has a I₈₉₀/I₇₉₅ ratio of about 2.4. In one embodiment, the contrast agent has a I₈₉₀/I₇₉₅ ratio of about 3.4. In one embodiment, the contrast agent has a I₈₉₀/I₇₉₅ ratio of about 3.5. In one embodiment, the contrast agent has a I₈₉₀/I₇₉₅ ratio of about 3.9. In one embodiment, the contrast agent has a I₈₉₀/I₇₉₅ ratio of about 4.2. In one embodiment, the contrast agent has a I₈₉₀/I₇₉₅ ratio of about 5.2. In one embodiment, the contrast agent has a I₈₉₀/I₇₉₅ ratio of about 5.5. In one embodiment, the contrast agent has a I₈₉₀/I₇₉₅ ratio of about 6.1. In one embodiment, the contrast agent has a I₈₉₀/I₇₉₅ ratio of about 6.25.

In some embodiments, biologically active agents may be added to the nanoparticles for molecular specific optoacoustic imaging of cancer cells.

In one aspect, the contrast agent comprises the nanoparticle of the present invention and a pharmaceutically acceptable excipient. In various embodiments, the contrast agent further comprises an organic component. In one embodiment, the organic component is a lipid composition. In one embodiment, the organic component comprises one or more targeting domains. In one embodiment, the nanoparticle is complexed with a lipid. In one embodiment, the lipid comprises at least 50% diacetylene phospholipid. In one embodiment, the lipid further comprises at least one targeting lipid. In one embodiment, the targeting lipid comprises a targeting domain specific for a cancer cell. In some embodiments, the nanoparticles of the present invention are bound to the surface of the organic component. Examples of such organic surface include, but are not limited to, a protein surface, lipid surface, or a combination thereof.

In various aspects, the contrast agent is a photoacoustic imaging (PAI) contrast agent, ultrasound imaging contrast agent, optical imaging contrast agent, magnetic resonance imaging contrast agent, computed tomography contrast agent, thermal imaging contrast agent, nuclear imaging contrast agent, magnetomotive imaging enhancement contrast agent, fluorescence imaging contrast agent, and any combination thereof.

In various aspects, the nanoparticle composition of the present invention acts as a contrast agent for continuous wave photoacoustic imaging, combined photoacoustic and ultrasound imaging, magnetomotive imaging, optical coherent tomography, magnetic resonance imaging, computed tomography, nuclear imaging modalities, or any combination thereof. Furthermore, when the nanoparticle composition contains magnetic iron oxide and/or cobalt nanoparticles, it may be used in microwave ablation therapy and magnetomotive imaging enhancement.

In some embodiments, the nanoparticle is used as contrast enhancement for optical imaging methods, such as optical coherence tomography, magnetic resonance imaging, computed tomography, fluorescence imaging, and photoacoustic imaging (for example, through mechanisms of vaporization and thermal expansion).

In various embodiments, the contrast agents comprise a self-assembled aggregates comprising the nanoparticles of the present invention. In some embodiments, the aggregates are cross-linked.

To obtain selectivity, the contrast agent may be passively or actively targeted to regions of diagnostic interest, such as organs, vessels, sites of disease, tumorous tissue, or a specific organism in a subject. In active targeting, the contrast agents may be attached to biological recognition agents to allow them to accumulate in or to be selectively retained by or to be slowly eliminated from certain parts of the body, such as specific organs, parts of organs, bodily structures and disease structures and lesions. Active targeting is defined as a modification of biodistribution using chemical groups that will associate with species present in the desired tissue or organism to effectively decrease the rate of loss of contrast agent from the specific tissue or organism.

Active targeting of a contrast agent can be considered as localization through modification of biodistribution of the contrast agent by means of a targeting domain that is attached to or incorporated into the contrast agent. The targeting domain can associate or bind with one or more receptor species present in the tissue or organism of diagnostic interest. This binding will effectively decrease the rate of loss of contrast agent from the specific tissue or organism of diagnostic interest. In such cases, the contrast agent can be modified synthetically to incorporate the targeting domain. Targeted contrast agents can localize because of binding between the ligand and the targeted receptor. Alternatively, contrast agents can distribute by passive biodistribution, i.e., by passive targeting, into diseased tissues of interest such as tumors. Thus, even without synthetic manipulation to incorporate a targeting domain that can bind to a receptor site, passively targeted contrast agents can accumulate in a diseased tissue or in specific locations in the subject, such as the liver. The present invention comprises use of a contrast agent that is linked to a targeting domain that has an affinity for binding to a receptor. Preferably the receptor is located on the surface of a diseased or disease-causing cell in a human or animal subject.

The contrast agents are formulated in a pharmaceutically acceptable excipient, such as wetting agents, buffers, disintegrants, binders, fillers, flavoring agents and liquid carrier media such as sterile water, water/ethanol etc. The contrast agent should be suitable for administration either by injection or inhalation or catheterization or instillation or transdermal introduction into any of the various body cavities including the alimentary canal, the vagina, the rectum, the bladder, the ureter, the urethra, the mouth, etc. For oral administration, the pH of the composition is preferably in the acid range (e.g., 2 to 7) and buffers or pH adjusting agents may be used. The contrast media may be formulated in conventional pharmaceutical administration forms, such as tablets, capsules, powders, solutions, dispersion, syrups, suppositories etc.

### Method of Preparation

The present invention also describes a method of forming nanoparticles described herein comprising the steps of: (i) forming one or more aggregates of a dye; and (ii) encapsulating one or more dye aggregates in the polymer shell. In various embodiments, the method of forming nanoparticles is a cationic polymer-free (e.g., PEI-free) method of forming nanoparticles. For example, in some embodiments, the method of forming a nanoparticle comprises the steps of: (i) forming one or more ICG aggregates (e.g., ICGJ, etc.); and (ii) encapsulating the ICG aggregate complexes in a polymer shell comprising PLGA-b-PEG.

In various embodiments, the method further comprises (iii) heating the nanoparticles. In some embodiments, the nanoparticles are heated in a range between about 30° C. to about 100° C. In some embodiments, the nanoparticles are heated in a range between about ° C. to about 70° C. In some embodiments, the nanoparticles are heated in a range between about 50° C. to about 70° C. In one embodiment, the nanoparticles are heated at about 65° C.

The present invention also describes a method of forming nanoparticles described herein comprising the steps of: (i) forming one or more aggregates of a dye; (ii) adsorbing a layer of one or more polymers onto the surface of the dye aggregate to form a polymer shell; and (iii) encapsulating one or more dye aggregates in the polymer shell. In various embodiments, the method of forming nanoparticles comprises the steps of: (i) forming one or more aggregates of a dye; (ii) complexing one or more polymers with the aggregate; (iii) encapsulating one or more polymer-aggregate complexes in a polymer shell comprising of a second polymer. For example, in some embodiments, the method of forming a nanoparticle comprises the steps of: (i) forming one or more ICG aggregates (e.g., ICGJ, etc.); (ii) complexing one or more polymers (e.g., PLGA-b-PEG, etc.) with the ICG aggregate; and (iii) encapsulating the PLGA-b-PEG-ICG aggregate complexes in a polymer shell comprising PLGA-b-PEG.

In various embodiments, the method further comprises (iv) heating the nanoparticles. In some embodiments, the nanoparticles are heated in a range between about 30° C. to about 100° C. In some embodiments, the nanoparticles are heated in a range between about 40° C. to about 70° C. In some embodiments, the nanoparticles are heated in a range between about 50° C. to about 70° C. In one embodiment, the nanoparticles are heated at about 65° C.

In some embodiments, the method of forming nanoparticles comprises the steps of: (i) forming one or more aggregates of a dye, wherein the aggregates comprise an anionic surface; (ii) complexing one or more cationic polymers with the anionic surface of the aggregate; (iii) encapsulating one or more cationic polymer-anionic aggregate complexes in a polymer shell comprising of a second polymer. For example, in some embodiments, the method of forming a nanoparticle comprises the steps of: (i) forming one or more ICG aggregates (e.g., ICGJ, etc.), wherein the ICG comprises an anionic surface; (ii) complexing one or more cationic polymers (e.g., PEI, etc.) onto the anionic surface of the ICG aggregate; and (iii) encapsulating the PEI-ICG aggregate complexes in a polymer shell comprising PLGA-b-PEG.

In various embodiments, adsorbing a layer of one or more polymers onto the surface of the dye aggregate prevents the dissociation of the dye aggregate. In some embodiments, adsorbing a layer of one or more polymers onto the surface of the dye aggregate reduces the dissociation of the dye aggregate. In various embodiments, adsorbing a layer of one or more polymers onto the surface of the dye aggregate prevents the decomposition of the dye aggregate. In some embodiments, adsorbing a layer of one or more polymers onto the surface of the dye aggregate reduces the decomposition of the dye aggregate.

In various aspects, the present invention also describes a method of forming nanoparticles (e.g., polymersomes) described herein comprising the steps of: (i) forming a W1 water phase/O oil phase emulsion, wherein the W1 water phase comprises one or more aggregates of a dye and the O oil phase comprises one or more polymers; and (ii) adding an outer W2 water phase to form W1/O/W2 double emulsion, wherein the outer W2 phase comprises one or more sacrificial aggregates of the dye. For example, in some embodiments, the method of forming a nanoparticle (e.g., polymersome) comprises the steps of: (i) forming a W1 water phase/O oil phase emulsion, wherein the W1 water phase comprises one or more ICG aggregates (e.g., ICGJ) and the O oil phase comprises PLGA-PEG; and (ii) adding an outer W2 water phase to form W1/O/W2 double emulsion, wherein the outer W2 phase comprises one or more sacrificial ICG aggregates (e.g., ICGJ). In some embodiments, the method of forming a nanoparticle (e.g., polymersome) comprises the steps of: (i) forming a W1 water phase/O oil phase emulsion, wherein the W1 water phase comprises one or more ICG aggregates (e.g., ICGJ) encapsulated by PEI, and wherein the O oil phase comprises PLGA-PEG; and (ii) adding an outer W2 water phase to form W1/O/W2 double emulsion, wherein the outer W2 phase comprises one or more sacrificial ICG aggregates (e.g., ICGJ).

In various embodiments, the method of forming nanoparticles is a cationic polymer-free (e.g., PEI-free) method of forming nanoparticles. For example, in some embodiments, the cationic polymer-free (e.g., PEI-free) method of forming a nanoparticle (e.g., polymersome) comprises the steps of: (i) forming a W1 water phase/O oil phase emulsion, wherein the W1 water phase comprises one or more ICG aggregates (e.g., ICGJ) and the O oil phase comprises PLGA-PEG; and (ii) adding an outer W2 water phase to form W1/O/W2 double emulsion, wherein the outer W2 phase comprises one or more sacrificial ICG aggregates (e.g., ICGJ). In some embodiments, the cationic polymer-free (e.g., PEI-free) method of forming a nanoparticle (e.g., polymersome) comprises the steps of: (i) forming a W1 water phase/O oil phase emulsion, wherein the W1 water phase comprises one or more ICG aggregates (e.g., ICGJ) encapsulated by PEI, and wherein the O oil phase comprises PLGA-PEG; and (ii) adding an outer W2 water phase to form W1/O/W2 double emulsion, wherein the outer W2 phase comprises one or more sacrificial ICG aggregates (e.g., ICGJ).

In various embodiments, the method further comprises (iii) heating the nanoparticles. In some embodiments, the nanoparticles are heated in a range between about 30° C. to about 100° C. In some embodiments, the nanoparticles are heated in a range between about 40° C. to about 70° C. In some embodiments, the nanoparticles are heated in a range between about 50° C. to about 70° C. In one embodiment, the nanoparticles are heated at about 65° C.

In various embodiments, the sacrificial dye aggregate in the outer W2 water phase prevents the dissociation of the nanoparticle (e.g., polymersome). In some embodiments, the sacrificial dye aggregate in the outer W2 water phase reduces the dissociation of the nanoparticle (e.g., polymersome). In various embodiments, the sacrificial dye aggregate in the outer W2 water phase prevents the decomposition of the nanoparticle (e.g., polymersome). In some embodiments, the sacrificial dye aggregate in the outer W2 water phase reduces the decomposition of the nanoparticle (e.g., polymersome).

In one example, the method of forming cationic polymer-free nanoparticles (e.g., cationic polymer-free polymersomes, PEI-free nanoparticles, etc.) comprises using a concentration of about 50 mg/mL to about 500 mg/mL of the dye aggregate in the W1 phase. For example, in one embodiment, the concentration of the dye aggregate in the W1 water phase is about 100 mg/ml, about 125 mg/ml, about 150 mg/ml, about 175 mg/ml, or about 200 mg/mL or more. In one embodiment, no dye or dye aggregate is used in the W2 phase.

In various aspects, the polymersomes described herein can be formed using any other methods that are well-known to those of skill in the art, and such methods are incorporated herein by reference. Examples of such polymersomes formation methods are described in Rhim et al., 2016, Macromol. Research, 24:577-586 and Blanazs et al., 2009, Marcomol. Rapid Commun., 30:267-277.

### Method of Imaging

The present disclosure also relates to methods using the nanoparticles or compositions thereof of the present invention. In one aspect, the present disclosure relates to the nanoparticle composition and methods for selectively imaging and providing therapy to biological tissue. In one embodiment, the method of imaging comprises: contacting a biological material with at least one nanoparticle or compositions thereof of the present invention; exposing the biological material to irradiation at a wavelength between 650 and 1400 nm; transducing the resulting ultrasound signal from the biological material; and producing an image in a data processor from the transduced ultrasound signal. In one embodiment, the biological material is a biological tissue. In one embodiment, the biological tissue is a cancer cell. In one embodiment, the cancer cell is present in a mammal.

In one embodiment, the method of imaging comprises: providing at least one nanoparticle or compositions thereof; applying energy to a biological tissue comprising the nanoparticle composition; wherein applying energy to the biological tissue results in at least partial vaporization of the organic liquid; and imaging a biological tissue comprising the nanoparticle contrast agent composition. In one embodiment, applying energy to the biological tissue comprises irradiating at least a portion of the biological tissue with a light source or applying a radio frequency field.

In various embodiments, the method of imaging a biological tissue comprises application of at least one imaging technique. Examples of imaging techniques include, but are not limited to: photoacoustic imaging, ultrasound imaging, optical imaging, magnetic resonance imaging, computed tomography, thermal imaging, nuclear imaging, magnetomotive imaging enhancement, and any combination thereof.

In one embodiment, the method of imaging comprises the steps of: providing a sample, administering one or more biodegradable nanoparticle compositions to the sample, and imaging the one or more biodegradable nanoparticle compositions in the sample, wherein the biodegradable nanoparticle composition is degraded by the sample after imaging.

In various aspects, the method of imaging comprises the use of the nanoparticles or compositions thereof for ultrasound and photoacoustic imaging as well as many other imaging and therapeutic applications.

In one embodiment, the method of imaging comprises providing a nanoparticle composition comprising: an organic liquid comprising a plurality of nanoparticles dispersed therein, and a coating material disposed around the exterior surface of the organic liquid; and imaging a biological tissue comprising the nanoparticle composition.

Some embodiments of the present disclosure provide methods of using nanoparticle compositions to detect the size and proper boundaries of tumor regions. In one embodiment, nanoparticle compositions of the present disclosure may be delivered to cancerous tissue. Delivery methods may include subject injection of nanoparticle compositions, and may also include using targeting domains to help facilitate accumulation in a diseased tissue. Kumar et al. (2008), Korpanty et al. (2005), Byrne et al. (2008). It is believed that this method may provide two or more mechanisms of enhancing diagnostic imaging contrast. When used in conjunction with a combined photoacoustic and ultrasound imaging system, the nanoparticle compositions may strengthen photoacoustic signals from the tumor region while simultaneously increasing ultrasound contrast. When iron oxide nanoparticles are included in the nanoparticle compositions, magnetic resonance imaging, and photoacoustic and/or ultrasound imaging may be used in conjunction. Therefore, two or more imaging modalities may be used by clinicians to verify the location and size of diseased tissue by using a simple injection of nanoparticle compositions.

Additionally, according to embodiments of the present invention, nanoparticle compositions comprising both therapeutic agents and targeting domains may act as a targeted delivery system for therapeutic agents.

Some embodiments provide methods for the use of the organic gas bubbles as vascular blocking agents to initiate necrosis in a specific location of tissue (e.g., blocking tumor vasculature). In some embodiments, deposition of nanoparticle compositions at the region of necrosis would permit photoacoustical monitoring of the decay.

Ultrasound or cMUT transducers are used to detect the mechanically generated acoustic wave signals at the sample surface. The pressure field generated by the laser pulses and subsequently detected after interacting with heterogeneously absorbing and scattering tissue provides information about the spatial distribution of the absorbed electromagnetic energy. This permits mapping of the absorbed energy distribution within the tissue by its acoustic profile. The generation of sound waves by incident radiation is known as the “photoacoustic” or “optoacoustic” effect and is reviewed by Tam (Reviews of Modern Physics, 1986, 58(2), p381-431).

The incident radiation may be any type of energetic radiation, including electromagnetic radiation from radiofrequency to X-ray, electrons, protons, ions, and other particles. For simplicity, all of the above will be referred to herein as “radiation”. The word “light” will be used specifically to denote electromagnetic radiation of any wavelength or frequency. Preferred radiation is in the near IR spectrum, and may be generated by laser, microwave, etc.

Photoacoustic depth profiling can be performed when the measured sound wave is analyzed in terms of transit time from the site of light absorption back to the detector. Signals from deep within a sample take longer to reach the detector than those from regions near the surface. For pulsed irradiation the longer transit time translates into a larger separation between the time of arrival of the pulse and the arrival of the signal at the detector. For amplitude-modulated irradiation, the longer transit time translates into a phase change in the detected sound wave. Together photoacoustic microscopy and photoacoustic depth profiling constitute photoacoustic imaging.

The use of short bursts of light rather than continuously applied light may be helpful for photoacoustic depth profiling. In this case, the absorption of each light pulse and subsequent heating of the various regions of the sample produces one or more positive or negative pressure waves that propagate radially from the site of absorption after each pulse. For very short light pulses, the shape of the pressure pulses generated by the light pulses is determined by the optical and thermal properties, sizes and shapes of the different regions of the sample, as well as by the speed of sound within the sites and the surrounding medium (see for example, Karabutov et al., 1996, Appl. Phys., 63, p545-563; Hutchins, 1986, Can. J. Phys., 64, p1247-1264).

Contrast agents permit light absorption and sound generation in regions not otherwise possible. Contrast agents may also improve signal to noise ratio by increasing the amplitude of the sound wave. Increasing the sound wave amplitude allows an increase in the possible maximum depth of detection and thereby allows imaging of objects further below the surface of the body.

The use of contrast media provides significant amplification of the signal strength, and thus permits improved imaging. Such a contrast agent for photoacoustic imaging works by either (i) enhancing the pre-existing photoacoustic effect or (ii) creating a photoacoustic effect where this was previously not possible. This may be achieved by selectively absorbing radiation in certain organs or healthy or diseased bodily structures or parts thereof, and/or by efficiently converting the radiation into heat, and/or by facilitating or improving heat-pressure conversion, and/or by scattering and diffusing the incident light so that it more uniformly illuminates the target organs.

Tissue of particular interest for imaging include, without limitation, tissues not shielded by bone, e.g., breast tissue, liver tissue: etc.; and blood vessels, which have been found to provide for unexpected amplification of signal. Subjects of interest for imaging include those suspected or know to have liver cancer, breast cancer, atherosclerosis, soft tissue sarcomas, and the like.

The preferred dosage of the contrast media will vary according to a number of factors, such as the administration route, the age, weight and species of the subject, but in general containing in the order of from 1 μmol/kg to 1 mmol/kg bodyweight of the contrast agent.

Administration may be parenteral (e.g., intravenously, intraarterially, intramuscularly, interstitially, subcutaneously, transdermally, or intrasternally) or into an externally voiding body cavity (e.g., the gastrointestinal tract, bladder, uterus, vagina, nose, ears or lungs), in an animate human or non-human (e.g., mammalian, reptilian or avian) body. Usually administration is accomplished by intravenous or intratumor injection.

Imaging of the desired area is performed by detection and appropriate analysis of the sound waves resulting from irradiation. Detection may be performed at the same surface of the sample as the source of incident radiation (reflection) or alternatively at another surface such as the surface diametrically opposed to the incident light, i.e., the sample's back surface (transmission). Suitable methods of detection include the use of a microphone, piezoelectric transducer, capacitance transducer, fiber-optic sensor, cMUT, or alternatively non-contact methods (see Tam, 1986, supra for a review). Techniques and equipment used in ultrasound imaging may be used.

The methods and uses described herein are especially useful for imaging blood-containing structures (e.g., blood vessels), which may be present in tumors, diseased tissue or particular organs, by the use of contrast agents with specificity for that region/structure, e.g., by use of biological recognition agents with the desired specificity.

Continuous wave radiation may be used with its amplitude or frequency modulated. When continuous wave radiation is used, the photoacoustic effects may be analyzed in the frequency domain by measuring amplitude and phase of one or several Fourier components. Alternatively, and preferably, short pulses (impulses) of radiation are employed which allow stress confinement. When pulses are used, analysis may be made in the time domain, i.e., on the basis of the time taken for the sound wave to reach the detector, thus simplifying analysis and aiding depth profiling.

Information obtained from the methods of the invention described herein can be used alone, or in combination with other information (e.g., age, family history, disease status, disease history, vital signs, blood chemistry, PSA level, Gleason score, lymph node staging, metastasis staging, expression of other gene signatures relevant to outcomes of a disease or disorder, such as cancer, etc.) from the subject or from the biological sample obtained from the subject. In some embodiments, the imaging data is combined or correlated with other data or test results that include, but are not limited to measurements or results from serologic testing methods, enzyme immunoassay (EIA), complement fixation (CF), immunodiffusion, clinical presentation, serology, radiography, histology, culture, and clinical parameters or other algorithms for developing or having a disease or disorder, such as cancer. In one embodiment, data include, but are not limited to age, ethnicity, PSA level, Gleason score, lymph node staging, metastasis staging, and other genomic data, and specific expression values of other gene signatures relevant to infection outcomes. In one embodiment, the data comprises subject information, such as medical history, travel history, and/or any relevant family history. Several serology techniques that can be used in combination with the compositions and methods of the present invention. Examples of serology techniques include, but are not limited to: ELISA, agglutination, precipitation, complement-fixation, fluorescent antibodies, and chemiluminescence.

In various embodiments, the subject is a human subject, and may be of any race, ethnicity, sex, and age.

In some embodiments, the size (e.g., average diameter of the nanoparticle) of the nanoparticle or compositions thereof allows for passive diffusion into tumor tissues, and, therefore, may be easily used to image many pathologies. In some embodiments, where the of the nanoparticle or compositions thereof is on a smaller scale, the small size (e.g., average diameter of the nanoparticle) allows the of the nanoparticle or compositions thereof to travel almost anywhere in the body where imaging and/or therapy may need to be performed. For example, in some embodiments, the method comprises nanoparticles and therapeutic agents that act as an optically triggered therapeutic agent delivery and therapeutic agent release systems.

### Method of Treatment and Delivery of Therapeutic Agent

The nanoparticles of the present invention can also be used not only for detecting and imaging, but also for treating a disease or disorder and/or for delivering the nanoparticles or compositions thereof of the present invention to a subject. Thus, in various aspects, spectrally-tunable optical nanoparticles permit not only the imaging and identification of a disease or disorder as a marker of cells associated with the disease or disorder, but may also be used to treat the disease or disorder as well at the time of identification.

In various aspects, the present invention provides a method of delivering the nanoparticles or compositions thereof of the present invention to a subject. In one aspect, the present invention provides a method of delivering the nanoparticles or compositions thereof of the present invention to a biological tissue. In various embodiments, the method of delivering the nanoparticles or compositions thereof comprises a nanoparticle that is stable in the subject. In some embodiments; the method of delivering the nanoparticles or compositions thereof comprises a nanoparticle that is stable in the biological tissue.

In various embodiments, the nanoparticle is stable for at least about 1 hour to at least about 30 hours. In one embodiment, the nanoparticle is stable for at least about 1 hour. In one embodiment, the nanoparticle is stable for at least about 2 hours. In one embodiment, the nanoparticle is stable for at least about 3 hours. In one embodiment, the nanoparticle is stable for at least about 4 hours. In one embodiment, the nanoparticle is stable for at least about 5 hours. In one embodiment, the nanoparticle is stable for at least about 6 hours. In one embodiment, the nanoparticle is stable for at least about 7 hours. In one embodiment, the nanoparticle is stable for at least about 8 hours. In one embodiment, the nanoparticle is stable for at least about 9 hours.

In one embodiment, the nanoparticle is stable for at least about 10 hours. In one embodiment, the nanoparticle is stable for at least about 11 hours. In one embodiment, the nanoparticle is stable for at least about 12 hours. In one embodiment, the nanoparticle is stable for at least about 13 hours. In one embodiment, the nanoparticle is stable for at least about 14 hours. In one embodiment, the nanoparticle is stable for at least about 15 hours. In one embodiment, the nanoparticle is stable for at least about 16 hours. In one embodiment, the nanoparticle is stable for at least about 17 hours. In one embodiment, the nanoparticle is stable for at least about 18 hours. In one embodiment, the nanoparticle is stable for at least about 19 hours. In one embodiment, the nanoparticle is stable for at least about 20 hours. In one embodiment, the nanoparticle is stable for at least about 21 hours. In one embodiment, the nanoparticle is stable for at least about 22 hours. In one embodiment, the nanoparticle is stable for at least about 23 hours. In one embodiment, the nanoparticle is stable for at least about 24 hours. In one embodiment, the nanoparticle is stable for at least about 25 hours. In one embodiment, the nanoparticle is stable for at least about 26 hours. In one embodiment, the nanoparticle is stable for at least about 27 hours. In one embodiment, the nanoparticle is stable for at least about 28 hours. In one embodiment, the nanoparticle is stable for at least about 29 hours. In one embodiment, the nanoparticle is stable for at least about 30 hours.

For example, in some embodiments, the method of delivering the nanoparticles or compositions thereof comprises a nanoparticle, comprising at least one ICGJ that is stable in the subject. In some embodiments, the method of delivering the nanoparticles or compositions thereof comprises a nanoparticle, comprising at least one ICGJ that is stable in the subject and does not dissociate into ICG for at least about 1 hour to at least about 30 hours. In one embodiment, the method of delivering the nanoparticles or compositions thereof comprises a nanoparticle, comprising at least one ICGJ that is stable in the subject and does not dissociate into ICG for at least about 30 hours.

In some embodiments, the method of delivering the nanoparticles or compositions thereof comprises a nanoparticle, comprising at least one ICGJ that is stable in the biological tissue. In some embodiments, the method of delivering the nanoparticles or compositions thereof comprises a nanoparticle, comprising at least one ICGJ that is stable in the biological tissue and does not dissociate into ICG for at least about 1 hour to at least about 30 hours. In one embodiment, the method of delivering the nanoparticles or compositions thereof comprises a nanoparticle, comprising at least one ICGJ that is stable in the biological tissue and does not dissociate into ICG for at least about 30 hours.

In various aspects, the present invention provides a method of treating a disease or disorder in a subject comprising the steps of: administering one or more nanoparticle compositions described herein to the sample; facilitating release of a therapeutic agent in the body from the composition upon degradation or swelling either with or without exposure to laser, high-intensity non-coherent electromagnetic irradiation, RF irradiation, or magnetic field; and destroying cells that contribute to the disease or disorder by photothermolysis of the cells.

In one aspect, the method of treating a disease or disorder comprises: contacting a biological tissue with a contrast agent composition comprising an organic liquid comprising a plurality of nanoparticles or compositions thereof of the present invention, a coating material disposed around the exterior surface of the organic liquid, and a therapeutic agent; and applying energy to the biological tissue, wherein applying energy to the biological tissue results in at least partial vaporization of the organic liquid. In one embodiment, applying energy to the biological tissue comprises irradiating at least a portion of the biological tissue with a light source or applying a radio frequency field. In one embodiment, applying energy to the biological tissue results in release of the therapeutic agent from the contrast agent composition.

In one embodiment, the method of treating a disease or disorder comprises contacting a biological tissue associated with the disease or disorder with a nanoparticle composition comprising: an organic liquid comprising a plurality of nanoparticles dispersed therein, a coating material disposed around the exterior surface of the organic liquid, and a therapeutic agent.

In some embodiments, the method of treating a disease or disorder comprises extending the intensity of laser exposure that facilitates an additional heating of the nanoparticles in order to transition cells associated with the disease or disorder into apoptosis. In various embodiments, method of treating a disease or disorder comprises using the nanoparticles as part of a treatment regimen for the selective elimination of cells associated with the disease or disorder via apoptosis.

In certain embodiments, the method of treating a disease or disorder comprises a “remotely triggered” functionality. In other words, the system may remain inert in the body until specifically triggered. In some embodiments, the nanoparticle is used advantageously in therapeutic applications such as to first target the nanoparticle to a specified location, and then remotely trigger them into an activated state. Sometimes referred to as a “dual targeted delivery system,” this feature may minimize the side effects of systemic therapeutic agents, microwave ablation therapy, vessel occlusion therapy, photothermal therapy, and nuclear medicine. Additionally, embodiments containing magnetic iron oxide and/or cobalt nanoparticles may provide nanoparticle compositions that can be used in microwave ablation therapy and magnetomotive imaging enhancement.

In one embodiment, the present invention provides a method for treating cancer in a subject comprising the steps of: (i) administering one or more nanoparticle compositions to the subject, wherein the biodegradable nanoparticle composition has an absorbance in the visible region, an absorbance in the NIR range between 700 and 950 nm, or both, (ii) monitoring the uptake of the one or more nanoparticles in the one or more tumor cells or circulating tumor cells, (iii) optionally facilitating necrosis and vaporization of the one or more tumor cells or circulating tumor cells by an exposure to laser, high-intensity non-coherent electromagnetic irradiation, RF irradiation, or magnetic field, (iv) transitioning an aggressive tumor phenotype to a more benign tumor and (v) optionally removing the one or more tumor cells or circulating tumor cells by local resection.

In one aspect, the present invention discloses a photo-thermolysis method of treating cancer by induced cell death comprising the steps of, identifying a subject in need for treatment, administering one or more nanoparticle compositions to the subject, monitoring the uptake of the nanoparticle composition, and facilitating induced cell death by an exposure to laser, high-intensity non-coherent electromagnetic irradiation, RF irradiation, or magnetic field.

In some aspects, the present invention discloses a method of preventing a cancer from metastasizing to other locations in the body using the nanoparticle or compositions thereof.

In one aspect, the present invention provides a method comprising administering a composition described herein to a subject having a disease or disorder. For example, in one embodiment, the method comprises administering a composition described herein to a subject having cancer.

The present invention further provides methods relating to the nanoparticles or compositions thereof of the invention that can be used to establish and evaluate treatment plans for a subject with a disease or disorder. In some aspects, the present invention includes methods for assessing the effectiveness of a treatment of a disease or disorder by imaging and detecting cells associated with the disease or disorder in a biological sample obtained from a subject using the methods of imaging of the present invention. In certain embodiments, the biological sample is obtained from a subject having the disease or disorder or being treated for the disease or disorder. In some embodiments, the invention includes methods for assessing the effectiveness of a treatment of cancer by imaging and detecting cancer cells in a biological sample obtained from a subject using the methods of imaging of the present invention.

The following are non-limiting examples of cancers that can be imaged, detected, and/or treated by the disclosed methods and compositions: acute lymphoblastic; acute myeloid leukemia; adrenocortical carcinoma; adrenocortical carcinoma, childhood; appendix cancer; basal cell carcinoma; bile duct cancer, extrahepatic; bladder cancer; bone cancer; osteosarcoma and malignant fibrous histiocytoma; liposarcoma and anaplastic liposarcoma; brain stem glioma, childhood; brain tumor, adult; brain tumor, brain stem glioma, childhood; brain tumor, central nervous system atypical teratoid/rhabdoid tumor, childhood; central nervous system embryonal tumors; cerebellar astrocytoma; cerebral astrocytotna/malignant glioma; craniopharyngioma; ependymoblastoma; ependymoma; medulloblastoma; medulloepithelioma; pineal parenchymal tumors of intermediate differentiation; supratentorial primitive neuroectodermal tumors and pineoblastoma; visual pathway and hypothalamic glioma; brain and spinal cord tumors; breast cancer; bronchial tumors; Burkitt lymphoma; carcinoid tumor; carcinoid tumor, gastrointestinal; central nervous system atypical teratoid/rhabdoid tumor; central nervous system embryonal tumors; central nervous system lymphoma; cerebellar astrocytoma cerebral astrocytoma/malignant glioma, childhood; cervical cancer; chordoma, childhood; chronic lymphocytic leukemia; chronic myelogenous leukemia; chronic myeloproliferative disorders; colon cancer; colorectal cancer; craniopharyngioma; cutaneous T-cell lymphoma; esophageal cancer; Ewing family of tumors; extragonadal germ cell tumor; extrahepatic bile duct cancer; eye cancer, intraocular melanoma; eye cancer, retinoblastoma; biliary track cancer, cholangiocarcinoma, anal cancer, neuroendocrine tumors, small bowel cancer, gallbladder cancer; gastric (stomach) cancer; gastrointestinal carcinoid tumor; gastrointestinal stromal tumor (gist); germ cell tumor, extracranial; germ cell tumor, extragonadal; germ cell tumor, ovarian; gestational trophoblastic tumor; glioma; glioma, childhood brain stem; glioma, childhood cerebral astrocytoma; glioma, childhood visual pathway and hypothalamic; hairy cell leukemia; head and neck cancer; hepatocellular (liver) cancer; histiocytosis, langerhans cell; Hodgkin lymphoma; hypopharyngeal cancer; hypothalamic and visual pathway glioma; intraocular melanoma; islet cell tumors; kidney (renal cell) cancer; Langerhans cell histiocytosis; laryngeal cancer; leukemia, acute lymphoblastic; leukemia, acute myeloid; leukemia, chronic lymphocytic; leukemia, chronic myelogenous; leukemia, hairy cell; lip and oral cavity cancer; liver cancer; lung cancer, non-small cell; lung cancer, small cell; lymphoma, aids-related; lymphoma, burkitt; lymphoma, cutaneous T-cell; lymphoma, non-Hodgkin lymphoma; lymphoma, primary central nervous system; macroglobulinemia, Waldenstrom; malignant fibrous histiocvtoma of bone and osteosarcoma; medulloblastoma; melanoma; melanoma, intraocular (eye); Merkel cell carcinoma; mesothelioma; metastatic squamous neck cancer with occult primary; mouth cancer; multiple endocrine neoplasia syndrome, (childhood); multiple myeloma/plasma cell neoplasm; mycosis; fungoides; myelodysplastic syndromes; myelodysplastic/myeloproliferative diseases; myelogenous leukemia, chronic; myeloid leukemia, adult acute; myeloid leukemia, childhood acute; myeloma, multiple; myeloproliferative disorders, chronic; nasal cavity and paranasal sinus cancer; nasopharyngeal cancer; neuroblastoma; non-small cell lung cancer; oral cancer; oral cavity cancer; oropharyngeal cancer; osteosarcoma and malignant fibrous histiocytoma of bone; ovarian cancer; ovarian epithelial cancer; ovarian germ cell tumor; ovarian low malignant potential tumor; pancreatic cancer, islet cell tumors; papillomatosis; parathyroid cancer; penile cancer; pharyngeal cancer; pheochromocytoma; pineal parenchymal tumors of intermediate differentiation; pineoblastoma and supratentorial primitive neuroectodermal tumors; pituitary tumor; plasma celt neoplasm/multiple myeloma; pleuropulmonary blastoma; primary central nervous system lymphoma; prostate cancer; rectal cancer; renal cell (kidney) cancer; renal pelvis and ureter, transitional cell cancer; respiratory tract carcinoma involving the nut gene on chromosome 15; retinoblastoma; rhabdomyosarcoma; salivary gland cancer; sarcoma, ewing family of tumors; sarcoma, Kaposi; sarcoma, soft tissue; sarcoma, uterine; Sezary syndrome; skin cancer (nonmelanoma); skin cancer (melanoma); skin carcinoma, Merkel cell; small cell lung cancer; small intestine cancer; soft tissue sarcoma; squamous cell carcinoma, squamous neck cancer with occult primary, metastatic; stomach (gastric) cancer; supratentorial primitive neuroectodermal tumors; T-cell lymphoma, cutaneous; testicular cancer; throat cancer; thymoma and thymic carcinoma; thyroid cancer; transitional cell cancer of the renal pelvis and ureter; trophoblastic tumor, gestational; urethral cancer; uterine cancer, endometrial; uterine sarcoma; vaginal cancer; vulvar cancer; Waldenstrom macroglobulinemia; and Wilms tumor.

In some embodiments, the present invention provides methods of providing a diagnosis or prognosis of a disease or disorder to a subject using the compositions of the present invention. In one embodiment, provides an assessment of the effectiveness of a treatment of a disease or disorder. In one embodiment, the method comprises a) providing a biological sample from the subject; b) analyzing the biological sample with at least one contrast agent of the invention that specifically detects at least one cell associated with the disease or disorder in the biological sample to form at least one contrast agent labelled cell associated with the disease or disorder; c) comparing the amount of contrast agent labelled cells associated with the disease or disorder in the sample with the amount in a comparator sample, wherein a statistically significant difference between the amount of contrast agent labelled cells associated with the disease or disorder in the sample with the amount in a comparator sample or earlier obtained biological sample is indicative of a disease or disorder in the subject. In some embodiments, the method further comprises the step of d) effectuating a treatment regimen based thereon. In one embodiment, the method comprises a) administering to a subject at least one contrast agent of the invention that specifically detects at least one cell associated with the disease or disorder in the biological sample to form at least one contrast agent labelled cell associated with the disease or disorder; b) comparing the amount of contrast agent labelled cells associated with the disease or disorder in the subject or in a specific region of the subject with the amount in a comparator sample, wherein a statistically significant difference between the amount of contrast agent labelled cells associated with the disease or disorder in the sample with the amount in a comparator sample or earlier obtained biological sample is indicative of a disease or disorder in the subject. In some embodiments, the method further comprises the step of c) effectuating a treatment regimen based thereon.

In some embodiments, the method of treatment comprises: imaging and detecting cells associated with a disease or disorder that indicate a treatment of the subject is needed. In one embodiment, the treatment is determined based on the amount of cells associated with the disease or disorder in a subject. In some embodiments, the method of treatment includes, but is not limited to pharmacotherapy, surgery, radiation, and chemotherapy. In some embodiments, the method of treatment comprises administering a therapeutically effective amount of a therapeutic agent. Examples of such therapeutic agents include, but are not limited to: a nucleic acid, a peptide, a small molecule chemical compound, an siRNA, a ribozyme, an antisense nucleic acid, an aptamer, a peptidomimetic, an antibody, an antibody fragment, an antibiotic, antifungal medication, and any combination thereof.

In some embodiments, the method of treatment comprises an assessment of the effectiveness of the treatment regimen for a disease or disorder by detecting the amount of cells associated with the disease or disorder in samples obtained from a subject over time and comparing the detected amounts of cells associated with the disease or disorder. In some embodiments, a first sample is obtained prior to the subject receiving treatment and one or more subsequent samples are taken after or during treatment of the subject. In some embodiments, changes in the amounts of cells associated with the disease or disorder over time provide an indication of effectiveness of the therapy.

In some embodiments, the methods of the present invention comprise effecting a therapy and/or the treatment regime based on the diagnosis or assessment of prognosis of a disease or disorder. In one embodiment, the treatment is adjusted based on the amount of cells associated with a disease or disorder in a subject.

In one aspect, the amount of cells associated with cancer are used to monitor subjects undergoing treatments and therapies for a cancer, subjects who have had a cancer, and subjects who are in remission of a previously diagnosed and treated cancer. In one embodiment, the amount of cells associated with a cancer are used to select or modify treatments in subjects having a cancer, subjects who have had a cancer, and subjects who are in remission of a previously diagnosed and treated cancer.

In one aspect, the present invention also provides methods for identifying agents for treating a disease or disorder that are appropriate or otherwise customized for a specific subject. In one embodiment, a test sample from a subject, exposed to a therapeutic agent or a drug, can be taken and the amount of cells associated with a disease or disorder can be determined. In one embodiment, the amount of cells associated with a disease or disorder can be compared to a sample derived from the subject before and after treatment, or can be compared to samples derived from one or more subjects who have shown improvements or alleviation of a disease or disorder as a result of such treatment or exposure.

To identify therapeutic agents that are appropriate for a specific subject, a test sample from the subject can also be exposed to a therapeutic agent or a drug, and the amount of cells associated with a disease or disorder can be determined. The amount of cells associated with a disease or disorder can be compared to a sample derived from the subject before and after treatment or exposure to a therapeutic agent or a drug, or can be compared to samples derived from one or more subjects who have shown improvements relative to a disease as a result of such treatment or exposure. Thus, in one aspect, the invention provides a method of assessing the efficacy of a therapy with respect to a subject comprising a step of taking a first measurement of the amount of cells associated with a disease or disorder in a first sample from the subject; a step of effecting the therapy with respect to the subject; a step of taking a second measurement of the amount of cells associated with a disease or disorder in a second sample from the subject; and a step of comparing the first and second measurements to assess the efficacy of the therapy.

In various exemplary embodiments, the methods of the invention include effecting a therapy for the treatment of a diagnosed disease. In one embodiment, effecting a therapy comprises administering a disease-modulating therapeutic agent to the subject. In various embodiments, effecting a therapy comprises treatment of one or more symptoms of the disease or disorder. For example, in one embodiment, effecting a therapy comprises administration of a non-disease-modulating drug to the subject. Exemplary non-disease-modulating drugs that may be administered include, but are not limited to, pain relievers, anti-inflammatory drugs, NSAIDs, decongestants, cough suppressants, including topical cough suppressants, or other agents that may function to reduce the severity of at least one symptom of the disease or disorder.

Any therapeutic agent or any combination of therapeutic agents disclosed herein may be administered to a subject to treat a disease or disorder. The therapeutic agents herein can be formulated in any number of ways, often according to various known formulations in the art or as disclosed or referenced herein.

In various embodiments, one or more additional drugs may be optionally administered in addition to those that are recommended or have been administered. An additional drug will typically not be any drug that is not recommended or that should be avoided.

In some embodiments, the nanoparticles or compositions thereof undergo uptake into biological sample. In some embodiments, the nanoparticles or compositions thereof undergo uptake into macrophage cells. In some embodiments, the nanoparticles or compositions thereof undergo uptake into macrophage cells associated with tumors, atherosclerosis, and arthritis. For example, in some embodiments, the nanoparticles or compositions thereof can be coated with dextran to target the macrophage cells, since macrophages have dextran receptors. In various embodiments, the method further comprises allowing the nanoparticle or compositions thereof to accumulate in a region of the biological tissue, wherein the targeting domain facilitated accumulation of the nanoparticle or compositions thereof in the region.

In various aspects, the nanoparticle or compositions thereof of the present invention can be used alone or in combination with a therapeutic agent to deliver a therapeutic agent payload to a target cell. Often, the therapeutic agent may be released based on the degradation of, e.g., a controlled release biodegradable matrix and/or polymer. However, it has been found that the nanoparticles of the present invention can also deliver their payload by laser heating or optical disruption of the nanoparticles.

In one aspect, the present invention discloses a method by which a therapeutic agent can be delivered to a subject in need of a therapeutic agent. In some embodiments, the biodegradable nanoparticle composition of the present invention that are administered to the subject comprises one or more therapeutic agents. In various embodiments, the therapeutic agent is released upon biodegradation of the nanoparticles or by irradiating or heating the nanoparticles with a laser in a NIR region.

Thus, in various aspects, the present invention also provides a method for delivering a therapeutic agent comprising the steps of: identifying a subject in need of the therapeutic agent; administering the therapeutic agent; wherein the therapeutic agent is associated with a biodegradable nanoparticle comprising one or more nanoparticles and one or more stabilizers; and releasing the therapeutic agent by heating the particles with a laser or other optical source in a NIR region. In certain aspects, the biodegradable nanoparticle comprises a hydrodynamic diameter smaller than 100 nm and has an absorbance in the NIR window between 700 nm and 1100 nm.

The nanoparticles or compositions thereof of the present invention can be adapted for administration using a wide variety of methods of delivery, including, but not limited to, e.g., subcutaneous, intravenous, peritoneally, orally, intramuscular, topical, nasally, intradermal, ocular, rectal, vaginal and combinations thereof.

### Kits

The present invention also pertains to kits useful in the methods of the invention. Such kits comprise various combinations of components useful in any of the methods described elsewhere herein, including for example, materials for preparing the nanoparticles of the invention, materials for imaging techniques using the nanoparticles of the invention, materials for detecting a disease or disorder using the nanoparticles of the invention, materials for treating a disease or disorder using the nanoparticles of the invention, and instructional material. For example, in one embodiment, the kit comprises components useful for the preparation of the desired nanoparticle. In a further embodiment, the kit comprises components useful for the imaging techniques of a desired cells associated with a disease or disorder in a biological sample. In a further embodiment, the kit comprises components useful for the detection of a desired cells associated with a disease or disorder in a biological sample. In a further embodiment, the kit comprises components useful for the treatment of a desired disease or disorder in a subject.

In a further embodiment, the kit comprises the components of an assay for monitoring the effectiveness of a treatment administered to a subject in need thereof, containing instructional material and the components for determining whether the level of cells associated with a disease or disorder, detected using the nanoparticles of the invention, in a biological sample obtained from the subject is modulated during or after administration of the treatment. In various embodiments, to determine whether the level of cells associated with a disease or disorder, detected using the nanoparticles of the invention, is modulated in a biological sample obtained from the subject, the level of cells associated with a disease or disorder is compared with the level of at least one comparator contained in the kit, such as a positive control, a negative control, a historical control, a historical norm, or the level of another reference molecule in the biological sample. In certain embodiments, the ratio of the cells associated with a disease or disorder, detected using the nanoparticles of the invention, and a reference molecule is determined to aid in the monitoring of the treatment.

### EXPERIMENTAL EXAMPLES

The invention is further described in detail by reference to the following experimental examples. These examples are provided for purposes of illustration only, and are not intended to be limiting unless otherwise specified. Thus, the invention should in no way be construed as being limited to the following examples, but rather, should be construed to encompass any and all variations which become evident as a result of the teaching provided herein.

Without further description, it is believed that one of ordinary skill in the art can, using the preceding description and the following illustrative examples, make and utilize the present invention and practice the claimed methods. The following working examples therefore, specifically point out the preferred embodiments of the present invention, and are not to be construed as limiting in any way the remainder of the disclosure.

### Example 1: Indocyanine Green J Aggregates in Polymersomes for Near IR Photoacoustic Imaging

The data presented herein demonstrates encapsulation of ICG J aggregates with very limited dissociation into PLGA-b-PEG (90K:10K) polymersomes (Ps) with grafted monoclonal antibodies (mAbs) to target cancer cells for high contrast NIR PAI.

All materials that were used in Example 1 were FDA approved materials in order to facilitate future clinical translation. This data demonstrates that ICGJ may dissociate as highly surface active ICG molecules migrate from the ICGJ surface and adsorb at the oil-water interface during emulsion formation. Thus, this driving force for dissociation was lowered by providing excess ICG to adsorb at the interface (“sacrificial ICG”) and with the electrostatic assembly of a thin protective cationic shell of branched polyethyleneimine (PEI) onto the anionic ICG J aggregate surface (ICGJ@PEI). With these approaches, ICGJ@PEI survive the various interfaces encountered in a W/O/W emulsion/evaporation process and subsequent Ps purification. As the oil droplets shrink during evaporation, and as a consequence of the interfacial properties (Hayward R C et al., 2006, Langmuir, 22:4457-4461), relatively monodisperse ˜77 nm Ps are released into water. The hydrodynamic diameter and the NIR spectra of the ICGJ@PEI loaded Ps were shown to be stable for at least 28 days in DI water indicating an inert PEG-PLGA bilayer with respect to ICGJ@PEI. The stabilization mechanisms during Ps formation and storage are discussed in terms of the properties of the PEI shell and adsorption of ICG and PLGA-b-PEG at various interfaces, along with the colloidal interactions. Strong NIR extinction of the encapsulated ICG dye J aggregates was achieved at 895 nm in 100% FBS at 37° C. over 24 hours as desirable for PAI in vivo, and after one week in 100% FBS, the biodegradable nanocapsules fully dissociated and released ICG. Ps-encapsulated ICG@PEI was conjugated with antibodies specific for epidermal-growth factor receptor (EGFR), which is one of major cancer hallmarks (Hanahan D et al., 2011, Cell, 144:646-674). Breast and ovarian cancer cells labeled with antibody-conjugated Ps were detected by PAI with high specificity and at concentrations as low as a few cells per mm 3 in tissue mimicking phantoms.

### Instability of ICGJ at the Water-Chloroform Interface

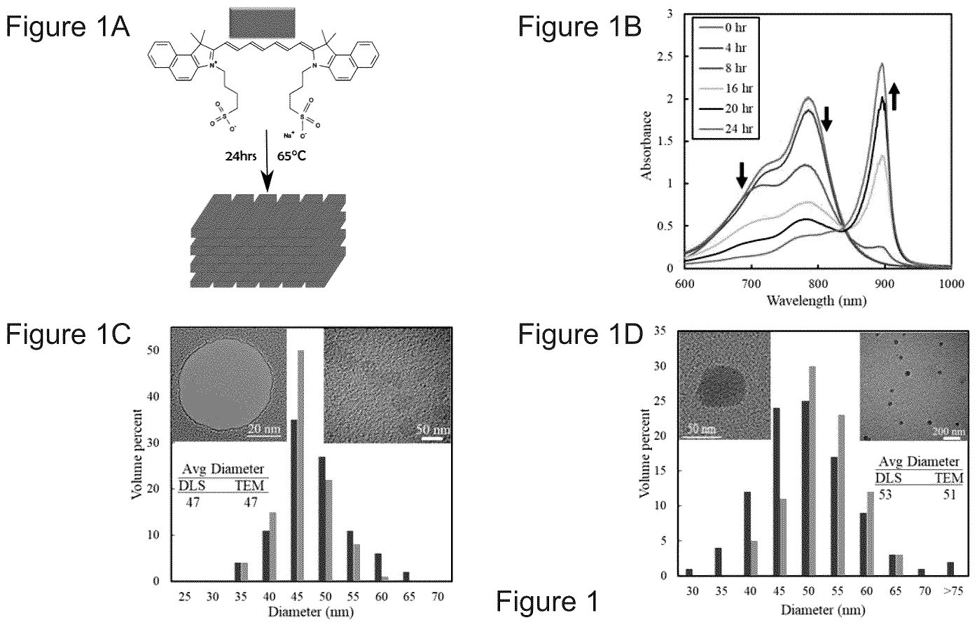

The experiments described herein were conducted to characterize and understand the dissociation of ICGJ at the water-chloroform interface in order to develop strategies to avoid it during encapsulation into polymersomes. In an aqueous solution, ICG is mainly present in the form of monomers and dimers with two absorption peaks at 780 nm and 715 nm, respectively. Upon incubation at 65° C. for 24 hours, non-covalent π-π stacking and hydrophobic interactions promote self-association into ICGJ (FIG. 1A) characterized by a sharp extinction peak at 895 nm (FIG. 1B) with a J aggregate to monomer absorbance ratio characterized by an I₈₉₅/I₇₈₀ of ˜5 (Landsman M et al., 1976, Journal of Applied Physiology, 40:575-583). The volume average diameter of ICGJ particles was ˜50 nm, measured by DLS and TEM (FIG. 1C), comparable to a reported value (Liu R et al., 2017, Nanotheranostics, 1:430-439). The sharp 895 nm peak in the NIR window provides opportunities for deep tissue imaging with reduced photobleaching given limited access to 02 in the hydrophobic J aggregate interior (Sheng Z et al., 2014, ACS Nano, 8:12310-12322; Huang P et al., 2014, Advanced Materials, 26:6401-6408).

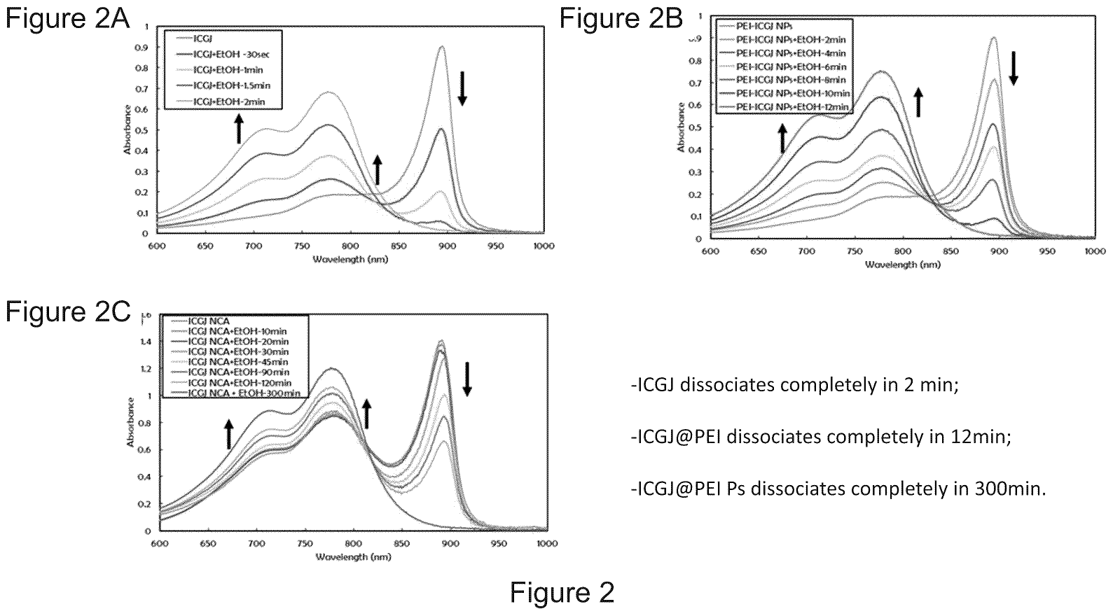

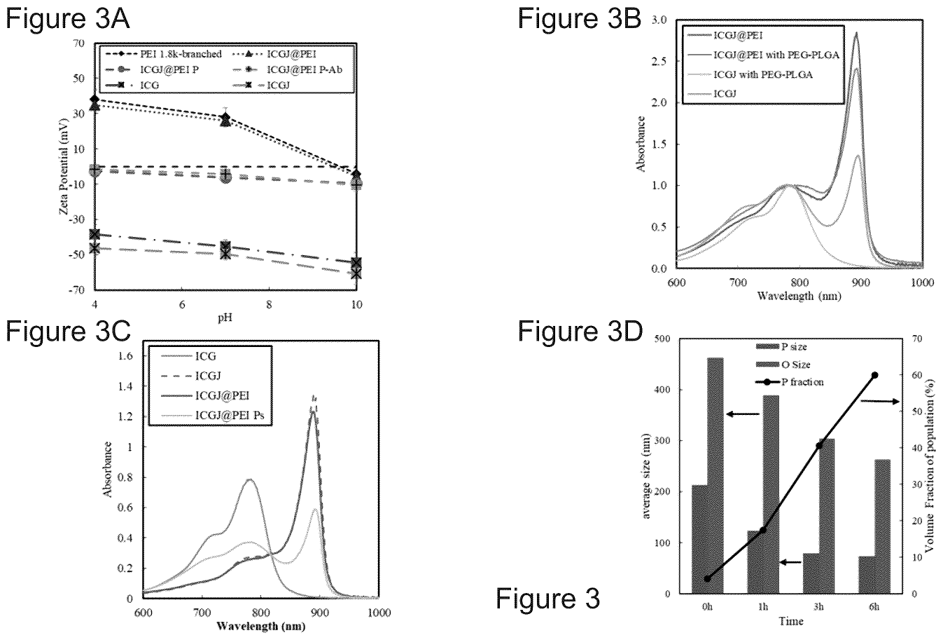

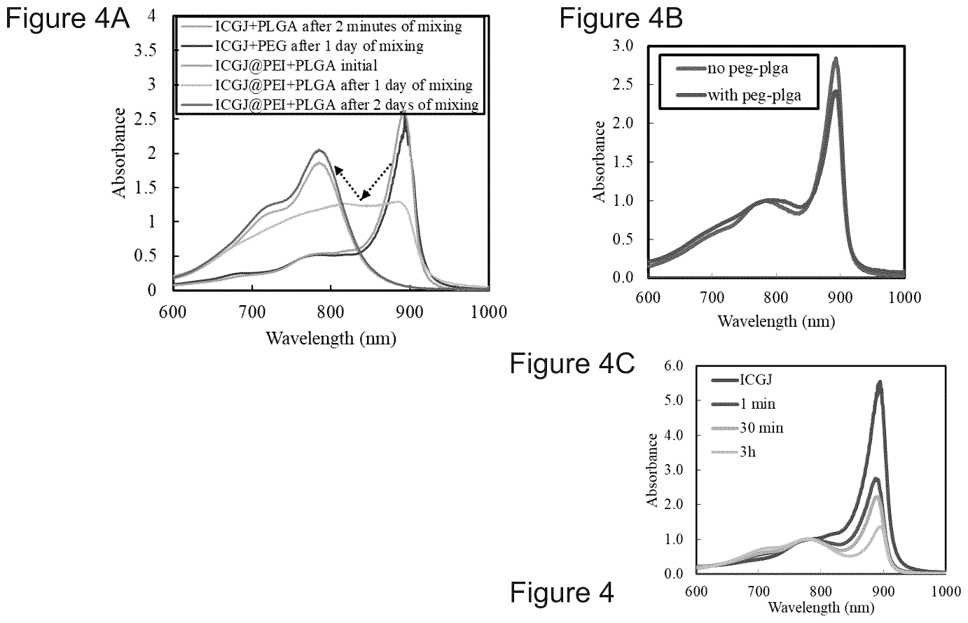

It is known that ICGJ readily dissociates back into ICG monomers upon addition of organic solvents, such as 10% v/v ethanol/water, (FIG. 2) or amphiphilic detergents (ex. Tween 80, Triton x-100) (Liu R et al., 2017, Nanotheranostics, 1:430-439). Additionally, ICGJ may be destabilized by the water-surfactant interface in lipid bilayers, particularly for less rigid bilayers (Desmettre T et al., 2000, Survey of Ophthalmology, 45:15-27; Zheng X et al., 2011, Molecular Pharmaceutics, 8:447-456; Yoneya S et al., 1998, Investigative Ophthalmology & Visual Science, 39:1286-1290; Mordon S et al., 1998, Microvascular Research, 55:146-152). Similarly, in human blood, ICGJ dissociates due to interactions with lipoproteins, free cholesterol, and phospholipids (Yoneya S et al., 1998, Investigative Ophthalmology & Visual Science, 39:1286-1290). The results presented herein also demonstrate that ICGJ dissociates upon exposure to the W/O emulsion interface upon homogenizing 50 mg/mL ICGJ in 0.5 mL of water with 1 mL of chloroform using an ultra-Turrax, as I₈₉₅/I₇₈₀ peak ratio dropped to 1.3 after 3 hours (FIG. 3B) relative to an initial value of ˜5 (FIG. 3C). When the same experiment was conducted by applying a low-shear using a high-speed stirring with a stir bar for 24 hours, an emulsion was not formed, and the I₈₉₅/I₇₈₀ remained stable given the low interfacial surface area. Upon modifying this experiment by adding 25 mg/mL surface active PEG-PLGA, a W/O emulsion was formed with high interfacial area, and consequently, the J aggregates fully dissociated in only 2 minutes (FIG. 3B and FIG. 4). Thus, ICGJ dissociates as ICG molecules leave the J aggregates to adsorb at the water-chloroform interface to lower the interfacial tension.

ICG is highly surface active and adsorbs at a level of 2.6 nm²/molecule at the water/air interface (Ikagawa H et al., 2005, Invest Ophthalmol Vis Sci., 46:2531-2539). At this adsorption level, a theoretical ICG concentration of 60 mg/mL would be needed to cover the interface of ˜50 nm water droplets in chloroform for 0.5 mL of water and 1 mL of chloroform. An assumed length scale of 50 nm water droplets is based on the size of the Ps from DLS and TEM shown below. Thus, an extensive amount of ICGJ dissociation would be expected to take place upon attempting to load Ps with an experimental concentration, such as 50 mg/mL ICGJ. Therefore, novel strategies were needed to protect ICGJ against dissociation driven by interfacial adsorption of ICG during Ps formation.

Formation and Characterization of ICGJ@PEI Nanoparticles with Enhanced Stability at the Water-Chloroform Interface

To help protect ICGJ against dissociation at the water-chloroform interface and ultimately in Ps, a thin layer of branched cationic PEI (Mw: 1800 g/mol) was adsorbed onto the anionic ICGJ surface. As shown in FIG. 1D, the adsorption of PEI only increased the D_{H} by a few nm to 53 nm by DLS. The electrostatic adsorption mechanism of PEI, as a weak base, on an oppositely charged ICGJ surface involves diffusion, surface attachment and surface rearrangement (Geffroy C et al., 2000, Colloids and Surfaces A: Physicochemical and Engineering Aspects, 172:47-56; Claesson P M et al., 2005, Adv. Colloid Interface Sci., 114:173-187). As shown in FIG. 3A, the zeta potentials of the bare ICGJ nanoparticles (NPs) and ICG monomer are highly negative in the range of −46 to −61 mV in the pH range of 4-10 given the negatively charged weakly basic sulfonate groups (Gholibegloo E et al., 2018, Journal of Photochemistry and Photobiology B: Biology, 181:14-22). Note that ICG monomer forms small micelles on the order of 3-4 nm that scatter enough light (Gholibegloo E et al., 2018, Journal of Photochemistry and Photobiology B: Biology, 181:14-22) to measure a ζ. This tendency to form small micelles is consistent with the high activity of ICG at water-oil interfaces noted above (Ikagawa H et al., 2005, Invest. Ophthalmol. Vis. Sci., 46:2531-2539). At pH 4 and low ionic strength, the highly protonated cationic branched PEI chains spread on the negatively-charged ICGJ surface to form a pancake-like structure (Claesson P M et al., 2005, Adv. Colloid Interface Sci., 114:173-187; Schneider M et al., 2003, Macromolecules, 36:9510-9518). Previously, it was found that adsorption of branched PEI on highly charged anionic mica results in a larger positive charge (charge overcompensation) than for a linear analog, poly(vinyl amine), given that it brings a larger number of charges to a given surface area (Claesson P M et al., 2005, Adv. Colloid Interface Sci., 114:173-187).

After purification, 93% of the ICGJ (according to the calibration curve in Figure and 54% of PEI (from TOC measurements described below) were recovered in the ICGJ@PEI NPs, corresponding to a ratio of ˜170 PEI molecules per ICGJ aggregate. From this ratio, the calculated shell thickness of PEI was ˜2.0 nm for ICGJ aggregates that were approximated as effective spheres with a diameter of 50 nm, consistent with the small increase in D_{H} by DLS. From the 2D radius of gyration of PEI_{1.8K}, each PEI_{1.8K} molecule occupies an area of ˜65 nm² such that ˜140 molecules would be required to cover the ICGJ surface. The number of molecules of PEI per ICGJ aggregate in the synthesis was chosen to provide an excess about 20% larger than the required ˜140 molecules for a feed of 100 μL 10 wt % PEI added to 500 μL of 50 mg/mL ICGJ. The formation of approximately a monolayer is supported by a ζ that is only a little below the value observed for pure PEI_{1.8K} (FIG. 3A). This overcompensation of charge for the initial anionic surface is greater for branched PEI than linear PEI as it has more charge per unit area (Claesson P M et al., 2005, Adv Colloid Interface Sci., 114:173-187). As the PEI feed was increased to 200 μL, the only increased from +35 (FIG. 3A and FIG. 6) to 38 mV suggesting close to a monolayer PEI coverage was already present for the aforementioned 100 μL sample. The large positive surface charge will inhibit addition of PEI chains beyond a monolayer. Abdullayev et al. reported similar surface charges upon coating PEI on halloysite nanotubes (Abdullayev E et al., 2008, Mater. Sci. Eng., 99:331-332). Finally, we find that the added PEI shell did not affect the UV-vis-NIR spectrum, as seen for ICGJ compared with ICGJ@PEI NPs in FIG. 3C, indicating stability of the underlying ICG J aggregate.

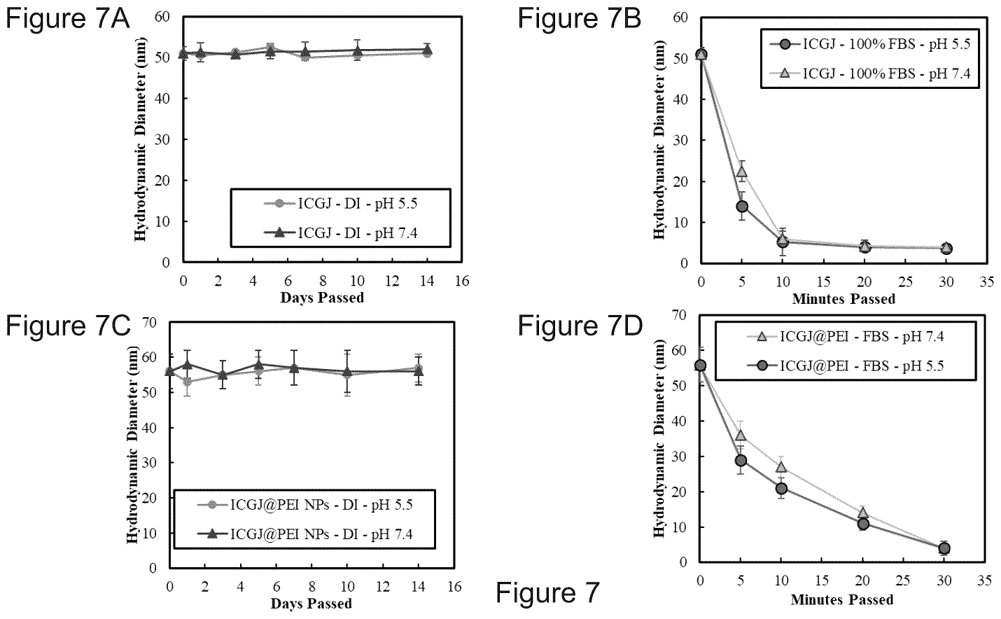

With the protection of the PEI shell in ICGJ@PEI, the I_{895/780} remained 2.8 upon exposure to the water-chloroform interface (without PEG-PLGA) with sonication. In contrast, it dropped to only 1.3 without the PEI coating (FIG. 3B). Similarly, upon vigorous stirring of mixtures of chloroform containing 25 mg PEG-PLGA and ICGJ@PEI in water for one day to form emulsions, the J aggregate dissociation was mitigated (FIG. 4A) relative to the case of uncoated ICGJ aggregates. Thus, a PEI shell was utilized upon encapsulation of ICGJ@PEI into PEG-PLGA Ps to improve stability. The encapsulation with Ps will be important for stabilizing J aggregates for future in vivo applications, given that ICGJ aggregates dissociate quickly in 100% FBS (Shakiba M et al., 2016, Nanoscale, 8:12618-12625) with or without a PEI coating, as shown in FIG. 7.

Formation and Characterization of Stable ICGJ@PEI Encapsulated into Ps with PEG-PLGA Bilayers

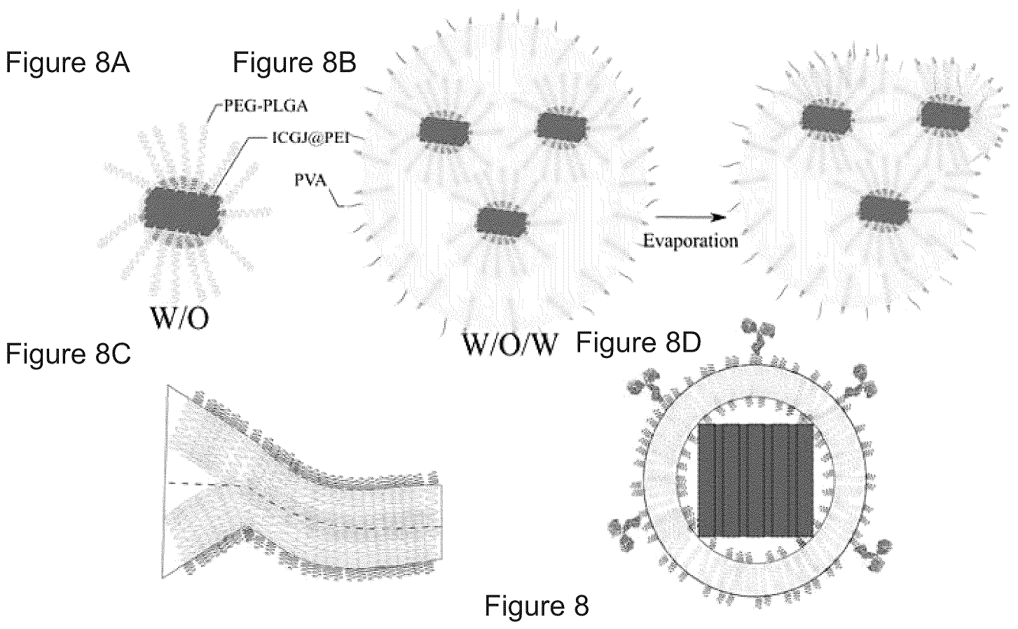

Encapsulation of ICGJ@PEI was performed by a double emulsion solvent evaporation process that may be used to form Ps (FIG. 8) (Blanazs A et al., 2009, Macromol. Rapid Commun., 30:267-277; Hayward R C et al., 2006, Langmuir, 22:4457-4461; Shum H C et al., 2011, Journal of the American Chemical Society, 133:4420-4426). First, a W₁/O emulsion containing water droplets coated with PEG-PLGA was formed by sonication of ICGJ@PEI (W1) in a solution of PEG-PLGA in chloroform (O) (FIG. 8A) (Gu W et al., 2016, Journal of Materials Chemistry B, 4:910-919). This oil continuous phase was then emulsified into a second aqueous phase (W₂) containing polyvinylalcohol (PVA) as a surfactant and steric stabilizer (FIG. 8B). Each oil droplet contained over 200 of the W₁ water droplets based on the volumes of the phases and approximate droplet sizes. As the chloroform evaporates over several hours, it is shown that PEG-PLGA coated W₁ droplets are released from the shrinking oil droplets (Hayward R C et al., 2006, Langmuir, 22:4457-4461). As they leave the oil phase (FIG. 8C), the PEG-PLGA forms a bilayer Ps membrane with the PLGA groups aligned inwards towards each other and PEG extending to the inner and outer water phases (FIG. 8C). A cross section of the final Ps structure is shown in FIG. 8D, after grafting antibodies by click chemistry. The dense PEG brush on the inside of these Ps shells protects the ICGJ@PEI from the destabilizing PLGA groups. Moreover, the dense PEG brush on the outside of these Ps shells contributes to steric stabilization, provides significant protection against protein opsonization, and minimizes the interaction of the hydrophobic PLGA blocks with the physiological environment. For each of these factors, the relatively high PEG molecular weight has the potential to be beneficial compared with Ps typically made with lower molecular weight block copolymers.

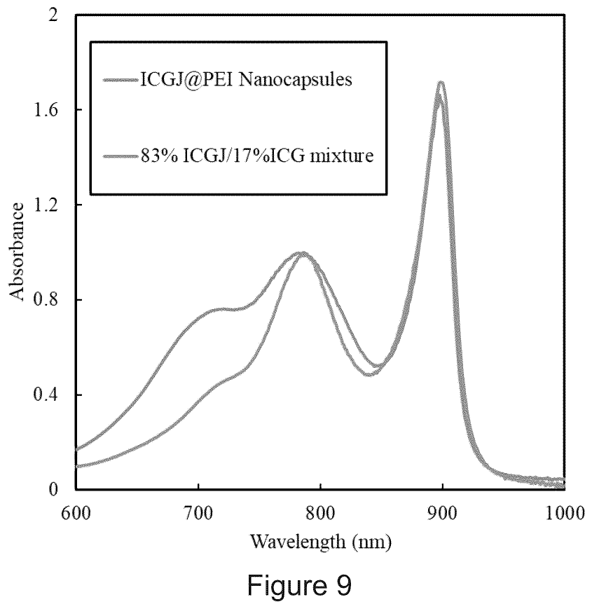

Above branched PEI was shown to partially mitigate the destabilizing effects of the water-chloroform interface during vigorous stirring and even sonication for the inner W₁-O emulsion at pH 4 (FIG. 3B and FIG. 4) where it was positively charged. In a control experiment, the I₈₉₅/I₇₈₀ peak ratio was found to decrease from ˜5, for ICGJ@PEI, to ˜2.8 after exposure to the water-chloroform interface in FIG. 3B (that would be encountered in the first W₁/O emulsion), and finally to ˜1.6 after encapsulation in Ps and final purification (FIG. 3C). For Ps after purification the absorbance at the ICG monomer peak (780 nm) indicates that only about 17% (w/w) of the total amount of the ICG dye molecules are in a monomer state inside the Ps, based on comparison of a mixture of ICG and ICGJ@PEI with similar spectra (FIG. 9). The spectra are shown below to be very suitable for sensitive PAI with near-infrared (NIR) excitation.

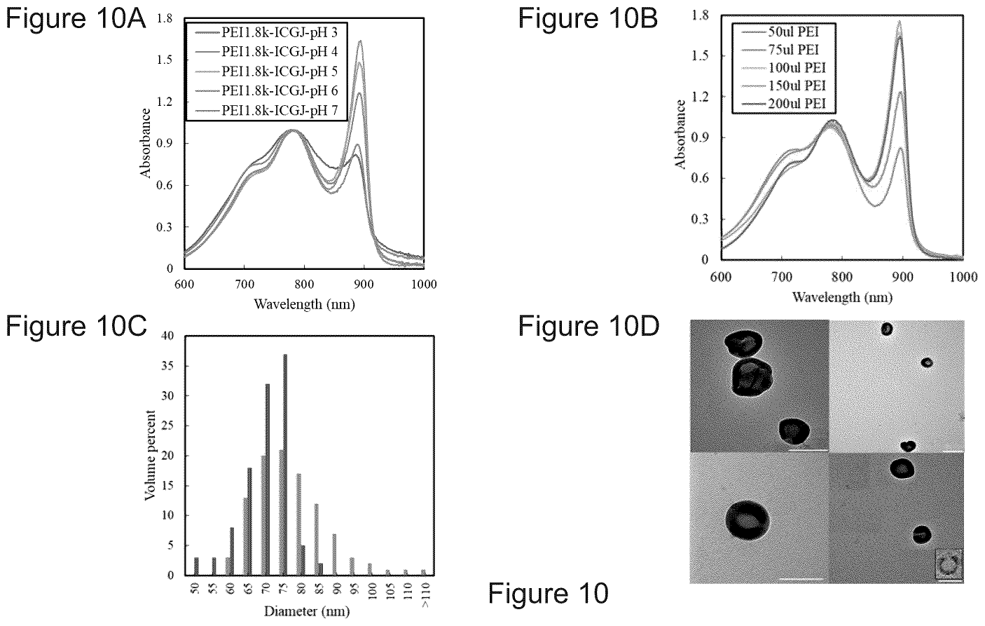

A number of properties and compositions were investigated to develop further mechanistic insight for guiding the formation of Ps containing ICGJ@PEI and to maximize I₈₉₅/I₇₈₀ ratio. ICGJ@PEI formed at various pH values and PEI concentrations was encapsulated into Ps at the standard conditions (see experimental section). I₈₉₅/I₇₈₀ ratio increased as the pH was lowered to 4 (FIG. 10A) as the ICGJ@PEI became highly cationic (FIG. 3A). Likewise, I₈₉₅/I₇₈₀ ratio increased with the amount of PEI used to form ICGJ@PEI at pH 4 and reached a plateau at about 100 μL (FIG. 10B), where also reached a plateau (FIG. 6). In both cases the greater degree electrostatic binding of PEI to the anionic sulfonate groups on ICGJ aided stabilization at the water-chloroform interface during Ps formation.

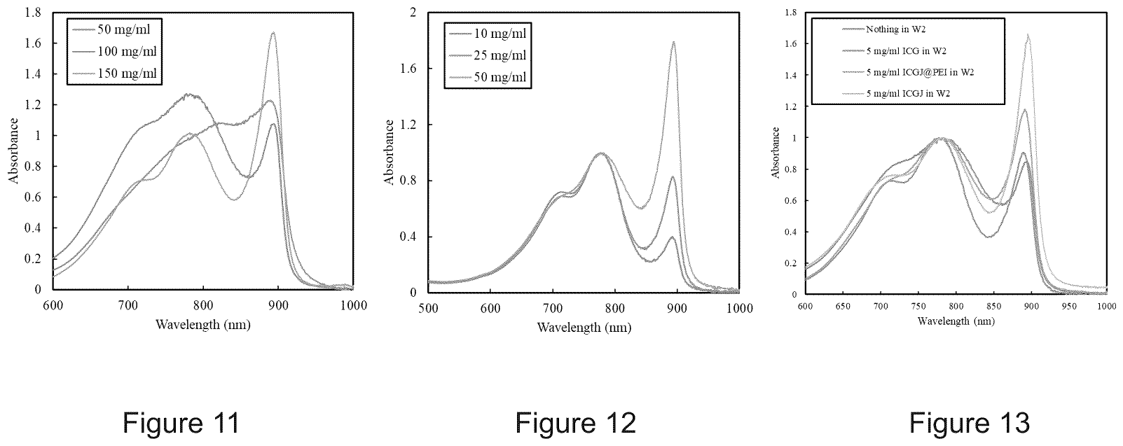

It was also demonstrated that the concentration of ICGJ@PEI in the inner W1 phase or the presence of either ICGJ or ICGJ@PEI in the outer W₂ phase had a large effect on mitigating dissociation of J aggregates during encapsulation. With 50 mg/mL ICGJ@PEI in the W₁ phase, the I₈₉₅/I₇₈₀ was only 0.8 after purification of the Ps as shown in FIG. 11. As the ICGJ@PEI concentration in the inner water W₁ phase increased from 50 to 150 mg/mL, it increased monotonically up to 1.7 (FIG. 11). Note that I₈₉₅/I₇₈₀ ratio was only 0.4 for a low ICGJ@PEI level of 10 mg/mL (FIG. 12). Alternatively, simply by adding 5 mg/mL ICGJ to the outer W2 phase, I₈₉₅/I₇₈₀ ratio increased to 1.6 with only 50 mg/mL ICGJ@PEI in W₁ (FIG. 3C and Table 1). This condition was chosen as the base case for all other experiments in this study with all of the properties summarized in Table 1.

Further, the mechanism for the prevention of loss of J aggregates during encapsulation was described. Branched PEI in the non-protonated state is chloroform soluble. ICGJ@PEI may be suspended in chloroform for only seconds followed by settling. Upon redispersion of these ICGJ aggregates in water at pH 4, the ζ is ˜10 mV confirming that part of the PEI was removed by chloroform. During Ps formation, as ICGJ@PEI collides with the interface, it slowly loses the protective PEI layer to chloroform. The large charge of PEI at pH 4 gives stronger binding of the PEI to anionic ICGJ and decelerate the PEI loss relative to higher pH values, consistent with the results in FIG. 10A. Relative to ICGJ@PEI, unprotected ICGJ aggregates release surface active ICG molecules to saturate the water-chloroform interface more rapidly as discussed above. From the above analysis, all the ICG in ICGJ@PEI at 50 mg/mL in the W₁ phase would only cover about 84% of the W₁/O interface, Therefore, the significant dissociation of ICGJ aggregates observed at this concentration may be attributed to the loss of ICG to the water-oil interface (FIG. 11).

As the ICGJ@PEI concentration is raised to 150 mg/mL, three times as much ‘sacrificial’ ICG would be available to cover the W/O interfaces. Thus, a higher I₈₉₅/I₇₈₀ ratio during Ps formation was achieved as shown in FIG. 11. Similarly, the ICG may be provided by introducing 5 mg/mL ICGJ (without PEI) in the outer W₂ phase to serve as a concentrated source of ICG “sacrificial” monomer (again, the standard condition in this study). As this sacrificial ICGJ rapidly dissociates to ICG monomer at the W-O interfaces (as demonstrated in FIG. 4C), it saturates about 67% of the W₁/O interface in our standard condition for Ps formation (FIG. 13). The ICG monomer may be transferred from the W₂/O interface to the W₁/O interface by collisions of the W₁ droplets with the W₂/O interface and with each other and by diffusion through the oil phase. Other attempts that provided too little sacrificial ICG monomer were unsuccessful including the use of either ICG monomer or ICG@PEI in the W₂ phase as shown in FIG. 13. Thus, the need to supply sufficient ICG at the chloroform-water interface was a key requirement for preventing dissociation of J aggregates in the formation of Ps.

Next, although not bound by any particular theory, it was proposed that repulsive colloidal interactions of the ICGJ@PEI NPs may also contribute to inhibition of J aggregate dissociation at the interface. After enough of the cationic ICGJ@PEI NPs are attracted to the strongly negatively charged chloroform-water interface containing adsorbed anionic ICG, a local positive electric field repels oncoming cationic NPs, shielding them from the destabilizing interface. As the initial ICGJ@PEI concentration increases, a smaller fraction of these aggregates is needed to provide this type of electrostatic shielding, consistent with the highest I₈₉₅/I₇₈₀ ratio at 150 mg/mL (FIG. 11).

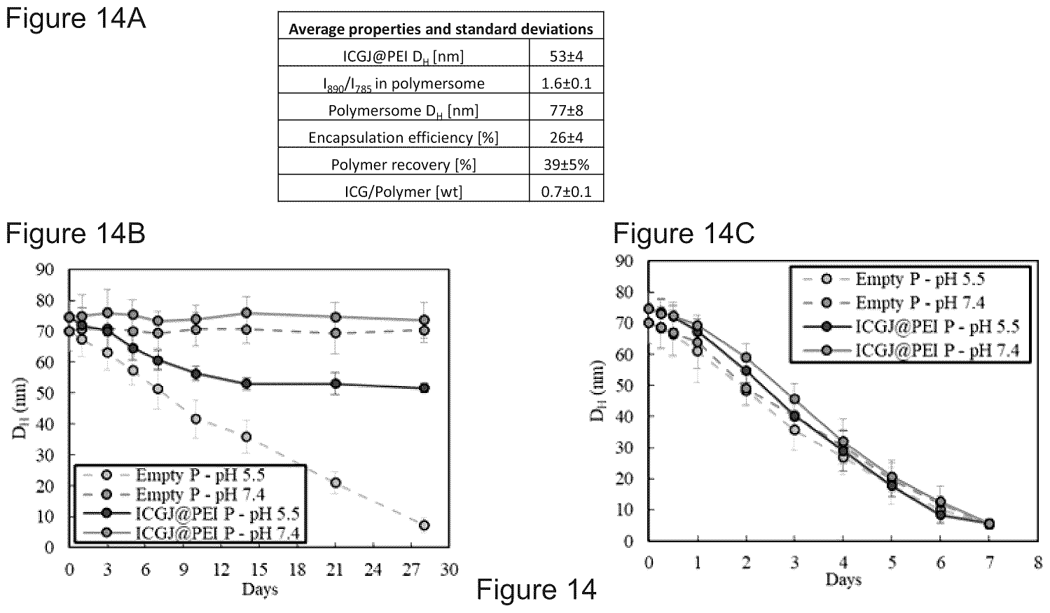

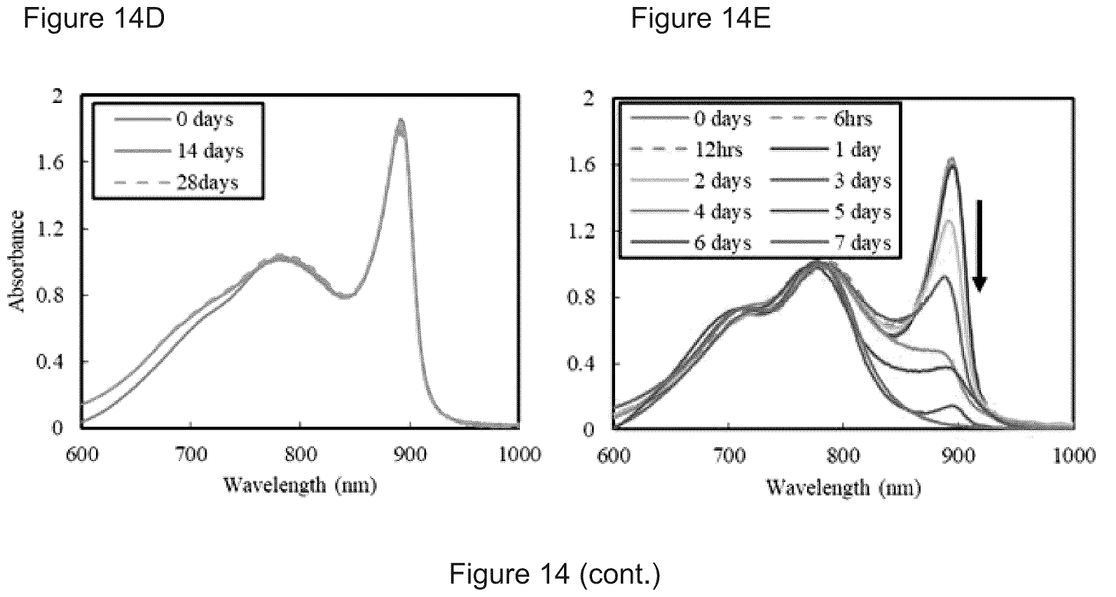

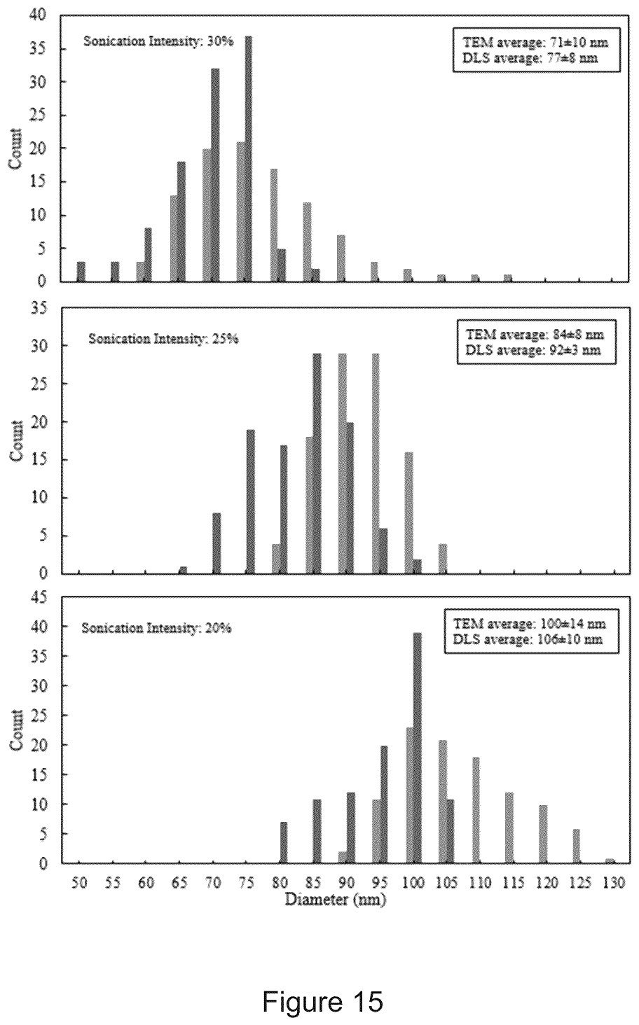

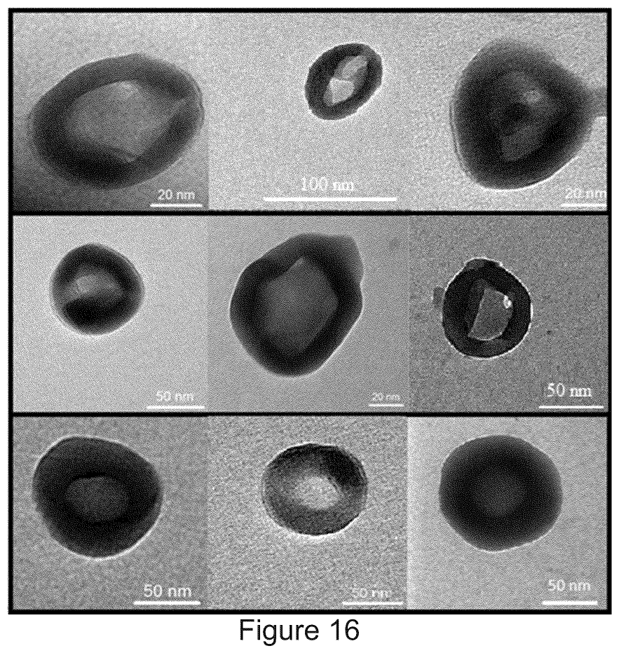

As shown by TEM, the Ps volume average diameter was 71 nm consistent with a volume average hydrodynamic diameter (DO of 77 nm by DLS (FIG. 10C, FIG. 14A, and FIG. 15). From TEM, a Ps shell (darker region) with a thickness of ˜15 nm surrounds the lighter ICGJ@PEI domain, which has a lower electron density (FIG. 10D and FIG. 16). The deviation from a spherical shape is likely an artifact of the drying process for TEM. This shell thickness is consistent with a D_{H} by DLS for 90 K PLGA block length of 16 nm in a good solvent acetone (https://akinainc.com/polyscitech/COA/PLGA-75L-H-180313RAI-B.pdf) and the D_{H} of free PEG-PLGA chains dispersed in chloroform, which ranged from 13 to 18 nm for a concentration of 25 mg/mL (Discher B M et al., 1999, Science, 284:1143-1146; Jang W S et al., 2015, Macromol. Rapid Commun., 36:378-384; Bermudez H et al., 2004, Langmuir, 20:540-543). An overlapping bilayer, with an interior layer of 90K PLGA chains with entanglement in the perpendicular and parallel directions relative to the interface, is likely to result in a tougher membrane in Ps than for conventional phospholipid vesicles based on previous studies of mechanical properties (Bermudez H et al., 2004, Langmuir, 20:540-543). For previously reported Ps composed of block copolymers of PEG-b-PLGA (3 k:9 k by mass), the shell thickness was nm (Yu Y et al., 2012, Pharmaceutical Research, 29:83-96). In our case with PEG-b-PLGA (10 k:90 k), the oxygen bearing carboxyl groups are weakly hydrated. Consequently, it behaves as if the PEG to PLGA mass ratio were higher, on the order of 0.2-0.3 in the range commonly studied for Ps composed of block copolymers with purely hydrocarbon highly hydrophobic blocks (Jain S et al., 2003, Science, 300:460-464; Blanazs A et al., 2009, Macromol. Rapid Commun., 30:267-277).

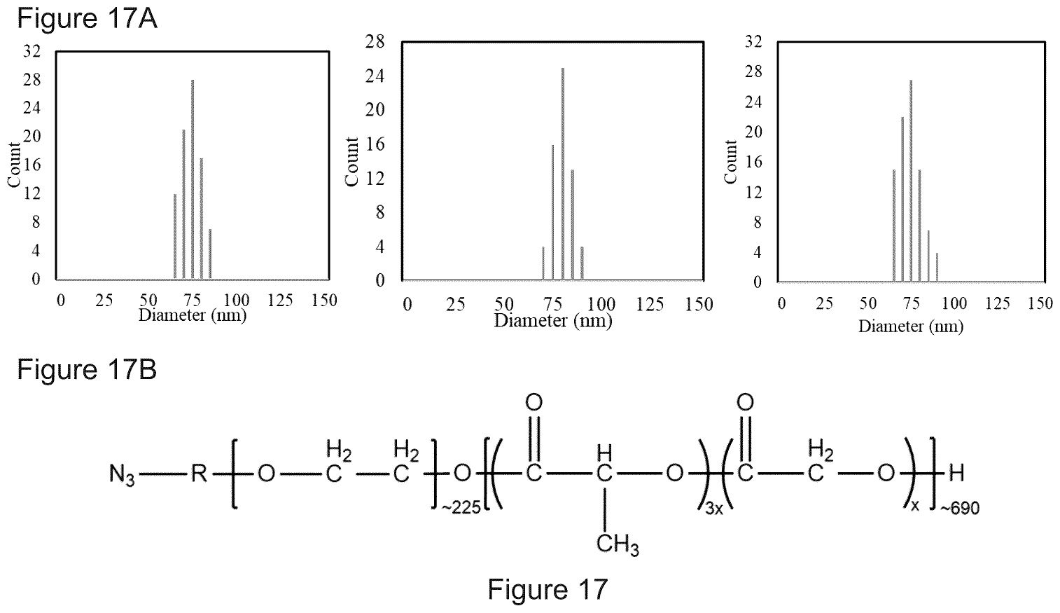

The amount of water between the ICGJ@PEI and inner Ps surface appeared to be small, but was not very distinguishable in TEM images due to a poor contrast associated with ICGJ aggregates. Note that the D_{H} of ICGJ@PEI was 53 nm (FIG. 10D and FIG. 7), not far below the inner diameter of empty Ps which have an effective outside diameter of 76 nm (FIG. 17A). Thus, ICGJ@PEI filled a very large fraction of the space in the Ps. No signs of unencapsulated ICGJ@PEI aggregates were detected in TEM images shown in FIG. 10D and FIG. 16 indicating that the purification with 10% ethanol was successful in dissociating the unencapsulated ICGJ@PEI aggregates, as expected from the control experiments in FIG. 2. Therefore, the much slower dissociation of the encapsulated versus unencapsulated J aggregates was sufficient to enable purification.

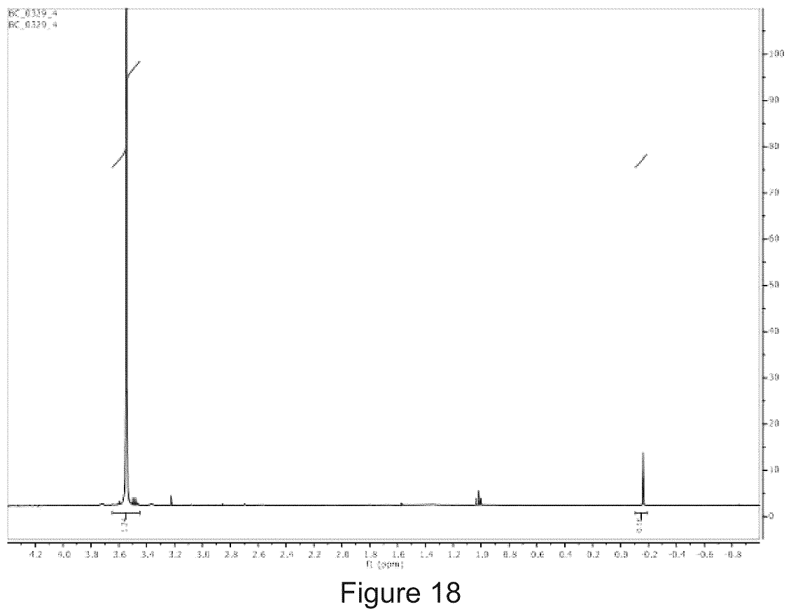

An ¹H NMR method was utilized for determining both the total and surface PEG content quantitatively (Xu Q et al., 2015, ACS Nano, 9:9217-9227), on the basis of peak broadening for the surface PEG at ˜3.65 ppm (FIG. 18) (Xu Q et al., 2013, Journal of Controlled Release, 170:279-286; Vila A et al., 2004, Journal of Controlled Release, 98:231-244). As shown in Table 2 ˜90% of the PEG chains were found to be on the inner and outer surfaces of the Ps. Assuming perfect spheres with smooth surfaces, the calculated surface PEG density was 2.4 PEG₁₀ₖ/100 nm² ([Γ][Γ*]=1.1), which is >1 indicating a brush-like configuration of PEG on the surface. The brush density is higher than typically achieved with surfactant-based liposomes given one PEG unit on each chain in the Ps bilayer (Bermudez H et al., 2004, Langmuir, 20:540-543).

The formation of a curved bilayer Ps morphology is due not only to the polymer block lengths and types, as has been seen in the case of self-assembly in an aqueous phase, but also to the formation process. Here, the process involves dewetting of the Ps by oil (FIG. 8) during evaporation of the oil in the W/O/W emulsion (Blanazs A et al., 2009, Macromol. Rapid Commun., 30:267-277; Hayward R C et al., 2006, Langmuir, 22:4457-4461; Shum H C et al., 2011, Journal of the American Chemical Society, 133:4420-4426). From DLS, during solvent evaporation, two peaks were observed in the volume distribution (FIG. 3D and FIG. 19). The D_{H} of the larger red peak representing the oil droplets started at 460 nm and shrunk to 270 nm in six hours. The volume fraction of this smaller particle blue peak is shown by the black line that is labelled on the right side y axis in FIG. 3D. After an hour, the smaller peak was below 150 nm, but reached a steady state value of ˜70 nm after three hours. Given that this is the size of the final Ps, the smaller peak may be assigned to be primarily Ps. (It is unlikely that the red peak is aggregated Ps given the large early volume fractions and that is not expected that aggregated Ps would become deaggregated without high shear forces.) Although not bound by any particular theory, it was proposed that a likely mechanism consistent with these results that is based on a previous Ps study where only a single water droplet was present in each oil droplet given the use of microfluidics (Hayward R C et al., 2006, Langmuir, 22:4457-4461). During dewetting, the inner (W₁/O) and outer (O/W₂) water-oil interfaces, with the associated polymer layers, merge and are “zipped” together resulting in a bilayer film driven by the work of adhesion Wadh between the interfaces (FIG. 8C). The Wadh depends upon the contact angles for the inner and outer films and the interfacial tensions, which are influenced by the interfacial compositions of the surfactants including PEG-PLGA and IGC, as well as the rate of solvent evaporation. This mechanism is further supported by studies of the effect of sonication intensity. As shown in FIG. 15, the average D_{H} of the Ps decreased from 106 to 77 nm as the sonication intensity was raised from 20% to 30% to increase the shear forces. Ultrasonic energy produces finely dispersed W₁/O emulsions by creating turbulent flow conditions on a macroscopic and a microscopic scale (Tal-Figiel B et al., 2007, Chemical Engineering Research and Design, 85:730-734). As may be expected, smaller Ps are formed when smaller water droplets are dewetted by oil as they leave the shrinking oil droplets (FIG. 8).

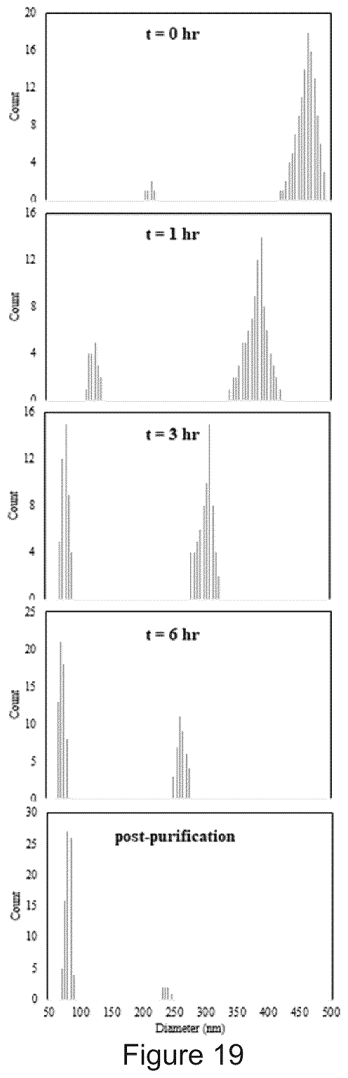

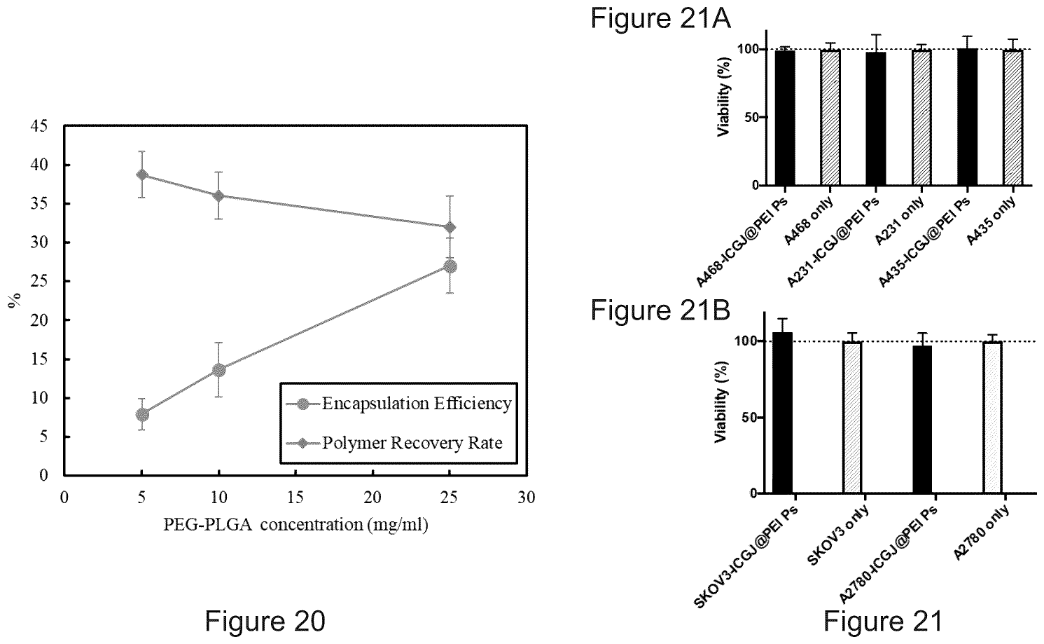

The encapsulation efficiency (EE) of ICG=(encapsulated ICG (mg)÷input ICG (mg)) 100, was calculated by determining the ICG concentration from the spectral calibration curve, as explained in the experimental section. The EE was 26±4%, which is a reasonable value for the W/O/W process (Saxena V et al., 2004, Journal of Photochemistry and Photobiology B: Biology, 74:29-38; Patel R H et al., 2012, Journal of Biomedical Optics, 17: 046003). As shown in FIG. 20, the EE of ICG increases with a higher PEG-PLGA concentration in the oil phase during encapsulation, which increases the number of Ps for trapping the ICGJ@PEI NPs. The average recovery rate of PEG-PLGA polymer in the Ps was 39±5% (FIG. 14A), and the average ratio of ICG to polymer was 0.7±0.1 (w/w). For a known Ps diameter of 80 nm, and an assumed shell thickness of 10 nm and the polymer density, the fraction of Ps occupied by one ICGJ@PEI would be 0.7. Since PLGA biodegrades, the presence of empty Ps should not have any adverse biological effects in vivo. In summary, Ps were formed with a D_{H} of 77 nm and an interior nearly fully occupied with ICGJ@PEI as summarized in FIG. 14A. With a large fraction of the ICG in the J aggregate state the I₈₉₅/I₇₈₀ ratio of 1.6 was relatively high, given the use of sufficient “sacrificial ICG” and further protection by electrostatically attached PEI.

### Stability and Dissociation of the Polymersomes

Ideally, the Ps must preserve the spectral properties for ˜24 hours for PAI and then biodegrade for clearance from the body. The stability of ICGJ NPs in DI water and 100% FBS at pH 5.5 (simulating the pH of intracellular endosomal compartments) and 7.4 (physiological pH) was first determined. ICGJ NPs as well as ICGJ@PEI NPs are completely stable in DI water via strong electrostatic repulsion against aggregation and dissociation with no discernable change in D_{H} at both pH values after two weeks (FIG. 7). However, ICGJ NPs completely dissociated in 100% FBS in less than 10 minutes at both pHs as did ICGJ@PEI NPs in about 30 minutes. The slight protection by PEI may be attributed to electrostatic attraction of the anionic ICG molecules with the cationic PEI sites in the shell. However, the ICG still escaped the J aggregates readily as PEI cannot bind to all of the ICG molecules on the ICG J aggregate surface.

The J aggregates and Ps remained stable in water at pH 7.4 and at 4° C. for 28 days with a change in D_{H} and I₈₉₅/I₇₈₀ absorbance ratio of less than 2% (FIG. 14B and FIG. 14D) that continued for 90 days. Thus, the dense PEG inside layer in the Ps bilayer, which interacted weakly with the internal space filling ICGJ@PEI aggregates, provided protection against the PLGA part of the bilayer. This observation is further supported by the fact that aqueous solutions of PEG do not destabilize ICGJ, whereby the spectra remained constant for at least 1 day (FIG. 4A). However, at pH 5.5, the D_{H} of the ICGJ@PEI Ps solution decreased to −50 nm after two weeks, and then stayed constant for up to 4 weeks, with little change in the spectra (FIG. 14D) (Holy C E et al., 1999, Biomaterials, 20:1177-1185). In contrast, D_{H} of empty Ps without any J aggregates continued to decrease in size down to 10 nm as the PLGA in the Ps walls hydrolyzed. Thus, these steady state 50 nm particles in FIG. 14B may be identified as released ICGJ@PEI NPs from the degraded Ps, which were shown above to be stable at pH (FIG. 7).

The dissociation behavior of the Ps in 100% FBS was quite different compared to DI water. The D_{H} of ICGJ@PEI Ps decreased from 78 to 65 nm, at pH 5.5 and 7.4, in the first two days and then decreased more rapidly to below 10 nm after 7 days (FIG. 14C). Any ICGJ@PEI released from the Ps in 100% FBS would dissociate within minutes as shown in FIG. 7; therefore, the D_{H} represented the size of the Ps and not that of ICGJ@PEI. To further support this concept, the D_{H} kinetics were similar to those for the empty Ps (FIG. 14C). The faster dissociation rate in FBS compared to DI water at pH 5.5 may be attributed to enzyme-catalyzed hydrolysis of PLGA (Cai Q et al., 2003, Biomaterials, 24:629-638). The long 10K PEG chains can facilitate the enzymatic attack significantly by increasing wetting of the PLGA shell (Li S et al., 2000, Polymer Degradation and Stability, 71:61-67). Also, the increase in the degradation rate after the first two days most likely reflects autocatalysis caused by acidification in the shell from released lactic acid monomer (Park, T G et al., 1995, Biomaterials, 16:1123-1130). In 100% FBS, the I₈₉₅/I₇₈₀ changed relatively little over the first day (FIG. 14E), and then decreased over 7 days as the D_{H} of the Ps became very small (FIG. 14C). This behavior is desirable for performing photoacoustic imaging in vivo to ensure stability during intravenous administration. After determining that ICGJ@PEI Ps had a long shelf life in DI water and 24 hour stability in 100% serum, we evaluated their potential cytotoxicity in live cells and validated molecular specific PAI in breast and ovarian cancer cells.

### Cellular Response to ICGJ@PEI Ps and Molecular Specific Photoacoustic Imaging

To determine if ICGJ@PEI Ps have cytotoxicity due to the surface coating such as PEI or the nature of dissociation of the Ps, quantitative cell viability assay (MTS) was performed with five different breast and ovarian cell lines (FIG. 21). There were no statistically significant differences in viability between cells incubated with Ps and untreated controls, indicating biocompatibility of Ps.

Ps were conjugated with monoclonal antibodies specific to epidermal growth factor receptor (EGFR) (Barnes C J et al., 2004, Cancer Treat. Res., 119:1-13) for molecular targeting of cancer cells. EGFR is a biomarker of one of the major cancer hallmarks—“sustaining proliferation signaling” (Hanahan D et al., 2011, Cell, 144:646-674). This receptor tyrosine kinase is overexpressed in many malignancies including ovarian (Barnes C J et al., 2004, Cancer Treat. Res., 119:1-13) and triple negative breast cancers (Foulkes W D et al., 2010, N. Engl. J Med., 363:1938-1948). Anti-EGFR antibodies were attached to azide groups on the surface of ICGJ@PEI Ps using a directional copper-free click chemistry. This conjugation was based on a modification of an approach developed by us where antibodies are bound through carbohydrate moieties on their Fc portion leaving the Fab fragment available for efficient binding of an antigen (Kumar S et al., 2008, Nat. Protoc., 3:314-320). The D_{H} of ICGJ@PEI Ps-antibody conjugates (ICGJ@PEI Ps-Ab) was ˜89±8 nm with ζs −1.7±1.6, −4.2±3.9, and −10.6±3.6 pH 4 and 7 and 10, respectively (FIG. 3A). It was determined that ˜11 antibodies were attached per ICGJ@PEI Ps (see details in experimental section below).

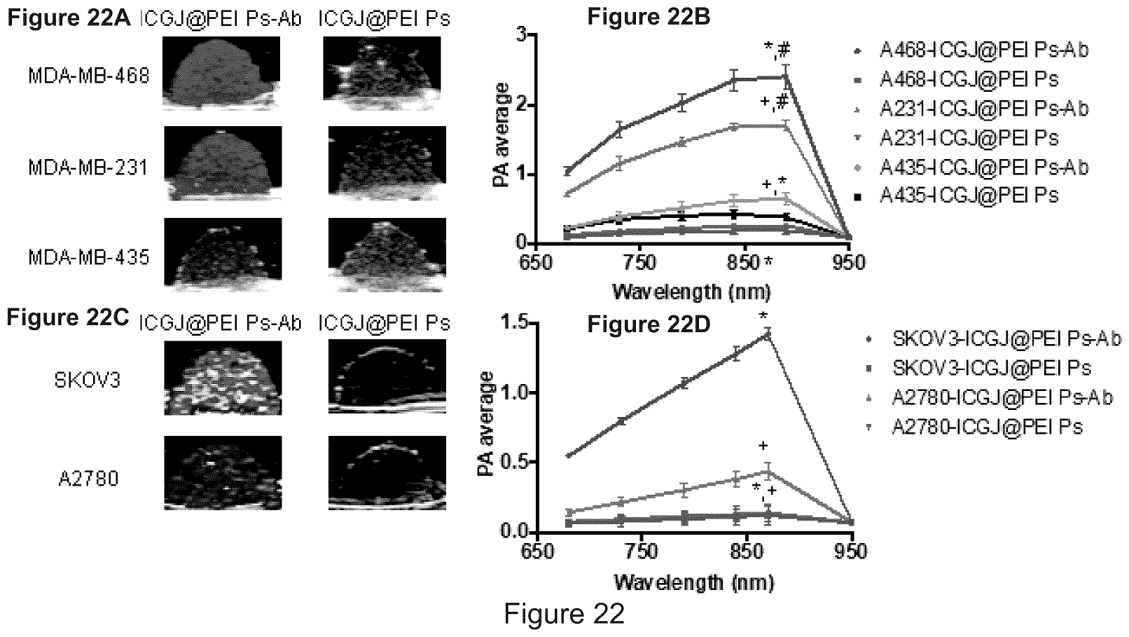

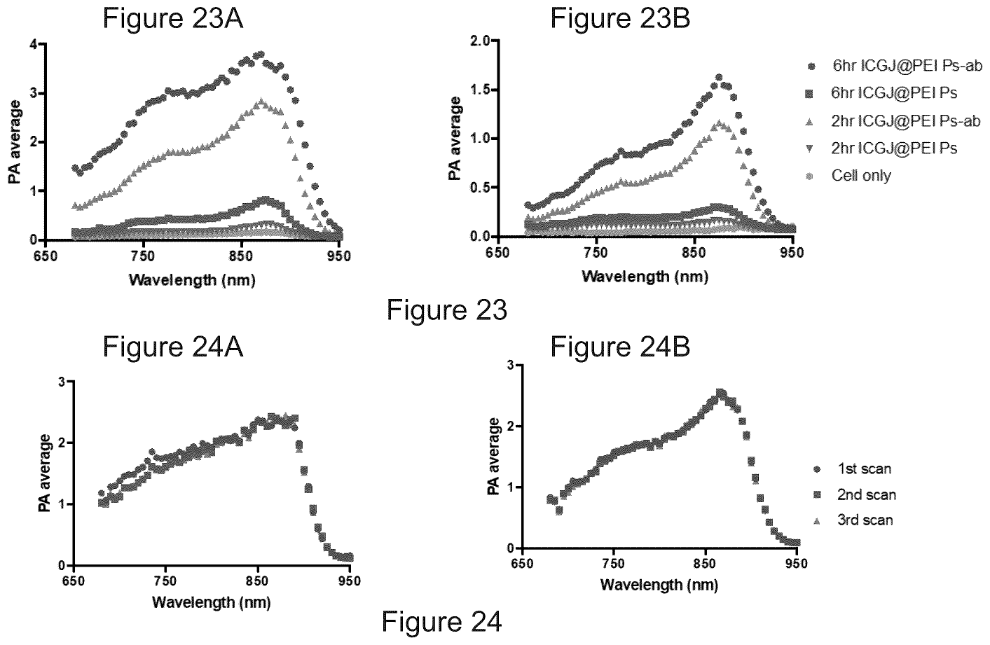

Molecular PAI was evaluated in three breast and two ovarian cancer cells. Triple-negative breast cancer cell lines MDA-MB-468, MDA-MB-23 land MDA-MB-435 with high, intermediate and no EGFR expression, respectively, were used (Chavez K J et al., 2010, Breast Dis., 32:35-48; Subik K et al., 2010, Breast Cancer (Auckl), 4:35-41). In addition, SKOV3 and A2780 ovarian cancer cells with high and low EGFR expression levels, respectively, were evaluated (Gottschalk N et al., 2012, Int. J. Mol. Sci., 13:12000-12016). All cells labeled with EGFR-targeted ICGJ@PEI Ps exhibited photoacoustic (PA) signals that correlated with their inherent EGFR expression levels (FIG. 22) (Chavez K J et al., 2010, Breast Dis., 32:35-48; Subik K et al., 2010, Breast Cancer (Auckl), 4:35-41; Gottschalk N et al., 2012, Int. J. Mol. Sci., 13:12000-12016). The highest PA signal appeared around 873 nm near the region of the ICGJ aggregate peak. For breast cancer, labeled MDA-MB-468 cells had ˜1.5-fold and ˜2.2-fold stronger PA signal than MDA-MB-231 and MDA-MB-435, respectively, at the 870 nm excitation (FIG. 22A and FIG. 22B). SKOV3 labeled cells exhibited ˜3.3-fold higher signal as compared to A2780 cells (FIG. 22C and FIG. 22D). Cell labeling over two time periods of 2 and 6 hours resulted in nearly the same PA signal ratio for SKOV3 and A2780 cells of 2.44 and 2.33, respectively, indicating that incubation time did not have a strong effect on the relative PA signal (FIG. 23). There was a minimum background signal after incubation with non-targeted ICGJ@PEI Ps (FIG. 22). Further, PA signal of labeled cells was stable over time with no changes observed after continuous acquisition of three consequent spectroscopic PA measurements with 5 nm resolution in the 680-950 nm range (FIG. 24).

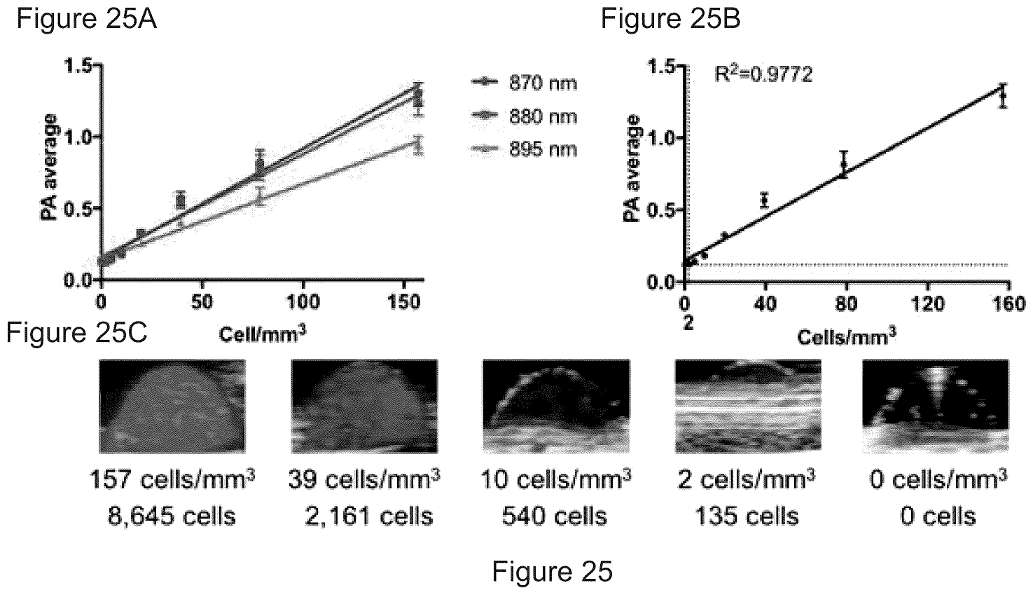

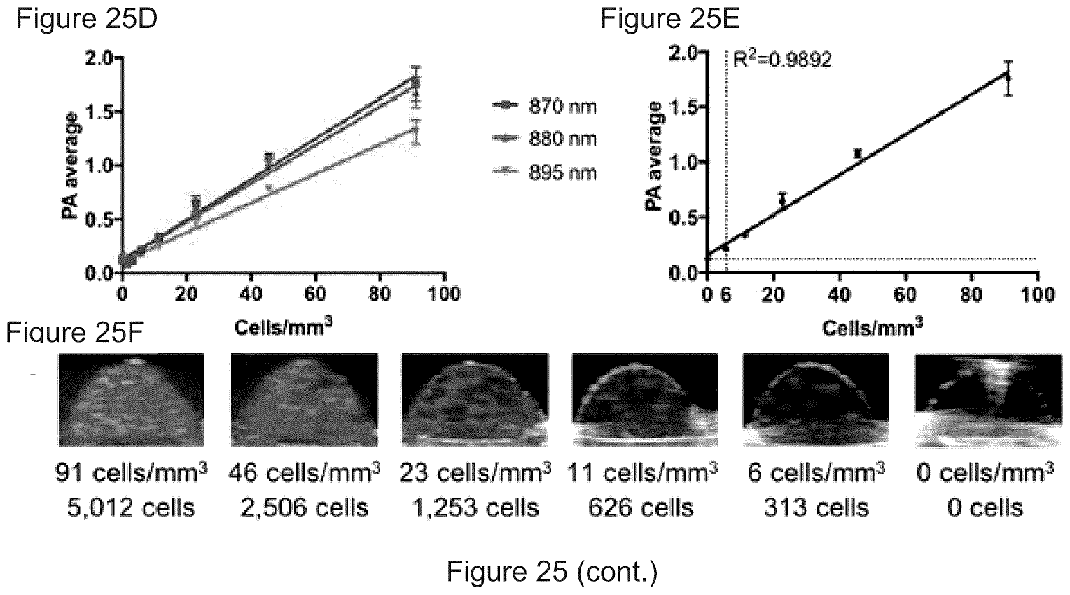

Sensitivity of PAI with ICGJ@PEI Ps-Ab in detection of MDA-MB-468 and SCOV3 cancer cells was determined in tissue mimicking phantoms using serial dilutions of pre-labeled cells (FIG. 25). Linear regression fits at 870 nm showed detection limits of labeled MDA-MB-468 and SKOV3 cells as low as 2 and 6 cells/mm 3 that corresponds to the total amount of 135 and 313 cells/well, respectively. This cellular PA detection limit is at least ˜10 fold better than previously reported in the literature (Qin X et al., 2018, Adv. Funct. Mater., 28:1-23; Mallidi S et al., 2009, Nano Lett., 9:2825-2831; Lavaud J et al., 2017, Int. J. Pharm., 532:704-709). For example, human embryonic stem cell-derived cardiomyocytes prelabeled with a NIR-absorbing 50 nm NPs composed of semiconducting polymers were detected with sensitivity of ˜2000 cells following subcutaneous injection under skin of a mouse (Qin X et al., 2018, Adv. Funct. Mater., 28:1-23). In another study, PAI of A431 keratinocytes labeled with 50 nm spherical AuNP targeted to EGFR showed a detection limit of ˜310 cells/mm 3 (Mallidi S et al., 2009, Nano Lett., 9:2825-2831; Lavaud J et al., 2017, Int. J. Pharm., 532:704-709). Further, B16F10 melanoma cells were detected using their endogenous PA signal from melanin at a concentration of ˜6,250 cells/mm 3 (Lavaud J et al., 2017, Int. J. Pharm., 532:704-709). Thus, these results indicate that ICGJ@PEI PS-Ab show a substantially superior performance in generating a strong PA signal in labeled cells as compared to previous cellular PAI studies.

The strong NIR absorbance of ICGJ encapsulated within PEG-PLGA Ps and their stability in various media including 100% FBS enabled PAI of breast and ovarian cancer cells with high specificity and sensitivity. The successful encapsulation of stable ICGJ into polymersomes was achieved by mitigating shedding of interfacially active ICG molecules from the J aggregates to the oil-water interfaces in the W/O/W double emulsion, upon supplying a sufficient level of “sacrificial” ICG monomer. Furthermore, the inner PEG layer of the Ps shell and the PEI coating protected the ICGJ@PEI against dissociation during Ps purification and long-term storage. During shrinkage of oil droplets in the process of evaporation, the concentrations of the two amphiphilic molecules ICG and PEG-PLGA, as well as the interfacial and colloidal properties, apparently were at appropriate conditions for release of 77 nm relatively monodisperse Ps, via dewetting from the oil phase. The thick Ps bilayer stabilized the J aggregates and the dense PEG brush layer provided steric stabilization and colloidal stability even in 100% FBS for ˜24 hours, a sufficient time for in vivo applications with systemic delivery. All of the components in the system are fully biodegradable, and therefore expected to have favorable body clearance characteristics. PAI of breast and ovarian cancer cells labeled with EGFR-targeted ICGJ Ps showed a high specificity and an impressive sensitivity at the level of ˜100 total cells at a few millimeters depth. This high sensitivity can be associated with a number of factors including (i) fluorescence quenching in J aggregates that increases absorbance, (ii) a very high loading efficiency of ICG per Ps with an ICG/polymer weight ratio of ˜0.7 (FIG. 14A), and, potentially, (iii) a nonlinear PA signal enhancement that can result from densely packed photoabsorbers (Nam S Y et al., 2012, Opt. Lett., 37:4708-4710). Furthermore, all major components of the Ps are FDA approved and are used in the clinic including (i) ICG dye in surgical visualization, (ii) PLGA polymers in multiple drug formulations and (iii) humanized anti-EGFR cetuximab antibodies in cancer treatment; this can greatly facilitate clinical translation of the Ps described herein. In addition, the Ps formation process is quite general and may be utilized to encapsulate a wide variety of agents including imaging agents and pharmaceuticals in theranostics.

The materials and methods employed in these experiments are now described.

Materials

Indocyanine green (ICG) was purchased from Tokyo Chemical Industry (TCI America) Co. Ltd. Polyethylenimines (PEI) (MW: 1800-branched, 2000-linear, 10000-branched) were purchased from Alfa Aesar, chitosan (MW: 100000-300000) from Arcos Organics, and low molecular weight chitosan (Mw: 1000) from Carbosynth. Poly(ethylene glycol) (PEG)=500-10000) was purchased from Creative PEGWorks along with custom synthesized Azide-poly(ethylene glycol)-b-poly(lactide-co-glycolide) (A-PEG-PLGA) (M_{w}: 100000, PEG 10k and PLGA 90k, L:G 75:25). Poly(vinyl alcohol) (PVA) (M_{w}: 13000-23000, 88% hydrolyzed), stearic acid, arachidic acid, and poly(allylamine hydrochloride) (PAH) (M_{w}: 17500) were purchased from Sigma Aldrich. Poly(acrylic acid) (PAA) (M_{w}: 25000, 25% wt solution in water) was purchased from Polysciences Inc. Acetone, Ethanol, diethyl ether, dichloromethane (DCM), dimethylformamide (DMF), fetal bovine serum (FBS), calcium chloride dihydrate (CaCl₂·2H₂O), and sodium citrate (NaCit) were bought from Fisher Scientific Co. and D₂O from Cambridge Isotope Laboratories Inc.

Preparation of ICG J Aggregates (ICGJ) and Coating with a PEI Shell to Form ICGJ@PE 1.5 mM (1.15 mg/mL) ICG aqueous solution was heated in an oil bath at 65° C. while stirred at 750 rpm using a rod-shaped magnetic stir bar for 24 hours and the formation of ICGJ was monitored with DLS and UV-vis-NIR-NIR spectroscopy. After 24 hours, 6 mL ICGJ solution was sonicated with ultra-sonication probe for 1 min/mL at 15% output power (model: Branson™ S-450 Digital Sonifier, probe length: 16 cm, tip diameter: 0.3 cm) and purification was performed. To coat the anionic ICGJ NPs covered with a cationic PEI shell, (100 μL of 10 wt %) PEI solution was added to (˜0.5 mL) solution of (˜50 mg/mL) ICGJ and the pH was set to 4 with dropwise addition of (1 N HCl) to nearly fully protonate the PEI. The mixture was sonicated with a bath-sonicator (model: Fisherbrand™ CPXH series model 2800) for 5 minutes. and further stirred at room temperature for 2 hours and purified.

Characterization of ICGJ and ICGJ@PEI NPs

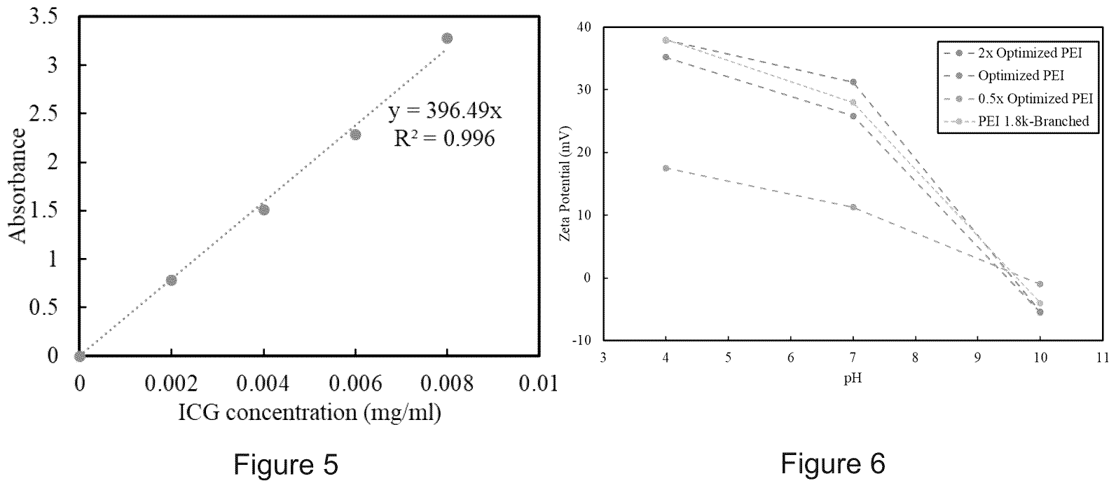

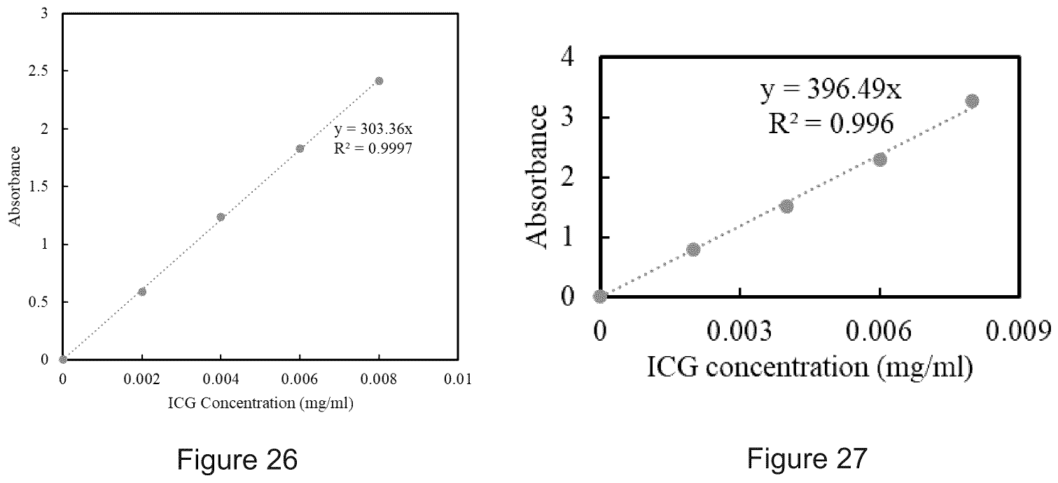

The ICGJ or ICGJ@PEI NP dispersions were diluted to (˜0.05 mg/mL) and filtered through a 200 nm poly(ether sulfone) (PES) syringe filter prior to performing dynamic light scattering DLS, zeta potential, and UV-vis-NIR measurements. Volume average diameters were measured to within 10% on a Brookhaven ZetaPALS instrument using a 90° detection angle. The zeta potentials were measured in the ZetaPALS mode assuming a Huckel model. A minimum of 6 runs were performed for each sample with 10 cycles with an average applied electric field of ˜9 V/cm. For UV-vis-NIR spectroscopy the solution was mixed with an equal volume of ethanol and the ICG concentration was determined with a Varian Cary 60 spectrophotometer with a path length of 1 cm on the basis of a standard concentration-OD curve (FIG. 26 and FIG. 5). This calculation was further confirmed by total organic carbon (TOC, Shimadzu TOC-LCPH/CPN) measurements. The PEI content in the purified ICGJ@PEI composite was determined by TOC.

Encapsulation of ICGJ@PEI in PEG-PLGA Ps

Azide-PEG-PLGA Pss were prepared using a double emulsion (W/O/W emulsion) process as shown in FIG. 8 (Gryparis E C et al., 2007, European Journal of Pharmaceutics and Biopharmaceutics, 67:1-8). The oil phase was prepared by dissolving A-PEG-PLGA (25 mg, 100 k 75:25 L/G) in chloroform (1 mL). The inner water phase was (500 μL) of (˜50 mg/mL) ICGJ@PEI solution. The first emulsion (W/O) was formed under sonication using a Branson™ S-450 Digital Sonifier probe sonicator for 6 times (10 seconds each, amplitude 30%). To generate a double emulsion (W/O/W), the W/O emulsion was added within 2-3 seconds to (4 mL) aqueous solution of (5 mg/mL) ICGJ (to reduce the dissociation of ICGJ@PEI in the inner water phase) and PVA (5 wt %, average MW 18000), followed by sonication for 6 times (10 seconds each, amplitude 30% gaps of 5-10 seconds for cooling) (Ashjari M et al., 2012, Colloids and Surfaces A: Physicochemical and Engineering Aspects, 408:87-96). The rapid mixing was needed since the water droplets in the W/O emulsion settled in a period of about 1 minute. Afterwards, the double emulsion was diluted in (40 mL) DI water and stirred magnetically with a rod-shaped stir bar at room temperature overnight to evaporate the chloroform and form the capsules.

For purification, (4 mL) of polymersomes were diluted with (9.5 mL DI) water in a 15 mL plastic tube and centrifuged 3 minutes at 5000 rpm to remove large aggregates of insoluble PEG-PLGA as a pellet. The supernatant was decanted and incubated for 10 minutes with (10% v/v) ethanol/water solution and then centrifuged three times using Amicon Ultra-15 kDa MWCO centrifugal filters with 15 mL capacity (regenerated cellulose membrane, Millipore Sigma) at 5000 rpm for 15 minutes each cycle ((5 mL) aliquots of the Ps sample were mixed with (8.5 mL) of DI water and (1.5 mL) of ethanol, making a 10% ethanol solution). The purpose of these three cycles was to dissociate ICGJ@PEIs to very small ICG micelles that are able to permeate the filter. Control experiments showed that purified ICGJ@PEI aggregates within PLGA-PEG capsules undergo only modest dissociation in 15 minutes in 10% v/v ethanol/water solution based on the UV-vis-NIR-NIR spectra. In contrast, the unencapsulated ICGJ@PEI dissociates completely under these conditions (FIG. 2). During completion of the third round of diafiltration with the centrifugal filter, the 5 mL Ps aliquot was concentrated to a volume of (1.5 mL). This dispersion was purified by centrifugation at 30000 rpm for 30 minutes and rinsing with DI water for 3 times. After each round of ultracentrifugation, the formed pellet containing Ps was re-dispersed in DI water to its original volume (1.5 mL) and then probe sonicated (with an intensity of 15%) for 10 seconds. The encapsulation efficiency and polymer recovery were determined as shown belowa. To characterize the degradation of the capsules, (1 mL) of a (0.05 mg/mL) Ps solution was mixed with (1 mL) of DI water and the pH was adjusted daily to 5.5 with 0.1 N HCl or was maintained at pH 7.4.

Synthesis of ICGJ@PEI Ps Antibody Conjugates

ICGJ@PEI Ps were conjugated with monoclonal anti-EGFR antibodies (clone 225, Sigma, E2156) to confer EGFR-targeting specificity. Antibodies were first purified using a 100 kDa MWCO centrifuge filter (EMD Millipore) and reconstituted in (100 mM) sodium phosphate buffer, pH 7.5. Then, one hundred μL of (100 μg/mL) antibody solution was mixed with (10 μL) of (100 mM) sodium periodate (Sigma-Aldrich) in the dark for 30 minutes at 350 rpm at room temperature (RT). During this step, the adjacent hydroxyl groups on antibody's carbohydrate moiety are converted into aldehydes through a mild oxidation process (Kumar S et al., 2008, Nat. Protoc., 3:314-320). The reaction was quenched by (500 μL) PBS. Then, (2 μL) of bifunctional DBCO-PEG-aminooxy linker (M.W. 3.4 kDa, PG2-AODB-600, NanoCS) at 49 mM in DMSO (Thermoscientific) was added to the oxidized antibodies, and the solution was kept on a shaker at 350 rpm for 1 hour at RT. Unreacted linkers were removed by three washing steps using a 30 kDa MWCO centrifuge filter (EMD Millipore) at 3100 g for 15 minutes at 4° C. The washed linker-antibody conjugates were reconstituted in PBS. Subsequently, (242 μL) of antibody-linker molecules at (100 μg/mL) in PBS was added at a 34-fold molar excess to (618 μL) of azide-functionalized ICGJ@PEI Ps (1.23 mg/mL of PEG-PLGA concentration in the polymersomes) in DI water. The mixture was incubated for 6 hours at 37° C. Excess antibodies were removed by two centrifugations of the ICGJ@PEI Ps antibody conjugates (ICGJ@PEI Ps-Ab) at 12000 g for 20 min. The final antibody-conjugated polymersomes were reconstituted in PBS.

Cell Culture

All cells were cultured in a humidified atmosphere and 5% CO2 at 37° C. Human ovarian carcinoma SKOV3 and A2780 cells were maintained in RPMI 1640 supplemented with 15% fetal bovine serum and 0.1% gentamicin sulfate (Gemini Bioproducts). Human breast carcinoma MDA-MB-468, MDA-MB-231, and MDA-MB-435 cells were grown in DMEM-F12 supplemented with 10% fetal bovine serum and 1% penicillin-streptomycin (Life Technologies). Cells were sub-cultured every 2 days (prior to reaching 80% confluence) using standard techniques.

Tissue-Mimicking Phantom Preparation and Cellular Photoacoustic (PA) Imaging

Cellular PA imaging was conducted in a tissue-mimicking gelatin phantom (Cook J R et al., 2011, Biomed. Opt. Express, 2:3193-3206) containing 36 inclusions with a semi-ellipsoidal shape and (˜57 μL) volume capacity. For phantom preparation, 8% w/v gelatin (Sigma, G9382) in water was mixed with 1% v/v propanol and 0.1% v/v glutaraldehyde (Sigma-Aldrich) at 45° C., and the mixture was poured into a polydimethylsiloxane (PDMS, Dow Corning) mold with the inclusions and cooled down to 4° C. The resulting cross-linked gelatin was then removed to serve as a tissue-phantom background. Cells were seeded at 500000 cells/well in a 6-well plate and incubated overnight. ICGJ@PEI Ps-Ab nanoparticles were then added at (˜21 μM) (i.e., ICG concentration in the polymersomes) per well and incubated for 6 hours at 37° C. After incubation, unbound particles were washed using cell culture media, and cells were collected by centrifugation at 150 g for 10 min. Cell pellets were reconstituted in (˜100 μL) of 4% v/v paraformaldehyde solution in PBS (Electron Microscopy Sciences) and were counted using a hemocytometer. Then, cell suspensions at a desired concentration were mixed with 16% w/v gelatin in a 1:1 v/v ratio. The gelatin/cell suspensions were decanted into inclusions in the background gelatin phantom in triplicate, and a gelatin layer of ˜3 mm thickness was deposited over top. A Vevo® LAZR high-frequency ultrasound and photoacoustic imaging system (VisualSonics) equipped with a liner array transducer (LZ-250, 21 MHZ center frequency) was used for PA imaging. An optical parametric oscillator (OPO) laser pumped with an Nd:YAG laser was used to generate 5 ns pulses at a 20 Hz repetition rate. For volumetric photoacoustic measurements, B-mode ultrasound was acquired in parallel with spectroscopic PA imaging at 680, 730, 790, 840, 890, and 950 nm at a 0.47 mm step size. Volumetric segmentation of B-mode ultrasound images was used to define regions of interest (ROI) in calculations of PA signal intensities. All reported PA intensities were based on three independent but matched samples at the same dynamic range.

### ICGJ

Purification of ICGJ

The ICGJ solution was filtered through a 0.22 μm syringe filter to remove any possible large aggregates and then dialyzed (cellulose membrane, MW cut-off 3 kDa, Sigma) against DI water for 18 hours, changing the DI water every 6 hours to remove any ICG monomer permeate. To achieve the desired concentration of ICGJ, the solution was centrifuged with 30 kDa MWCO centrifugal filters at 5000 rpm for ˜2 hours.

### ICGJ@PEI

Purification of ICGJ@PEI

After forming ICGJ@PEI, the solution was mixed with 0.1 mL ethanol for 2 minutes to fully dissociate any uncoated ICG J aggregates, based on data in FIG. 2. The dissociation on ICGJ@PEI was minimal for this exposure time given the electrostatic stabilization with the PEI shell. Finally, the solution was washed and centrifuged with 30 kDa MWCO centrifugal filters (regenerated cellulose membrane) with 15 mL capacity at 5000 rpm 3 times with 10 mL DI water to remove the free ICG and PEI chains in the solution.

Characterization of ICGJ and ICGJ@PEI NPs

For DLS, the ACFs were fit with a non-negatively constrained least-squares multiple pass (NNLS) method and an autofit slope analysis option. The autocorrelation functions had flat initial and final baselines and were fit automatically for all particles where the first peak was smaller than 200 nm.

Stripping of PEI from ICGJ@PEI NPs

0.5 mL of ICGJ@PEI 50 mg/mL were air dried and suspended in 1 mL chloroform. The suspension was sonicated 5 times at 30% intensity for 5 seconds each cycle. The suspension settled from the chloroform phase in a few minutes, as the PEI desorbed and the underlying ICGJ aggregates became negatively charged. The aggregates were dissolved in water at pH 4 and analyzed by UV-vis-NIR spectroscopy and the zeta potential.

Description of Shell Morphology

Calculation of PEI Shell Thickness per ICGJ NP after Purification of ICGJ@PEI NPs

The input mass of ICGJ NPs (M_{ICGJ, feed}=22 mg, obtained from UV-vis-NIR after 3× washing of ICGJ@PEI NPs) and the ICGJ average diameter (D_{ICGJ}=50 nm, from hi-res TEM) was used to calculate the volume of one ICGJ NP:

$${V}_{icgj} = \frac{4}{3} π {R}_{icgj}^{3} .$$

From the calculated volume of a single ICGJ, and the density of ICGJ, the mass of a single ICGJ NP can be calculated. The density of ICGJ (ρ_{ICGJ}) is 962 mg/cm³, which is the hexagonal close packed factor (0.74) multiplied by the bulk density of ICG. So, M_{icgj}=V_{icgj}×ρ_{icgi}. By dividing the input mass of ICGJ by the mass of a single ICGJ, the number of ICGJ NPs can be calculated. Here, it is assumed that all ICG is in aggregated form as evidenced by the spectra of ICGJ@PEI NPs shown in FIG. 3C:

$${\#}_{icgj} = \frac{{M}_{icgj , feed}}{{ρ}_{icgj} \times {V}_{icgj}} .$$

Based on the input values, the number of ICGJs is about 3.5×10¹⁴.

To calculate the thickness of the PEI shells, the amount of PEI remaining in the system (M_{pei,feed}=5.4 mg, measured from TOC) and the density of PEI (ρ_{PEI}=1.08 g/mL) is needed. By dividing the remaining PEI mass by density, the volume of PEI available to coat the ICGJ NPs was determined:

$${V}_{pei , available} = \frac{{M}_{pei , feed}}{{ρ}_{pei}} .$$

From the equation above, there is 5×10¹⁸ nm³ PEI available to coat ICGJ NPs. Assuming that PEI covers all ICGJ NPs uniformly, a shell thickness can be estimated for ICGJ@PEI NPs. As shown above, the number of ˜50 nm ICGJ NPs in 0.5 mL of 44 mg/mL solution is equal to 3.5×10¹⁴. Therefore, the volume of a PEI layer around a single ICGJ NP is:

$${V}_{pei} = \frac{5 \times {10}^{18} {nm}^{3}}{3.5 \times {10}^{14}} = 1.43 \times {10}^{4} \frac{{nm}^{3}}{ICGJ NP} .$$

Furthermore, assuming that ICGJ@PEI NPs have a uniform spherical shape, the volume of PEI around a single ICGJ NP can be written as:

$${V}_{pei} = \frac{4}{3} π [ {( {R}_{icgj} + {R}_{pei} )}^{3} - {R}_{icgj}^{3} ] .$$

In the equation above, all variables, except for Rₚₑᵢ, are known (Vₚₑᵢ=1.43×10⁴ nm³, Rₚₑᵢ=25 nm). Therefore, Rₚₑᵢ can be found: Rₚₑᵢ=1.97 nm.

Required PEI to Form a Monolayer around the ICGJ NPs

The objective is to find the number of PEI_{1.8K} molecules needed to form a monolayer around one ICGJ NP. To find this number, the 2D representation of the PEI radius of gyration is needed. Based on the molecular weight of PEI, its 3D radius of gyration can be predicted from the following equation: Rₓ=0.0808M_{w}^{0.45}. For PEI with a MW of 1800, the predicted 3D radius of gyration is 2.36 nm, which is close to the reported value of 2.76 nm in literature for fully extended (when protanated) PEI with a MW of 2000.

PEI has a monomer weight of 43.04, and for PEI with MW=1800, the degree of polymerization (DPN) is ˜42. Therefore, a parameter “a”, which is defined as:

$$α = \frac{{R}_{g}}{{DPN}^{0.6}} ,$$

can be calculated. This parameter enables the calculation of the 2D radius of gyration from the 3D radius of gyration value. The 3D and 2D values are related by: R_{g(2D)}=a.DPN^{0.75}. By having the 2D radius of gyration, the 2D projection of a PEI molecule on ICGJ surface can easily be found by: A_{PEI}=πR_{g(2D)}². Based on the input parameters, each PEI_{1.8K} molecule occupies an area of ˜65 nm². The available surface area per ICGJ is found by: A_{ICGJ}=4πR_{ICGJ}². Thus, the number of close packed PEI molecules needed to cover the ICGJ surface is defined by:

$${\#}_{PEI , req} = \frac{{A}_{icgj}}{{A}_{PEI}} .$$

For a 50 nm ICGJ NP, ˜141 PEI molecules are needed to fully cover the ICGJ surface.

The second objective is to calculate whether enough PEI molecules are available to fully cover all ICGJ molecules. The number of ICGJ NPs available can be calculated from the input ICGJ mass (Mimi, feed), and the diameter of the ICGJ NPs (RICGJ):

$${V}_{icgj} = \frac{4}{3} π {R}_{icgj}^{3} and {\#}_{icgj} = \frac{{M}_{icgj , feed}}{{ρ}_{icgj} \times {V}_{icgj}} ,$$

where the density of ICGJ is 0.74 of the bulk ICG density (because the molecules are ordered in the HCP conformation in ICGJ). The average ICGJ NP diameter is 50 nm. The number of available PEI molecules can be calculated from the input mass of

$$PEI ( {M}_{pei , feed} ) {\#}_{PEI , avail .} = \frac{{M}_{PEI , Feed}}{{M}_{W}} \times {N}_{avogadro} .$$

Therefore, the ratio of available PEI molecules to ICGJ NPs can be easily calculated:

$${( \frac{PEI}{ICG} )}_{avail .} = \frac{{\#}_{PEI , avail}}{{\#}_{ICGJ}} .$$

Based on a PEI mass of 5.4 mg, this ratio is equal to ˜172. Therefore, the number of PEI molecules available is 1.22 times higher than the minimum number needed to form a monolayer around ICGJ NPs.

FIG. 10 shows the zeta potential of ICGJ@PEI NPs in three different cases. The “Optimized PEI” data series, which the calculations above are based on, shows that the zeta potential of ICGJ@PEI NPs are similar to that of PEI_{1.8k-branched}. This indicates that ICGJ NPs must be covered with at least a monolayer of PEI. For the case where the amount of added PEI was doubled, no significant change in the ICGJ@PEI NPs' zeta potential was observed. However, in the case where only half of the optimized amount was added, the zeta potential was significantly less positive for ICGJ@PEI NPs at pH values of 4 and 7. This indicates that not all ICGJ NPs are covered with a PEI layer.

### ICGJ@PEI Polymersomes

Calculation of Encapsulation Efficiency and Polymer Recovery

In order to determine the encapsulation efficiency of ICGJ as shown in the results section, the polymersome solution was first mixed with an equal volume of ethanol to drive the J aggregates back to ICG monomer. As shown in FIG. 2, UV-vis-NIR-NIR spectroscopy analysis demonstrated that after ˜30 minutes the peak for ICGJ is completely gone indicating complete dissociation of the aggregates. The amount of encapsulated ICG was then determined from the standard concentration-OD curve of ICG in FIG. 5 in a 50-50 water-ethanol mixture. To calculate the amount of PLGA-PEG recovered in the form of Polymersomes, after purification to remove free polymer, TOC analysis was used to determine the total C mass of the sample, CT, such that: Carbon mass of recovered PLGA-PEG (mg)=C_{T} (mg)−carbon mass of ICGJ in ICGJ@PEI (from concentration-OD curve, FIG. 5) (mg)−carbon mass of PEI (mg). The carbon mass of PEI was determined from the ratio of PEI to ICGJ, which was calculated separately by TOC before encapsulation.

Release of ICG from Polymersomes Determined by Dialysis

A separate set of samples was enclosed in dialysis bags (cellulose membrane, MW cut-off 3.5 kDa, Fisherbrand #21-152-9) and incubated in 40 mL of the same media (DI or FBS with the same pH) at 37° C. while stirring. The release of ICG was monitored with UV-vis-NIR spectra. After sampling, the tested solution was returned back to the incubation medium. In 24 hours, the amount of ICG released in DI water at pH 5.5 was ˜2% in DI water and ˜15% in FBS. A control experiment to determine the release of ICG from Polymersomes (instead of ICGJ@PEI) was also performed.

ICGJ@PEI Polymersome Formulation Optimization

TABLE 1
Summary of optimized experimental parameters for the
synthesis of ICGJ@PEI PEG-PLGA Ps and notable properties
based on these experimental parameters.
Optimized Input Experimental Parameters

| | |
|---|---|
| ICGJ@PEI concentration (mg/mL) | 50 |
| PEI M_{w}/type | 1800/branched |
| PEI concentration (wt %) | 10 |
| PEI volume added to ICGJ (μL) | 100 |
| W₁ volume (μL) | 500 |
| PEG-PLGA concentration (mg/mL) | 25 |
| O (chloroform) volume (mL) | 1 |
| ICGJ in W₂ phase concentmtion (mg/mL) | 5 |
| PVA concentration (mg/mL) | 50 |
| Dilution of W₂ phase in DI water (mL) | 40 |
| Sonication intensity (%) (both emulsions) | 30 |

Average Properties Based on Optimized Experimental Parameters
Including Standard Deviation (n = 6 forall experiments,
except n = 9 for I₈₉₀/I₇₈₅ and Ps D_{H})

| | |
|---|---|
| ICGJ@PEI hydrodynamic diameter [(nm) | 56 ± 5 |
| Polymersome I₈₉₀/I₇₈₅ | 1.6 ± 0.1 |
| Polymersome vol avg. hydrodynamic | 77 ± 8 |
| diameter D_{H} (nm) | |
| Encapsulation efficiency (%) | 27 ± 4 |
| Polymer recovery (%) | 39 ± 5% |
| ICG/Polymer (w/w) | 0.7 ± 0.1 |

Calculations to Describe the Nanocapsule Composition and Morphology

Measurement of PEG density on the surface of the polymersomes using ¹H NMR

¹H NMR was employed to determine both the total PEG content and the surface PEG content of the PEG-PLGA polymersomes using Xu et al. method (Xu Q et al., 2015, ACS Nano, 9:9217-9127). The total PEG content was determined by dissolving the polymersomes in deuterated chloroform (CDCl₃), while the surface PEG content was determined by re-dispersing the dried polymersomes in D₂O. The surface PEG shows a broadening peak at 3.6 ppm, which can be used for quantitative analysis of PEG surface density. The PEG content was determined by comparing to a PEG₁₀ₖ calibration curve calculated from ¹H NMR spectra.

The measurements were performed using a Bruker 400 REM at 400 MHz with a relaxation time of 10 seconds and angle of 90°. A known mass of PEG₁₀ₖ homopolymer in D₂O was serially diluted for generation of a calibration curve for the PEG signal in ¹H NMR, and this calibration curve was used to calculate the surface PEG content on polymersomes. A set amount (0.05 mg/mL) of 3-(trimethylsilyl)propionic acid-D₄ sodium salt was added to all samples as the reference. To determine the mass fraction of polymersomes in solution, 0.1 mL of polymersomes in D₂O solution was lyophilized and weighed. The surface PEG density was calculated as the number of PEG molecules per 100 nm² surface area of polymersomes.

The PEG density was calculated as the number of PEG chains per 100 nm² of the polymersomes surface area. The PEG density, 2Γ, represents the number of PEG molecules on the inner and outer polymersome surfaces. This number was obtained by dividing the total PEG content (MPEG, units in mole) from ¹H NMR by the total surface area of the nanoparticles:

$$2 Γ = \frac{{M}_{PEG} \times 6.02 \times {10}^{23}}{\frac{{W}_{NP}}{{d}_{NP}} / \frac{4}{3} π {r}^{3}} / 4 π {r}^{2} ,$$

where W_{NP} is the total mass of the nanoparticles, d_{NP} is the density of the polymersomes, which is assumed to be equal to the density of the polymer, and r is the radius of the polymersomes, determined from TEM and DLS.

The surface area occupied per PEG chain occupies an area at the interface given by a sphere of diameter ε. This diameter is given by: ε=0.76 m^{0.5}, where c is given in angstroms and m is the molecular weight of the PEG chain. Thus, PEG¬₁₀ₖ has an unconstrained molecule sphere with a diameter of 7.6 nm and occupies a surface area of 45.36 nm² (This surface area is given by π*(ε/2)²). According to Auguste (Auguste D T et al., 2006, Biomaterials, 27:2599-2608), full mushroom coverage, F*, represents the number of unconstrained PEG molecules per 100 nm². Therefore, for PEG^{10k}, Γ*=2.2 PEG chains. For a 65 nm polymersome, F was found to be 2.4. Based on these values, Γ/Γ*=1.1. Ratios with a value of lower than 1 indicate a low density and PEG molecules being in the mushroom conformation, while ratios with a value higher than 1 indicate a high density and PEG molecules in a brush conformation.

TABLE 2
Percentage of Surface PEG with Respect to the Total PEG
Present in the System.

| sample | PEG/TMS @ D₂O | PEG/TMS @ CDCl₃ | Surface PEG/ |
|---|---|---|---|
| # | (Surface PEG) | (Total PEG) | Total PEG (%) |
| 1 | 3.7 | 3.85 | 96% |
| 2 | 3.46 | 4.01 | 86% |
| 3 | 3.52 | 3.96 | 89% |

Calculation of ICGJ/ICG Ratio Inside the Polymersomes

To find the ICG concentration, in the monomer form, inside the polymersomes, the polymersomes' absorbance peak in ˜790 nm was recorded. The concentration of ICG was found from the calibration curve shown in FIG. 26. Next, the polymersomes were dissolved in 1/1 v/v ethanol/DI water solution to dissociate all the ICGJ NPs. The total ICG concentration (in monomer form+in J-aggregate form) was found from the calibration curve shown in FIG. 5.

Sample Calculation

1. A polymersome sample, diluted 100 times in DI water, yields an absorbance value of 0.801 @895 nm and 0.498 @790 nm. From the first calibration curve, an ICG concentration of 0.001642 mg/mL is obtained.

2. The sample is then diluted with an equal amount of ethanol (total: 200 times dilution). The absorbance value @790 nm is now 1.770. From the second calibration curve, this yields a concentration of 0.004464 mg/mL.

3. Accounting for the dilutions, the initial ICG concentration is 0.1642 mg/mL and the total ICG concentration is 0.8928 mg/mL. By subtracting the initial amount of monomeric ICG from the total amount, an ICGJ concentration of 0.7286 mg/mL is found.

4. Therefore, the weight ratio of ICGJ to ICG in polymersome is equal to 0.7286/0.1642=4.43.

FIG. 9 shows UV-vis-NIR spectra of ICGJ@PEI polymersomes compared to a 83/17 wt % ICGJ/ICG mixture. The similar intensities of the peaks attributed to ICGJ and ICG at 895 nm and 795 nm are in agreement with the calculated weight ratio of 4.4.

Polymersome Loading, Encapsulation Efficiency, and Polymer Recovery Calculations

Calculation of Total ICG (Monomer and J Form) in the Polymersome Sample

The concentration of total ICG in the Polymersome system (CI) is found by diluting the Polymersomes in a 1/1 mixture of ethanol/water and comparing the absorbance value to a calibration curve of ICG in 50/50 mixture of ethanol/water. From this concentration, the mass of ICG (both in monomer and J form) is calculated.

An aliquot of the polymersome sample is taken (V_{UV-1}) and diluted to a volume of 1.5 mL (V_{UV-2}). UV-vis-NIR is run on this sample. (For example, 0.01 mL of polymersome sample dispersed in DI water is diluted with 1.5 mL DI water and ethanol). The dilution factor for the UV-vis-NIR sample is defined as the ratio of the diluted volume to the aliquot taken from the polymersome sample (For example, for an aliquot of 0.01 mL and total volume of 1.5 mL the dilution factor is 150.)

$${DF}_{UV} = \frac{{V}_{UV - 2}}{{V}_{UV - 1}} .$$

The absorbance of the UV-vis-NIR sample at 790 is recorded (Ab_{I}). From the 1/1 ethanol/water calibration curve (FIG. 27), and dilution factor, the concentration of the ICG (both in monomer and aggregate form) in aliquot can be found:

$${C}_{I} = \frac{{Ab}_{I x} {DF}_{UV}}{396.49} .$$

(For example, for the sample above, the absorbance is ˜1.5. For a dilution factor of 150, the aliquot concentration is 0.57 mg/mL). Therefore, the total ICG mass (both in monomer and J form) in the polymersome sample is found by multiplying the concentration by total polymersome sample volume: M_{I}=(C_{I})(Vₛₐₘₚₗₑ).

Calculation of ICG in the Monomer Form in Polymersome Sample Determined by Dilution in DI Water

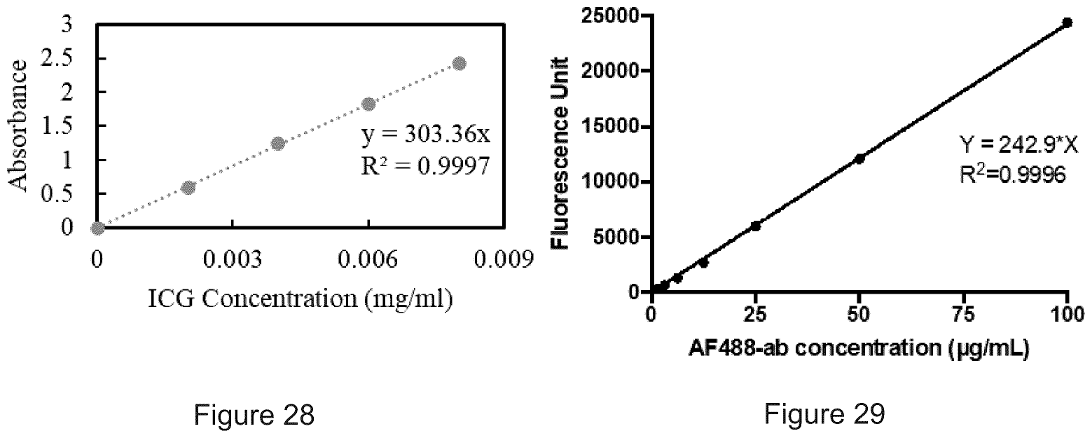

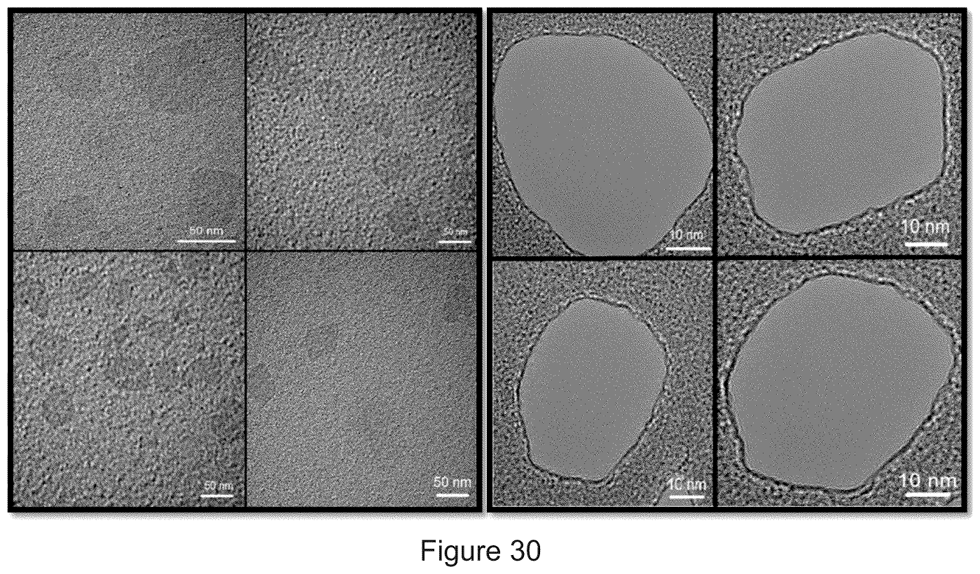

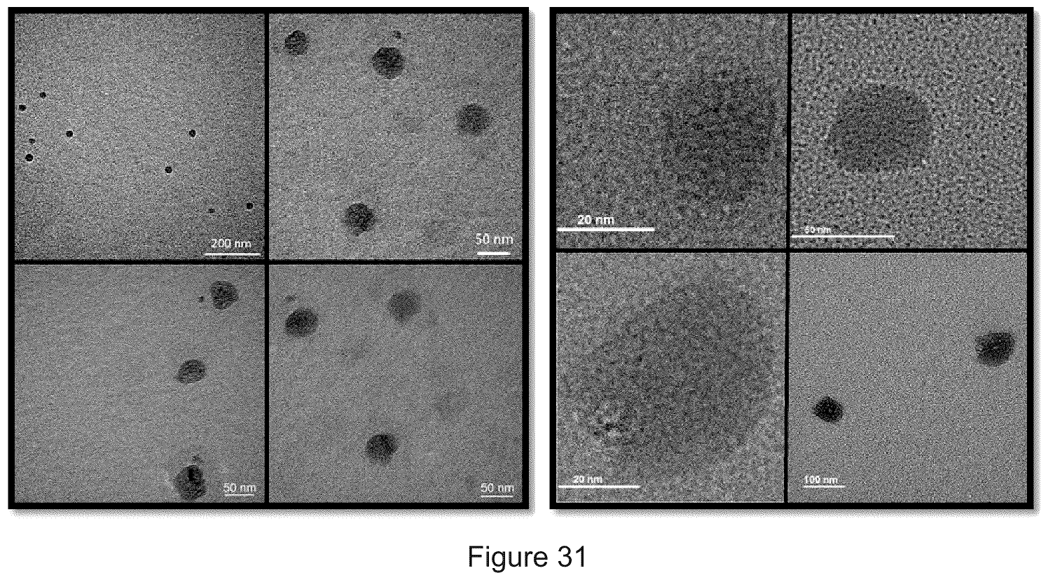

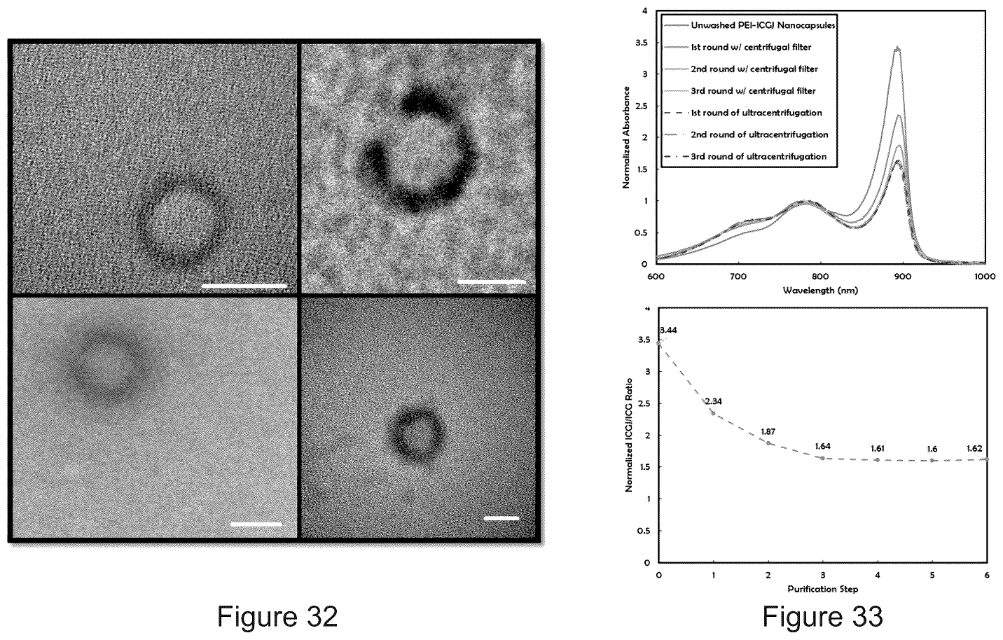

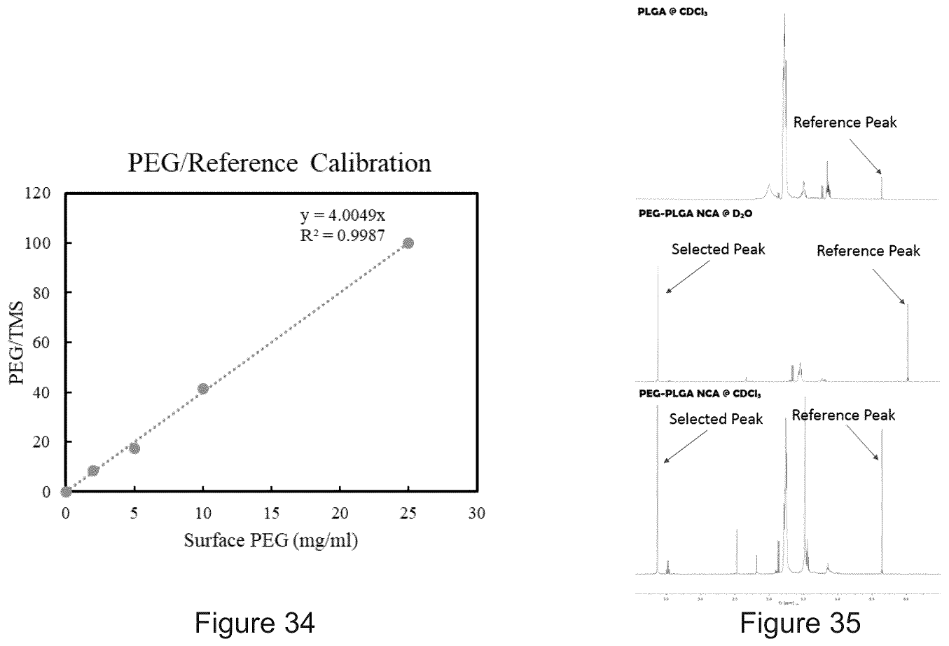

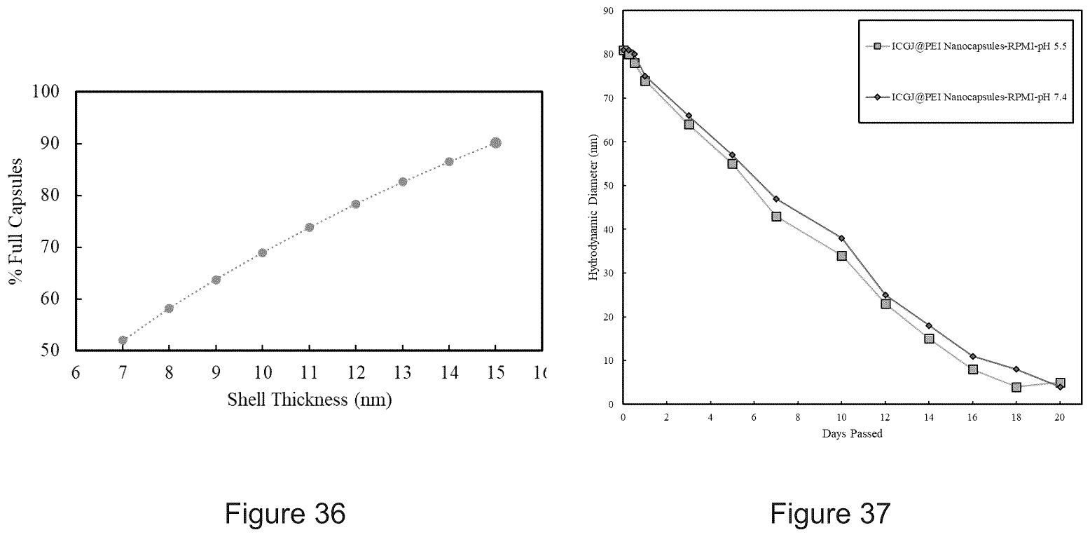

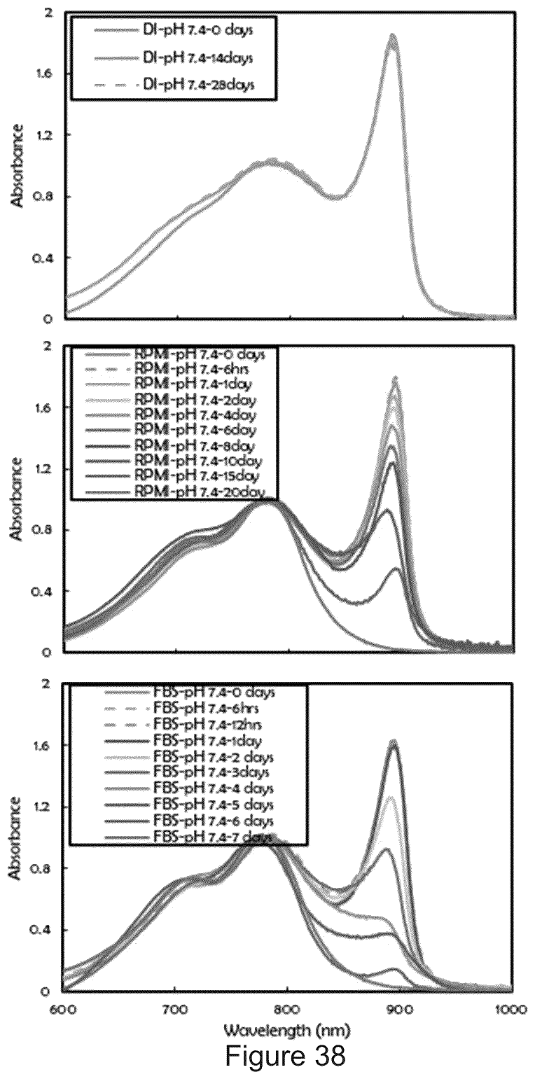

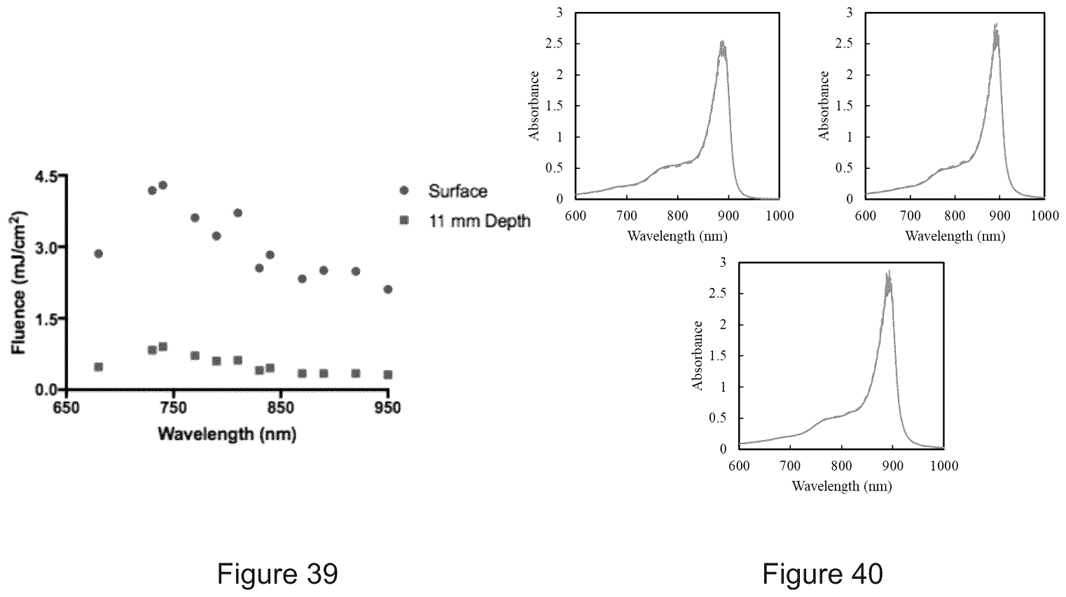

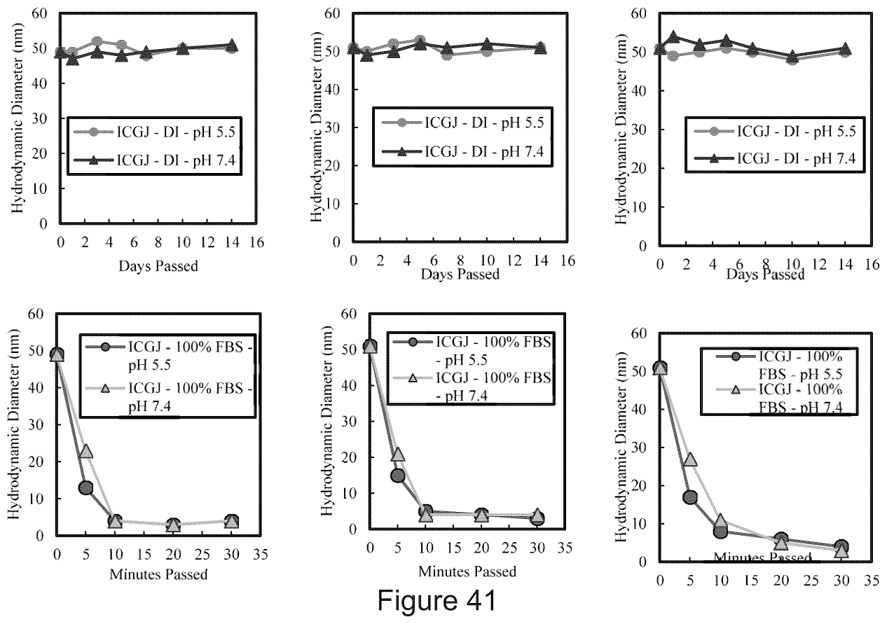

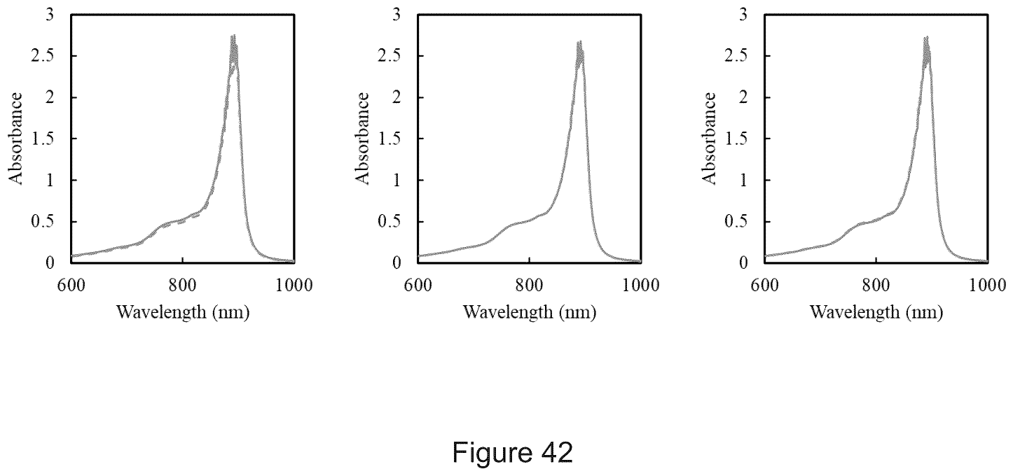

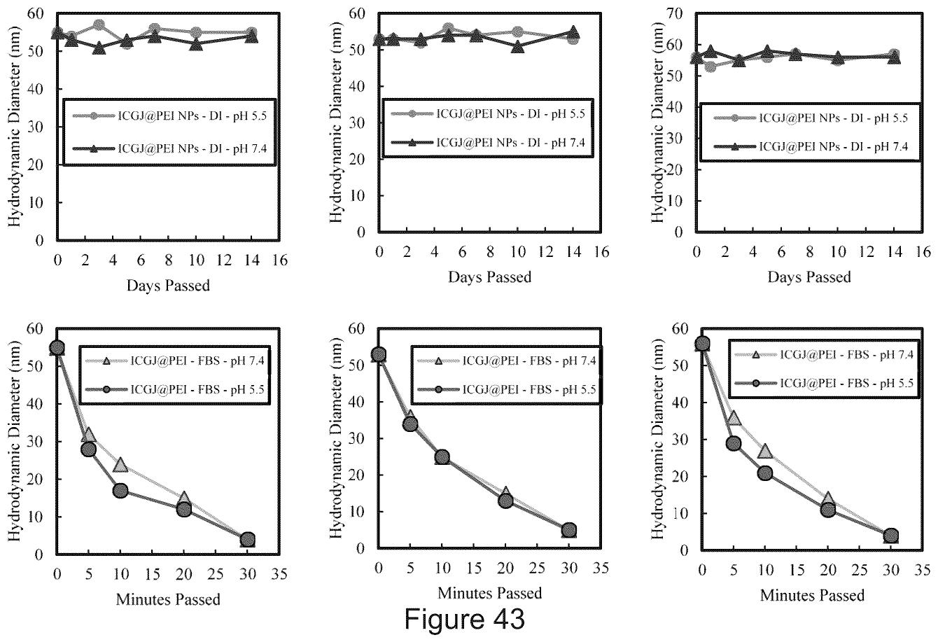

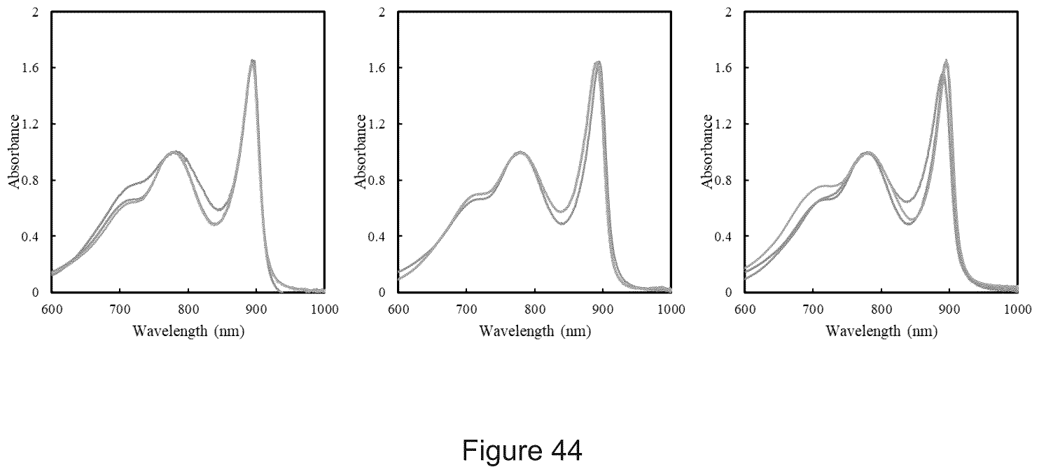

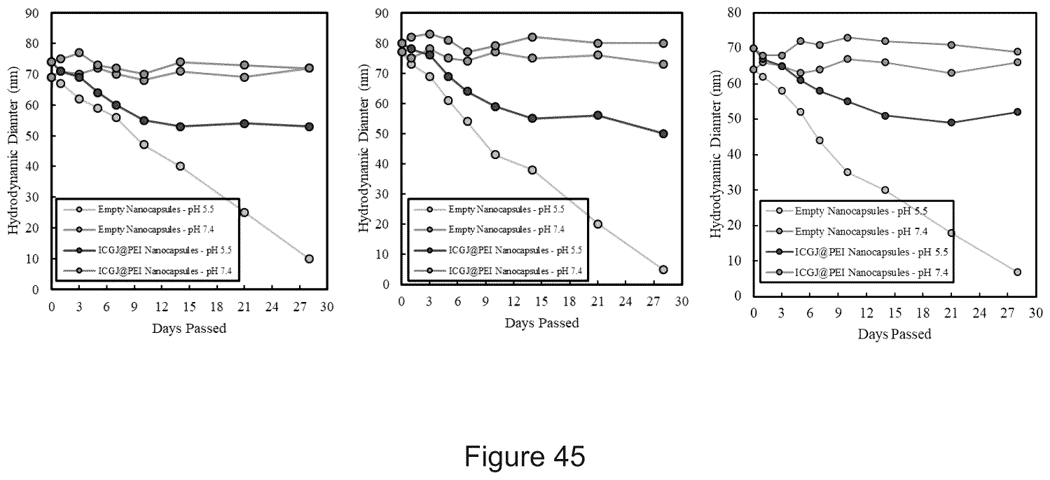

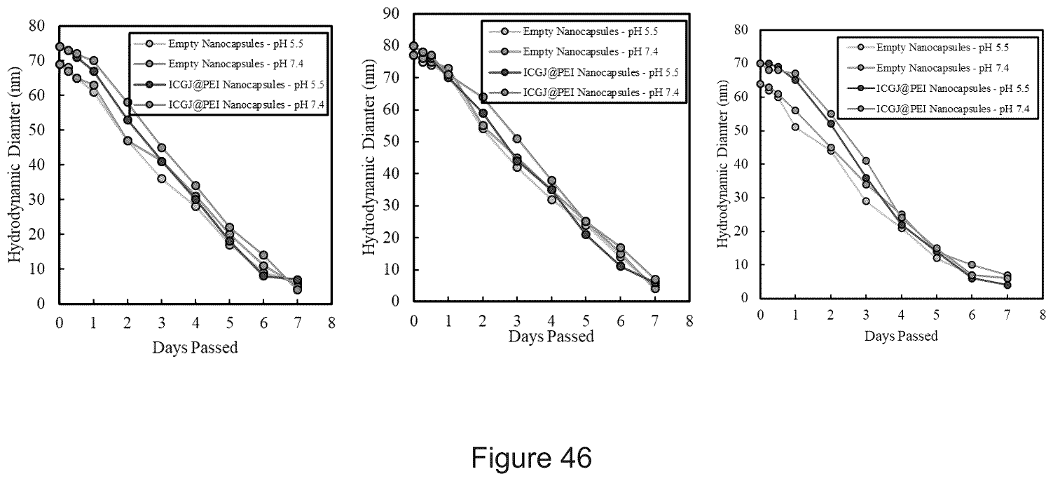

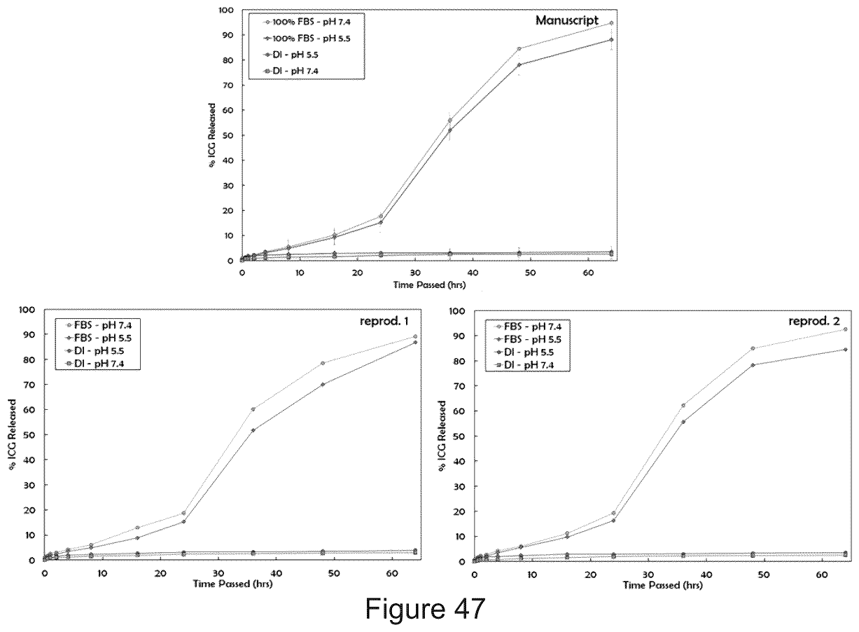

The concentration of ICG monomers in the sample was found by recording the absorbance value of the Polymersomes diluted in DI water at 790 nm wavelength and comparing the value to a calibration curve of ICG dissolved in DI water (FIG. 28).

An aliquot of the polymersome sample is taken (V_{UV-1}) and diluted to a volume of 1.5 mL (V_{UV-2}). UV-vis-NIR is run on this sample. (For example, 0.01 mL of polymersome sample dispersed in DI water is diluted with 1.5 mL DI water). The dilution factor for the UV-vis-NIR sample is defined as the ratio of the diluted volume to the aliquot taken from the polymersome sample (For example, for an aliquot of 0.01 mL and total volume of 1.5 mL the dilution factor is 150.)

$${DF}_{UV} = \frac{{V}_{UV - 2}}{{V}_{UV - 1}} .$$

The absorbance of the UV-vis-NIR sample at 790 is recorded (AbI). From the 1/1 Ethanol/water calibration curve, and dilution factor, the concentration of the ICG (both in monomer and aggregate form) in aliquot can be found:

$${C}_{I °} = \frac{{Ab}_{I ° x} {DF}_{UV}}{303.36} \cdot M {°}_{I} = ( C {°}_{I} ) ( {V}_{sample} ) .$$

Therefore, the total mass of ICGJ (MU) in the polymersome sample can be found by: M_{IJ}=M_{I}−M º_{I}.

Calculation of Polymer Recovery and Loading from TOC

An aliquot of the polymersome sample (dispersed in DI water) (V_{TOC-1}) is taken and diluted with DI water (V_{TOC-2}). (For example, 0.5 mL of polymersome sample diluted to a total volume of 20 mL.) The dilution factor of the TOC sample (DF_{TOC}) is defined as:

$${DF}_{TOC} = \frac{{V}_{TOC - 2}}{{V}_{TOC - 1}} .$$

The TOC machine gives the concentration of the dilute sample in units of ppm (TOC_{raw}). To find the total carbon in the aliquot in mg, TOC was found, where: Mₜₒₜₐₗ^{C}=TOC_{raw}×V_{TOC-2}×10⁻³ mg per microgram.

First, the carbon contribution of ICG must be subtracted from the total carbon. Carbon contribution of ICG (monomer and J form) to the total carbon from TOC is: M_{I}^{C}=V_{TOC-}×C_{I}×CF_{I}. Carbon fraction of ICG (CFI) is 0.667 from its formula (C₄₃H₄₇N₂NaO₆S₂). Mass of remaining PEI in the sample is recorded separately by TOC. The carbon fraction of PEI (CFPEI) is 0.556 (C₂H₅N), therefore the carbon mass of PEI is found by: M_{PEI}^{C}=V_{TOC-1}×C_{PEI}×CF_{PEI}. Carbon mass of polymer is found from subtracting the PEI carbon mass and ICG carbon mass from the total carbon mass: M^{C}_{P}=(M^{C}ₜₒₜₐₗ)−(M^{C}_{PEI})−M^{C}_{ICG}). By knowing the carbon fraction of the PEG-PLGA (CFP=0.442), the concentration of the polymer in the aliquot can be found:

$${C}_{P} = \frac{{M}_{P}^{C} / {CF}_{P}}{{V}_{TOC - 1}} .$$

Multiplying the polymer concentration by total volume of the sample yields the mass of polymer: M_{P}=C_{P}×Vₛₐₘₚₗₑ, the mass of a single ICGJ NP is calculated by: M^{I}_{IJ}: (V_{IJ}¹(ρ_{IJ}) and

$${{M}^{1}}_{IJ :} ( \frac{4}{3} π {r}_{1}^{3} ) ( {ρ}_{IJ} ) .$$

Therefore, the number of ICGJ NPs in the polymersome sample is:

$${N}_{IJ} = \frac{{M}_{IJ}}{{M}_{IJ}^{1}} .$$

The volume of a single PEG-PLGA shell is found by:

$${{V}^{1}}_{S} = \frac{4}{3} π ( {r}_{2}^{3} - {r}_{1}^{3} ) .$$

In this equation, it is assumed that the polymer shell is tightly wrapped around the ICGJ and there is no free space between the ICGJ NPs and the polymer shell. From this volume, the total number of capsules can be calculated:

$${N}_{C} = \frac{{M}_{P}}{{ρ}_{P} \times {V}_{S}^{1}} ,$$

where the MP is obtained from TOC and ρ_{P} is 1340 mg/mL. The following variables can be calculated now: Encapsulation Efficiency

$$( EE \% ) = \frac{{M}_{I}}{{M}_{I , feed}} \times 100 ; Polymer Recovery ( \% ) = \frac{{M}_{P}}{{M}_{P , feed}} \times 100 ; \\ and Loading ( \% ) = \frac{{M}_{I}}{{M}_{P}} \times 100.$$

Calculation of Full (ICGJ Containing)/Empty Polymersomes

The volume of ICGJ NPs is calculated by:

$${V}_{icgj} = \frac{4}{3} π {R}_{icgj}^{3} .$$

From the concentration of ICG (C_{icgj}) (mg/mL) and the density of ICGJ, the number of ICGJ@PEI NPs in the sample can be calculated (assumption: these ICGJ NPs are covered with PEI):

$${\#}_{icgj} = \frac{{C}_{icgj}}{{ρ}_{icgj} \times {V}_{icgj}} .$$

Given the average polymersome diameter from DLS, the volume of one polymersome can be calculated:

$${V}_{P} = \frac{4}{3} {π ( {R}_{O} )}^{3} .$$

The volume of the polymeric shell is then obtained by:

$${V}_{PEG - PLGA} = \frac{4}{3} π [ {( {R}_{O} )}^{3} - {( {R}_{I} )}^{3} ] = \frac{4}{3} π [ {( {R}_{O} )}^{3} - {( {R}_{O} - {R}_{PEG - PLGA} )}^{3} ] .$$

Similar to ICGJ NPs, the number of polymeric shells (assuming all polymer chains have formed capsules can be calculated by:

$${\#}_{P} = \frac{{C}_{PEG - PLGA}}{{ρ}_{PEG - PLGA} \times {V}_{PEG - PLGA}} .$$

The number of ICGJ@PEI NPs (#_{icgj}) to number of polymer shells (#_{P}) can now be calculated. Assuming that each shell can only host ICGJ@PEI NP, the number of empty and full capsules can be calculated as well.

Calculation of Number of ICG Molecules Required to Occupy the Outer Water Surface of W/O/W Droplets at t=0

At t=0, the outer diameter of the PEG-PLGA W/O/W droplets is 460 nm by DLS right after the emulsion is made. This gives a volume of:

$${V}_{oil drop} = \frac{4}{3} {π ( \frac{460 nm}{2} )}^{3} = 5.09 \times {10}^{7} {nm}^{3} .$$

Assuming that all chloroform (1 mL) is converted into droplets containing small water droplets, the number of droplets for 1.5 mL W1/O emulsion is:

$${n}_{oil droplets} = \frac{{V}_{o i l} + {V}_{w a t e r}}{{V}_{oil drop}} = \frac{1.5 \times {10}^{21} {nm}^{3}}{5.09 \times {10}^{7} {nm}^{3}} = 2.95 \times {10}^{13} droplets .$$

On the other hand, it is known that ICG molecules have a footprint of 2.6 nm². Given that the area of the outer oil droplet:

$${A}_{outer} = 4 {π ( \frac{460 nm}{2} )}^{2} = 6.64 \times {10}^{5} {nm}^{2}$$

and the number of droplets are known, the total outer surface area and the number of ICG molecules needed to cover them can be calculated. A_{outer,total}=Aₒᵤₜₑᵣ×n_{oil droplets}=6.64×10⁵ nm²×2.94×10¹³=1.95×10¹⁹ nm². Therefore:

$${n}_{ICG , required} = \frac{{A}_{outer , total}}{{ICG}_{footprint}} = \frac{1.95 \times {10}^{19} {nm}^{2}}{2.6 {nm}^{2}} = 7.58 \times {10}^{18} ICG molecules .$$

The number of ICG molecules available in the outer water phase is:

$${n}_{ICG , available} = ( 4 ml \times 5 \frac{mg}{ml} ) \times ( \frac{1 {0}^{- 3} mol}{775 mg} ) \times ( \frac{6 \text{.022} \times {10}^{23}}{1 mol} ) = 1.55 \times {10}^{19} ICG molecules .$$

### Therefore:

$$\frac{{n}_{ICG , available}}{{n}_{ICG , required}} = \frac{1.55 \times {10}^{19}}{7.58 \times {10}^{18}} = 2.06 .$$

Now, the number of ICG molecules required to occupy the inner interface can be calculated. The inner water area of a single droplet is:

$${Λ}_{inner} = 4 {π ( \frac{50}{2} nm )}^{2} = 7850 {nm}^{2}$$

and the volume of a single water droplet is:

$${V}_{inner} = \frac{4}{3} {π ( \frac{50}{2} nm )}^{3} = 6.54 \times {10}^{4} {nm}^{3} .$$

Assuming that all the water in the W1 phase (0.5 mL) is converted in water droplets embedded in the oil droplet, the total number of water droplet is:

$${n}_{{H}_{2} O} = \frac{{V}_{{H}_{2} O tot}}{{V}_{i n n e r}} = \frac{5 \times {10}^{20} {nm}^{3}}{6.54 \times {10}^{4} {nm}^{3}} = 7.65 \times {10}^{15} .$$

Based on this, the number of water droplets embedded in the oil drop is:

$${n}_{\frac{{H}_{2} O d r o p l e t s}{oil drop}} = \frac{{n}_{{H}_{2} O d r o p l e t s}}{{n}_{oil droplets}} = \frac{7.65 \times {10}^{15} {nm}^{3}}{2.95 \times {10}^{13} {nm}^{3}} = 259$$

and the total inner surface area is: A_{inner,total}=Aᵢₙₙₑᵣ×n_{droplets}=7850 nm²×7.65×10¹⁵=6.05×10¹⁹ nm². The total amount of ICG molecules available in the inner water phase is:

$${n}_{ICG , available} = ( 0.5 ml \times 50 \frac{mg}{ml} ) \times ( \frac{1 {0}^{- 3} mol}{775 mg} ) \times ( \frac{6 \text{.022} \times {10}^{23}}{1 mol} ) = 1.94 \times {10}^{19} ;$$

$${n}_{required} = \frac{{A}_{i n ner , total}}{{ICG}_{f o o t p r i n t}} \frac{6.05 \times {10}^{19} {nm}^{2}}{2.6 {nm}^{2}} = 2.33 \times {10}^{19} ;$$

$$and \frac{{n}_{available}}{{n}_{r e q u i r e d}} = \frac{1.94 \times {10}^{19}}{2.32 \times {10}^{19}} = 0.8 3.$$

Therefore, the theoretical ICG concentration required to saturate the surface of 50 nm water droplets is:

$${C}_{ICG} = 50 \frac{mg}{ml} * \frac{1}{0.83} = 60 \frac{mg}{ml} .$$

### Characteristic of ICGJ@PEI Ps-Ab and Cellular Response in Photoacoustic Imaging Determining the Number of Conjugated Antibodies Per Polymersome

Anti-EGFR (clone C225) antibodies were labeled with Alexa Fluor 488 (AF488) following the manufacturer's instruction (Invitrogen). The labeled antibodies were then conjugated to ICGJ@PEI Ps. At least four samples were prepared. First, fluorescence of antibodies at the same concentration as in the conjugation reaction was measured. Then, fluorescence of all supernatants from washing of ICGJ@PEI Ps-Ab following the antibody's conjugation was determined. Finally, fluorescence of conjugated antibodies was calculated as follows: F_{ICGJ@PEI Ps-Ab}=F_{AF488-Ab}−Fₛᵤₚₑᵣₙₐₜₐₙₜₛ, where F_{ICGJ@PEI NC-Ab} is residual fluorescence that corresponds to the total amount of conjugated antibodies; FAF488-Ab is fluorescence of AF488 labeled anti-EGFR antibodies at the concentration used for conjugation; and Fₛᵤₚₑᵣₙₐₜₐₙₜₛ is fluorescence of all supernatants collected during washing of ICGJ@PEI Ps-Ab conjugates from excess of antibodies.

F_{ICGJ@PEI NC-Ab} was then used to calculate the number of antibodies bound to ICGJ@PEI Ps-Ab using a standard curve for AF488 labeled anti-EGFR antibodies (FIG. 29) and the number of ICGJ@PEI Ps. The number of polymersomes was calculated using the known concentration of PEG-PLGA polymer that was determined from the method described in the “Polymersome Loading, Encapsulation Efficiency, and Polymer Recovery Calculations” section:

$$ICGJ @ PEI Ps ( \frac{\#}{mL} ) = \frac{{C}_{polymer}}{( {ρ}_{PEG - PLGA} \times {V}_{ICGJ @ PEI Ps} )} ,$$

where C_{polymer} is concentration of PEG-PLGA in the ICGJ@PEI Ps (mg/mL); pPEG-PLGA is density of PEG-PLGA (mg/nm³) (Dancy J G et al., 2016, J. Control Release, 238:139-142); VICGJ@PEI Ps is Volume of ICGJ@PEI Ps (nm³)=Outer volume of ICGJ@PEI Ps—Inner volume of ICGJ@PEI Ps. Then, the number of antibodies bound to ICGJ@PEI Ps-Ab was divided by the number of ICG@PEI Ps resulting in ˜11±1 antibodies per ICGJ@PEI Ps-Ab.

### Additional Reproducibility Data

TABLE 3
DLS volume distributions of ICGJ NPs. The correlation function was
fitted with NNLS method; the distribution for the first population
of the correlation function fit is shown for six samples.

| Diameter (nm) | Sample 1 | Sample 2 | Sample 3 | Sample 4 | Sample 5 | Sample 6 |
|---|---|---|---|---|---|---|
| 35 | 4 | 0 | 17 | 0 | 0 | 0 |
| 40 | 18 | 19 | 24 | 0 | 21 | 0 |
| 45 | 55 | 63 | 27 | 34 | 31 | 59 |
| 50 | 14 | 13 | 11 | 29 | 28 | 27 |
| 55 | 6 | 1 | 8 | 22 | 10 | 0 |
| 60 | 0 | 0 | 0 | 2 | 0 | 0 |
| Average (nm) | 45 | 44.8 | 44.2 | 50.7 | 49.2 | 47.1 |
| Std. Dev. (nm) | 4.5 | 5.4 | 2.1 | 3.7 | 3.1 | 5.5 |

TABLE 4
Zeta potential of ICGJ NPs. The values and error bars shown in
FIG. 3 are based on this table.

| pH | 4 | 7 | 10 |
|---|---|---|---|
| Sample 1 | −42.1 | −48.41 | −59.49 |
| Sample 2 | −40.8 | −45.93 | −48.12 |
| Sample 3 | −32.0 | −41.12 | −55.9 |
| Average (mV) | −38.3 | −45.1 | −54.5 |
| Std. Dev. (mV) | 5.5 | 3.7 | 5.8 |

TABLE 5
DLS volume distributions of ICGJ@PEI NPs. The
correlation function was fitted with the NNLS method.

| Diameter (nm) | Sample 1 | Sample 2 | Sample 3 | Sample 4 | Sample 5 | Sample 6 |
|---|---|---|---|---|---|---|
| 35 | 0 | 0 | 0 | 0 | 0 | 0 |
| 40 | 0 | 0 | 0 | 18 | 9 | 0 |
| 45 | 9 | 0 | 0 | 29 | 24 | 0 |
| 50 | 55 | 34 | 19 | 31 | 28 | 15 |
| 55 | 22 | 31 | 25 | 16 | 19 | 26 |
| 60 | 8 | 22 | 24 | 3 | 7 | 27 |
| 65 | 0 | 0 | 11 | 0 | 0 | 14 |
| 70 | 0 | 0 | 5 | 0 | 0 | 2 |
| Average (nm) | 51.5 | 54.3 | 57.5 | 48.2 | 49.4 | 57.0 |
| Std. Dev. (nm) | 5.2 | 6.2 | 6.0 | 6.0 | 5.3 | 6.6 |

TABLE 6
Zeta potential of ICGJ@PEI NPs. This table provides the values
and error bars shown in FIG. 3A.

| pH | 4 | 7 | 10 |
|---|---|---|---|
| Sample 1 | 35.66 | 26.78 | −2.77 |
| Sample 2 | 37.12 | 27.33 | −3.24 |
| Sample 3 | 32.15 | 23.55 | −10.53 |
| Average (mV) | 35.0 | 25.9 | −5.5 |
| Std. Dev. (mV) | 2.6 | 2.0 | 4.3 |

TABLE 7
Zeta potential of ICGJ@PEI Ps. The values and error bars shown in
the manuscript are based on the following:

| pH | 4 | 7 | 10 |
|---|---|---|---|
| Sample 1 | −0.59 | −3.68 | −10.02 |
| Sample 2 | −1.14 | −5.55 | −9.51 |
| Sample 3 | −6.44 | −9.08 | −9.08 |
| Average (mV) | −2.7 | −6.1 | −9.5 |
| Std. Dev. (mV) | 3.2 | 2.7 | 0.5 |

TABLE 8
Zeta potential of ICGJ@PEI P-Abs. The values and error bars shown
in the manuscript are based on the following:

| pH | 4 | 7 | 10 |
|---|---|---|---|
| Sample 1 | −0.53 | −6.95 | −13.11 |
| Sample 2 | −2.78 | −1.39 | −8.05 |
| Average (mV) | −1.7 | −4.2 | −10.6 |
| Std. Dev. (mV) | 1.6 | 3.9 | 3.6 |

TABLE 9
DLS volume distributions of optimized ICGJ@PEI Ps. The correlation
function was fitted with NNLS method; the distribution for the first population
of the correlation function fit can be seen below for nine samples:

| Diameter | Sample | Sample | Sample | Sample | Sample | Sample | Sample | Sample | Sample |
|---|---|---|---|---|---|---|---|---|---|
| (nm) | 1 | 2 | 3 | 4 | 5 | 6 | 7 | 8 | 9 |
| 60 | 0 | 0 | 0 | 0 | 5 | 8 | 0 | 11 | 0 |
| 65 | 12 | 14 | 8 | 11 | 8 | 21 | 0 | 31 | 0 |
| 70 | 22 | 29 | 9 | 21 | 11 | 26 | 5 | 35 | 4 |
| 75 | 25 | 29 | 10 | 29 | 12 | 18 | 16 | 14 | 15 |
| 80 | 14 | 15 | 14 | 18 | 14 | 7 | 27 | 0 | 25 |
| 85 | 4 | 2 | 16 | 9 | 14 | 1 | 26 | 0 | 27 |
| 90 | 0 | 0 | 13 | 0 | 12 | 0 | 15 | 0 | 16 |
| 95 | 0 | 0 | 8 | 0 | 9 | 0 | 4 | 0 | 7 |
| 100 | 0 | 0 | 7 | 0 | 7 | 0 | 0 | 0 | 0 |
| 105 | 0 | 0 | 6 | 0 | 5 | 0 | 0 | 0 | 0 |
| 110 | 0 | 0 | 5 | 0 | 2 | 0 | 0 | 0 | 0 |
| 115 | 0 | 0 | 0 | 0 | 1 | 0 | 0 | 0 | 0 |
| Average | 73.4 | 72.8 | 92.0 | 74.6 | 82.9 | 69.8 | 76.5 | 67.8 | 83.0 |
| (nm) | | | | | | | | | |
| Std. Dev. | 6.8 | 8.3 | 4.1 | 7.6 | 3.5 | 6.6 | 8.6 | 10.8 | 10.1 |
| (nm) | | | | | | | | | |

TABLE 10
Encapsulation efficiency, polymer recovery, and loading for six
ICGJ@PEI Ps based on optimized conditions.

| Sample | EE (%) | PLM Recovery (%) | Loading (ICG/PLM) |
|---|---|---|---|
| Sample 1 | 23.2 | 37.8 | 0.61 |
| Sample 2 | 20.6 | 39.0 | 0.53 |
| Sample 3 | 25.9 | 35.6 | 0.73 |
| Sample 4 | 31.3 | 42.0 | 0.75 |
| Sample 5 | 31.3 | 45.0 | 0.70 |
| Sample 6 | 27.0 | 32.0 | 0.84 |
| Average | 26.6 | 38.6 | 0.7 |
| Std. Dev. | 4.3 | 4.6 | 0.1 |

### Example 2: Method to Make ICG@PEI Loaded Polymersomes with an Extinction Intensity Ratio at 895 nm Versus 790 nm (I₈₉₅/I₇₉₀) Ratio of 1.6—Standard Conditions

Formation of ICG J aggregates. 1.5 mM (1.15 mg/mL) ICG aqueous solution was heated in an oil bath at 65° C. while stirred at 750 rpm using a rod-shaped magnetic stir bar for 24 hours and the formation of ICGJ was monitored with DLS and UV-vis-NIR-NIR spectroscopy. After 24 hours, 6 mL ICGJ solution was sonicated with ultra-sonication probe for 1 min/mL at 15% output power (model: Branson™ S-450 Digital Sonifier, probe length: 16 cm, tip diameter: cm) and purified by filtration through a 0.22 μm syringe filter to remove any possible large aggregates. It was then dialyzed (cellulose membrane, MW cut-off 3 kDa, Sigma) against DI water for 18 hours. The DI water is refreshed every 6 hours to permeate any ICG monomers. To achieve the desired concentration of ICGJ, the solution was centrifuged with 30 kDa MWCO centrifugal filters at 5000 rpm for ˜2 hours.

Coating of ICG J aggregates with PEI. To coat the anionic ICGJ nanoparticles with a cationic PEI shell, 100 μL of 10 wt % PEI solution was added to ˜0.5 mL solution of ˜50 mg/mL ICGJ and the pH was set to 4 with dropwise addition of 1 N HCl to nearly fully protonate the PEI. The mixture was sonicated with a bath-sonicator (model: Fisherbrand™ CPXH series model 2800) for 5 min. and further stirred at room temperature for 2 hours. After forming ICGJ@PEI, the solution was mixed with 0.1 mL ethanol for 2 minutes to fully dissociate any uncoated ICGJ aggregates. Finally, the solution was washed and centrifuged with kDa MWCO centrifugal filters (regenerated cellulose membrane) with 15 mL capacity at 5000 rpm 3 times with 10 mL DI water to remove the free ICG and PEI chains in the solution.

Formation of Polymersomes with Encapsulated ICGJ@PEI

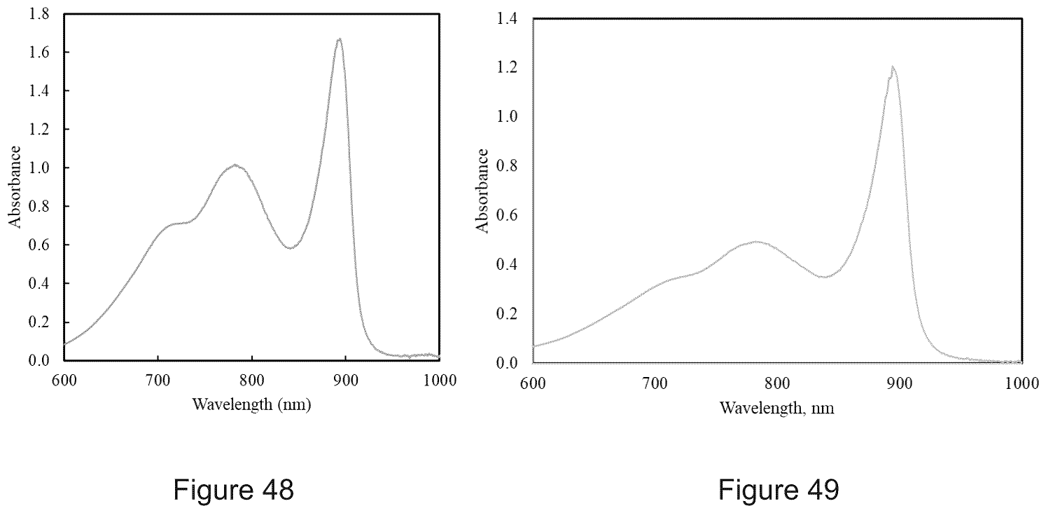

Azide-PEG-PLGA polymersomes were prepared using a double emulsion (W/O/W emulsion) process. The oil phase was prepared by dissolving A-PEG-PLGA (25 mg, 100 k 75:25 L/G) in chloroform (1 mL). The inner water phase was 500 μL of −50 mg/mL ICGJ@PEI solution. The first emulsion (W/O) was formed under sonication using a Branson™ S-450 Digital Sonifier probe sonicator for 6 times (10 seconds each, amplitude 30%). To generate a double emulsion (W/O/W), the W/O emulsion was added within 2-3 seconds to 4 mL aqueous solution of 5 mg/mL ICGJ (to reduce the dissociation of ICGJ@PEI in the inner water phase) and PVA (5 wt %, average MW 18000), followed by sonication for 6 times (10 seconds each, amplitude 30% gaps of 5-10 seconds for cooling). The rapid mixing was needed since the water droplets in the W/O emulsion settled in a period of about 1 minute. Afterwards, the double emulsion was diluted in 40 mL DI water and stirred magnetically with a rod-shaped stir bar at room temperature overnight to evaporate the chloroform and form the polymersomes. The sample was incubated for 10 minutes with 10% v/v ethanol/water solution and then centrifuged three times using Amicon Ultra-15 30 kDa MWCO centrifugal filters with 15 mL capacity (regenerated cellulose membrane, Millipore Sigma) at 5000 rpm for 15 minutes each to remove the non-encapsulated ICGJ@PEIs from the solution (5 mL aliquots of the polymersome sample were mixed with 8.5 mL of DI water and 1.5 mL of ethanol, making a 10% ethanol solution). During completion of the third round of diafiltration with the centrifugal filter, the 5 mL Ps aliquot was concentrated to a volume of (1.5 mL). This dispersion was purified by centrifugation at 30000 rpm for 30 minutes and rinsing with DI water 3 times. After each round of ultracentrifugation, the formed pellet containing Ps was re-dispersed in DI water to its original volume (1.5 mL) and then probe sonicated (with an intensity of 15%) for 10 seconds. The UV-vis-NIR spectrum of the final polymersome after purification is shown in FIG. 48.

### Example 3: Method to Make ICG@PEI Loaded Polymersomes with an Extinction Intensity Ratio I₈₉₅/I₇₉₀ Ratio>1.6 in this Case 2.4

The preparation of the ICGJ and the ICGJ@PEI is the same as described in example 2. To form the polymersomes, the W1 phase was 500 μL of ICGJ@PEI solution with a concentration of 50 mg/mL; oil phase was 25 mg Azide-PEG-PLGA (100 k 90:10 L/G) in 1 mL chloroform. To make the first W₁/O emulsion, the W1 and oil phases were mixed and probe sonicated using a Branson™ S-450 Digital Sonifier for 10 seconds with 30% amplitude. A concentrated stock solution of PVA was prepared ahead of time (133.3 mg/mL in water; heating to 65° C. until complete dissolution of PVA in about 3 h). The outer W2 solution was prepared as follows: probe sonicate 3.2 mL ICGJ in a 15 mL tube (3 min, 15% intensity) and then make a solution 5 mg/mL ICGJ with water and PVA (5 wt %, MW 13000-23000). This procedure requires 4 mL for one Ps batch. After sonication of ICGJ solution, 2.81 mL of ICGJ solution was mixed with 1.69 mL of PVA (133.3 mg/mL). The W₂ solution contained ICGJ to serve as a “sacrificial” surfactant to reduce the dissociation of ICGJ@PEI in the inner W₁ water phase. Before adding the first emulsion to the W2 solution, the sonication probe was moved to the center of the final solution to allow formation of a homogeneous emulsion. Two people performed this step to move the probe and add the first emulsion to 4 mL of W2 solution in 2-3 seconds. The mixture was probe sonicated twice (10 seconds each time with 30% amplitude) to make the W₁/O/W₂ emulsion. The double emulsion was immediately diluted in 40 mL DI water and stirred with a rod-shaped stir bar at 1200 rpm at room temperature for two days to evaporate the chloroform and form the polymersomes.

Centrifugation to Remove PEG-PLGA Residue not in Polymersomes

4 mL of polymersomes were diluted with 9.5 mL DI water in a 15 mL plastic tube and centrifuged 3 minutes at 5000 rpm to remove excess PEG-PLGA (deposited at the bottom of the tube).

Purification with Ethanol/Water Mixture to Dissociate Unencapsulated ICGJ

The supernatant from the previous step was carefully transferred into a 30K or 100K centrifugal filter (CF) (regenerated cellulose) using a pipette. 1.5 mL of Ethanol was added and the solution was gently mixed by inversion of the CF (three times). The solution was incubated for 5 minutes and then centrifuged at 5200 rpm for 15 min. After centrifugation the volume of the filtrate should be at least 10 mL (for 100 k filter) or 12 mL (for 30K filter). Subsequently, the sample was diluted immediately with DI water to a final volume of 15 mL and centrifuged at 5200 rpm 15 min. This step was done twice. The entire process starting with the addition of 1.5 mL of ethanol above through the two centrifugations with DI water was repeated once. Further water washing/centrifugation may be optionally used until the filtrate no longer contained free ICG. The UV-vis-NIR spectrum of the final polymersome after purification is shown in FIG. 49.

### Example 4: Method to Make ICGJ Loaded Polymersomes without a PEI Coating on the ICGJ

The procedure in this example describes polymersomes made without any PEI. In this case ICG J-aggregates were added to the outer W2 phase as a sacrificial surfactant such that ICG that goes to the water-oil interface to aid the stability of the ICGJ in the final polymersomes. The polymersomes were synthesized in the same way as in Example 3 except that the ICGJ, in the W1 phase, is not coated with PEI, and modifications were made in the purification.

After evaporation to form the polymersomes, the excess PEG-PLGA and PVA were removed by centrifuging with in DI water in 15 mL tube at 5000 rpm for 3 minutes. A method to purify Ps with a 30 kDa pore size filter was developed by modification of the method described in Example 3 above. In this case, the concentration of ICG and ICGJ not in the Ps was lowered markedly by adaptation of the method in Example 3 as follows: 1) Incubation with ethanol (10%) for 5 minutes and centrifugation at 5200 rpm for 21 minutes with the 30 K filters (2 times); 2) Washing with DI water 3 times with 30K filters via centrifugation at 5200 rpm for minutes; and 3) Collection and dilution of the supernatant with DI to a total volume of 10 mL.

For TEM preparation, samples were placed on 100 mesh carbon coated, formvar coated copper grids treated with poly-1-lysine for approximately 1 hour. Samples were then negatively stained with Millipore-filtered aqueous 1% uranyl acetate for 1 minute. Stain was blotted dry from the grids with filter paper and samples were allowed to dry. Samples were then examined in a JEM 1010 transmission electron microscope (JEOL, USA, Inc., Peabody, MA) at an accelerating voltage of 80 Kv. Digital images were obtained using the AMT Imaging System (Advanced Microscopy Techniques Corp., Danvers, MA).

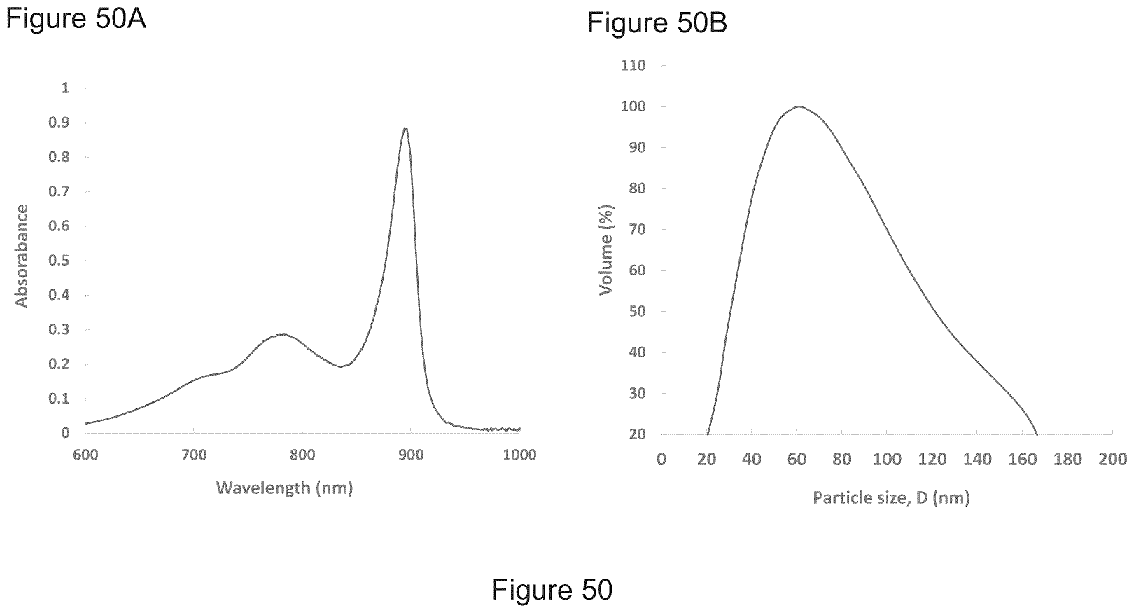

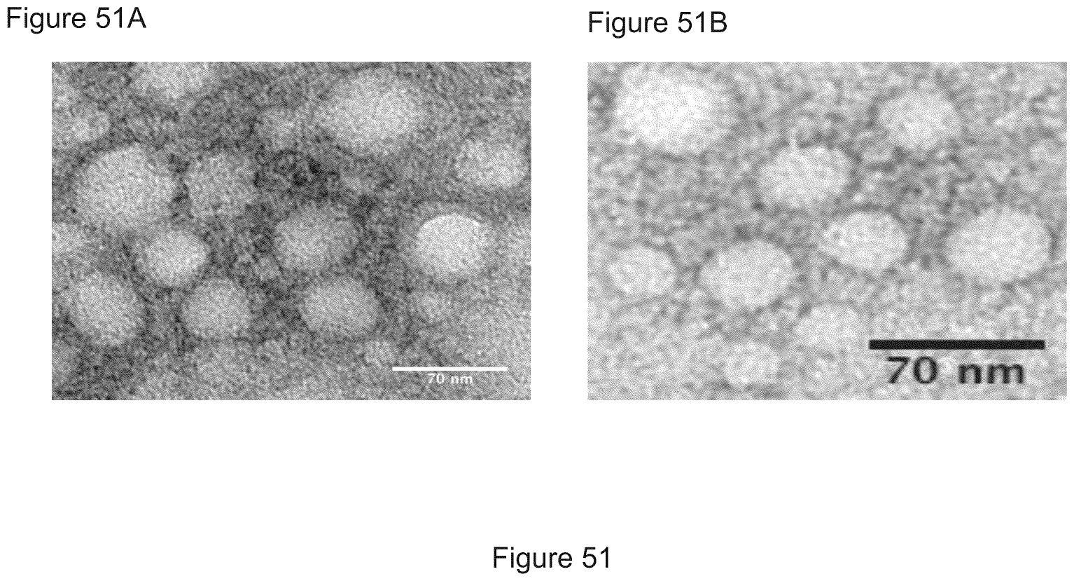

After purifying the Ps, the UV-vis-NIR spectrum and the hydrodynamic diameter of the as-purified polymersomes were determined as shown in FIG. 50A and FIG. 50B, respectively. The volume-averaged hydrodynamic diameter is 70-80 nm from dynamic light scattering. TEM was obtained after three weeks. After three weeks, the sub-100 nm polymersomes were characterized by TEM as shown in FIG. 51A and FIG. 51B. The sizes were comparable to the DLS results. Examples of sub-100 nmr Ps were evident in the TEM micrographs. Some smaller objects were present that may indicate some degradation of the Ps or they may be unencapsulated ICGJ.

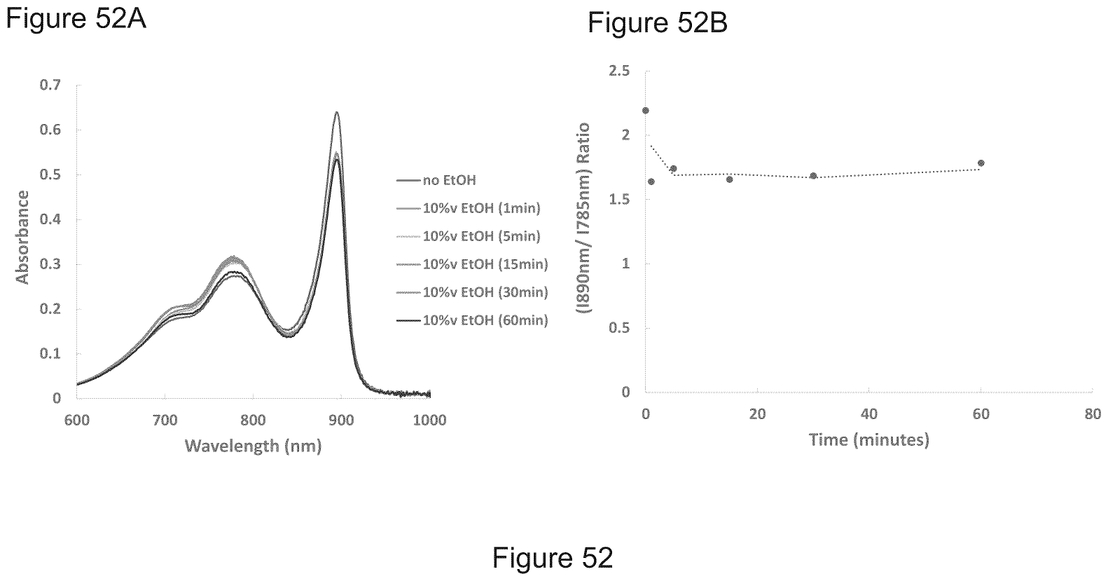

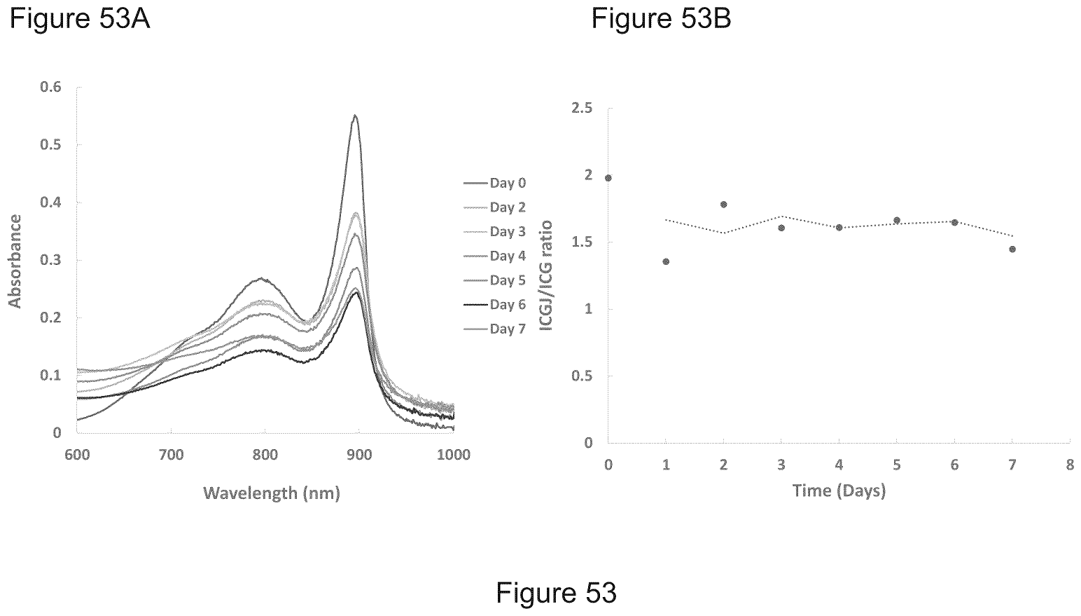

The polymersomes were exposed to 10% ethanol and 100% FBS (FIG. 52 and FIG. 53, respectively) to determine the stability. In both cases, there was a small decrease in the ICGJ peak compared to the initial condition that may have been associated with some free ICGJ not in the polymersomes. After that, the ICGJ peak stayed almost constant in both solutions, 10% ethanol and 100% FBS for more than 60 minutes and more than 6 days, respectively. In 100% FBS, ICGJ aggregates dissociate rapidly. Thus, the small amount of dissociation in FIG. 53 indicates that the ICGJ was protected from this dissociation by the polymer, as it was encapsulated in a nanocapsule. The morphology of the nanocapsule may be expected to be Ps.

### Example 5: Purification of ICGJ@PEI with Filtration Instead of the Ultracentrifuge

In the purification, it is necessary to separate ICG monomer outside the capsules from the ICGJ@PEI encapsulated in the Ps. It is also necessary to remove impurities, such as excess PEG-PLGA and PVA. Thus, a method to purify ICGJ@PEI Ps with a 30 kDa filter was developed: 1) Incubation with Ethanol (10%) for 5 minutes and centrifuge at 5200 rpm for 15 minutes with CF 30K (3 times); 2) Wash with DI 5 times with CF 30K and centrifuge at 5200 rpm for 15 minutes; and 3) The supernatant was collected and the residue washed with 200 μL DI and then re-dispersed in 500 μL DI.

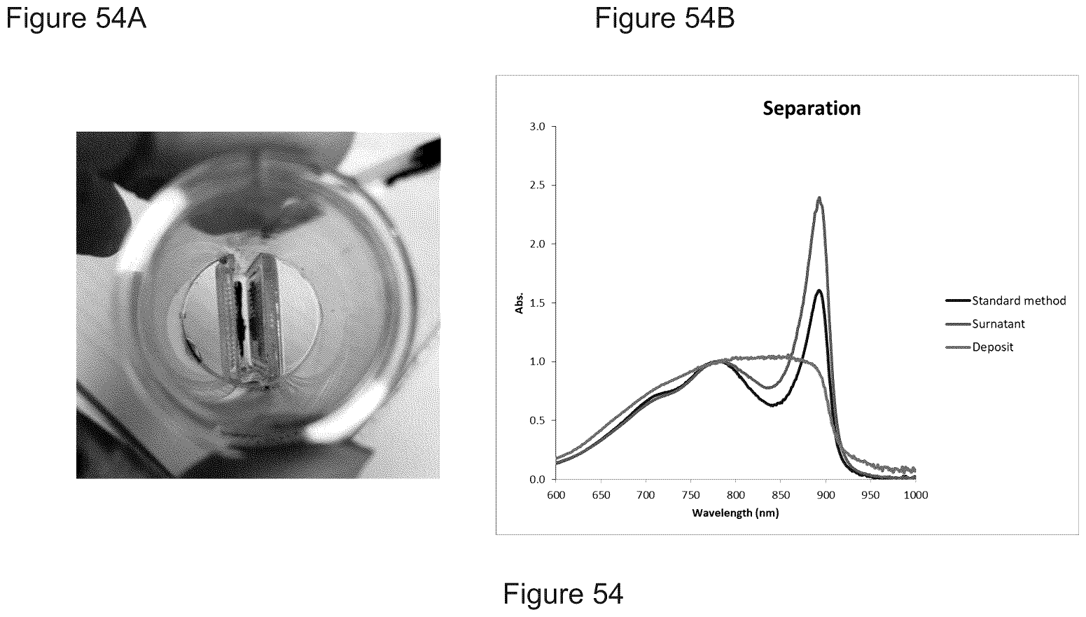

As shown in FIG. 54A, during centrifugation with CF some ICG/partially destroyed Ps deposits at the bottom of the filter tube. As shown in FIG. 54B, in the standard method, where the supernatant and deposit are mixed after filtration in the retentate gives the blue line, which is comparable to the standard condition in Example 1. However, if the supernatant in the herein described method is collected without the deposit, I₈₉₅/I₇₈₀ increases to 2.4. Without ultracentrifuge but using the 30 K or 300 K D filter (3 nm (100 K is about 10 nm)), it is likely that any free PLGA-PEG does not pass through the filter.

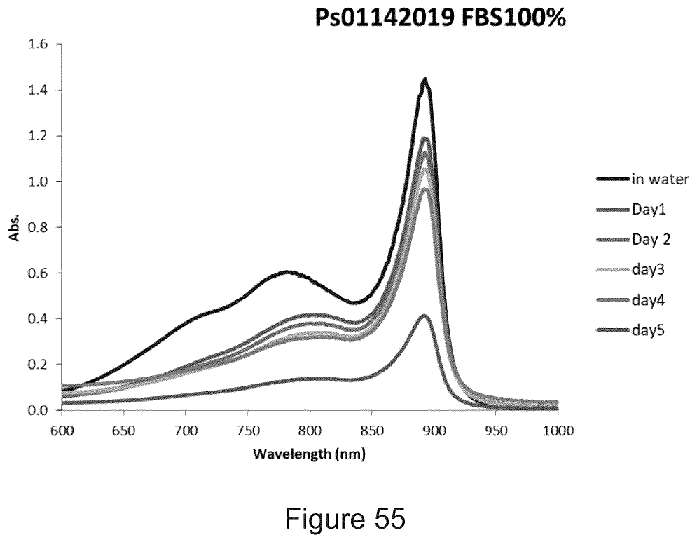

The result for the Ps1142019 sample stability in pure FBS solution is shown in FIG. 55A. Ps1142019 is very stable for at least 4 days in FBS. Therefore, the ICGJ must be encapsulated or otherwise it would have dissociated.

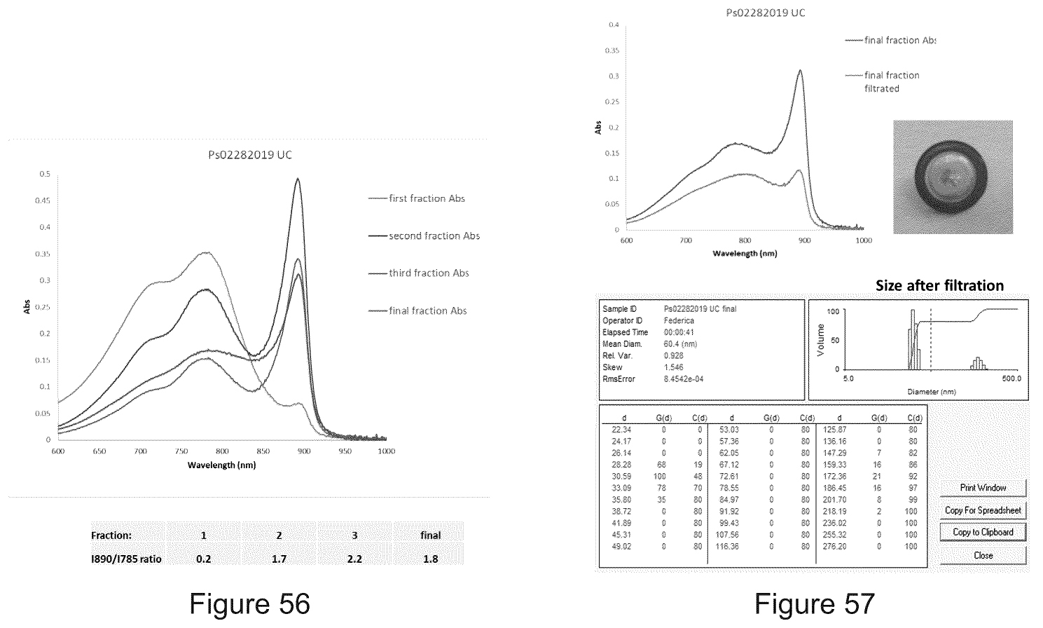

Furthermore, as shown in Table 11, the herein described purification yielded similar properties as the ultracentrifuge purification step described in Example 1 (Table 1). Example 5 was performed with a filter. ICG concentration was measured to get yields using the ICG concentration in supernatant of the first three fractions and then so called final one is for the pellet at bottom after three centrifugations in series. It is likely the yield is higher with the centrifugal filtration method as shown in FIG. 56 and FIG. 57.

TABLE 11
Properties from Ps purified with CF 30K-keeping everything in CF
(Example 5) vs the ultracentrifuge method (Example 1). Free polymer
is in there in Example 5.

| Average Properties Based on Optimized | | |
|---|---|---|
| Experimental Parameters Including | Example | Example |
| Standard Deviation | 1 | 5 |
| ICGJ@PEI hydrodynamic diameter | 56 ± 5 | 52 |
| Polymersome I₈₉₀/I₇₈₅ | 1.6 ± 0.1 | 1.6 |
| Polymersome vol avg. hydrodynamic diameter [nm] | 77 ± 8 | 76 |
| Encapsulation efficiency [%] | 27 ± 4 | 32 |
| Polymer recovery [%] | 39 ± 5% | 47 |
| ICG/Polymer [w/w] | 0.7 ± 0.1 | 0.7 |

PVA remaining after centrifugal filtration was also investigated. This method employed 1) Empty Ps were centrifuged 6 times with CF 30K or CF 100K at 5200 rpm for 15 minutes (DV=295); and 2) The retentate was collected in 1.5 mL of DI. As a control, standard solution of PVA at 1.7 mg/mL (same concentration in Ps) was centrifuged with the same method as for Ps, using 30K or 100K CF. DI filtered 6 times with CF 30K or 100K was used as a blank. Results shown in Table 12 and Table 13 were obtained using TOC analysis.

TABLE 12
% PVA remaining after filtration.

| | CF 30K | CF 100K |
|---|---|---|
| PVA | 0.8% | <dl |

TABLE 13
PEG-PLGA Recovery (%) showing excess of PEG-PLGA in the sample.

| Method | Example 1 | CF 30K | CF 100K |
|---|---|---|---|
| CF only | 39 ± 5% | 81% | 67% |

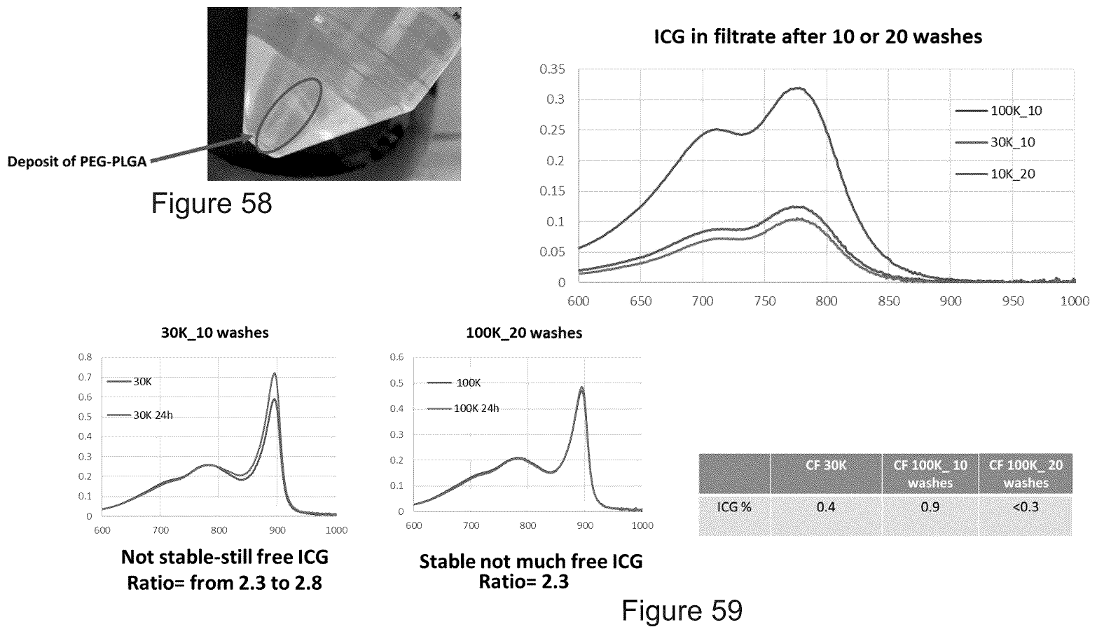

Removal of excess of PEG-PLGA by centrifugation was investigated next using excess of PEG-PLGA that is not soluble in water and can be removed by centrifugation at 5000 rpm for 3 minutes (FIG. 58; Y-shaped material near center). As shown in Table 13, the excess of PEG-PLGA can be removed by centrifugation. Some of the PEG-PLGA is also filtrated/lost during CF with 100K more than 30K.

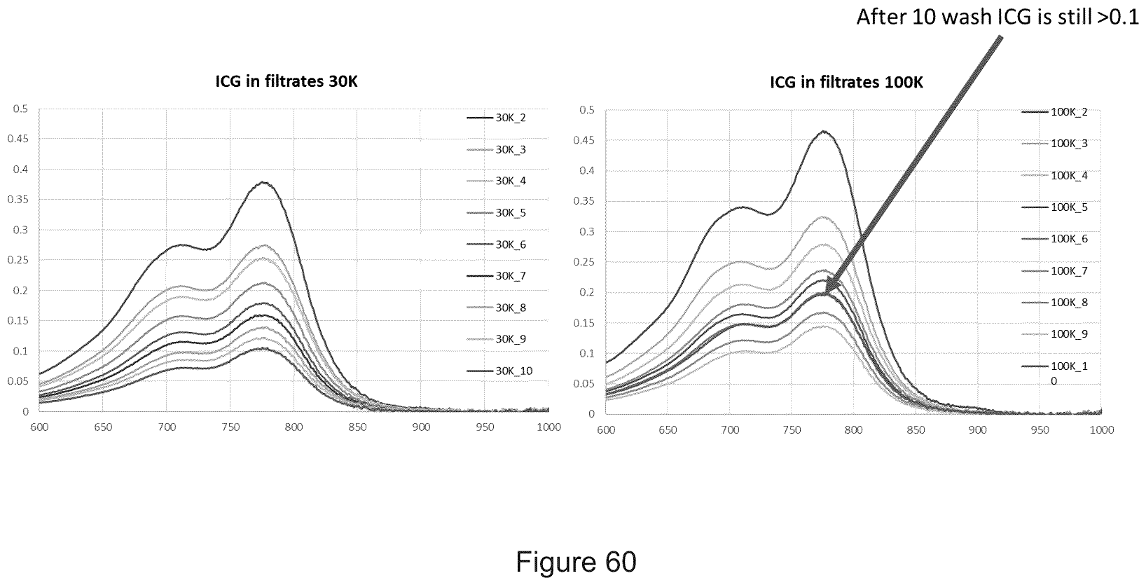

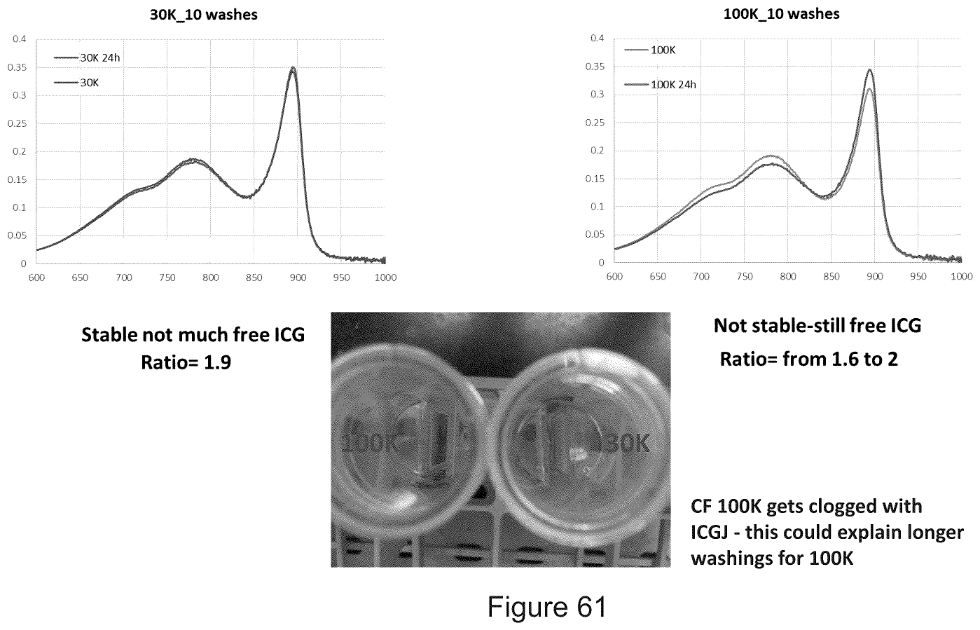

Purification method was also modified to: 1) Incubation with Ethanol (10%) 5 minutes and centrifuge at 5200 rpm for 15 minutes with CF 30K or 100K (4 mL Ps+9.5 mL DI+1.5 mL Ethanol); 2) Wash with DI at 5200 rpm for 15 minutes (this step was repeated twice because the volume of the 100K did not drop after 15 minutes centrifugation); 3) Repeat step 1 and 2; 4) Wash with DI and centrifuge until Abs <0.1 at 5200 rpm for 15 min; and 5) Collect the sample in 3 mL DI and check using UV-vis-NIR (FIG. 60 and FIG. 61).

In summary, a method for washing may be as follows: 1) Centrifuge at 5000 rpm for 3 minutes to remove excess PEG-PLGA; 2) Proceed with Ethanol incubation using lower amount of Ps (4 mL instead of 5 mL); 3A) Use CF 100K (to remove PVA) or 3B) Use CF30K (if it is desirable to keep the 0.8% PVA); 4) Wash with DI water twice; 5) Repeat step 2; 6) Wash with DI water until Abs of filtrate <0.1 (corresponding to ICG <0.3% of the final sample). Experiments to change CF100K after the ethanol step are also performed.

### Example 6: Method to Make ICGJ Loaded Polymersomes with a Higher I₈₉₀/I₇₈₅ Ratio by Heating Polymersomes to 65° C.

The present example discloses polymersomes made without any PEI. The polymersomes were synthesized using the procedure disclosed in Example 4 above except that the ICGJ, in the W1 phase, was not coated with PEI.

After evaporation to form the polymersomes, the excess PEG-PLGA and PVA were removed by centrifuging with in DI water in 15 mL tube at 5000 rpm for 3 minutes. A method to purify Ps with a 30 kDa pore size filter was developed by modification of the method described in Example 3 above. In this case, the concentration of ICG and ICGJ not in the Ps was lowered markedly by adaptation of the method described in Example 3 as follows:
1) incubating with EtOH (10%) for 5 minutes and centrifuging at 5200 rpm for 21 minutes with the 30 K filters (2 times) with one DI water wash for 15 minutes at 5200 rpm in between;
2) washing with DI water 4 times with 30K filters via centrifugation at 5200 rpm for 15 minutes; and
3) collecting and diluting the supernatant with DI to a total volume of 10 mL. After that, the polymersomes samples were aged in the oven at 65° C. for 24 hours to form additional J-aggregates from ICG in the system.

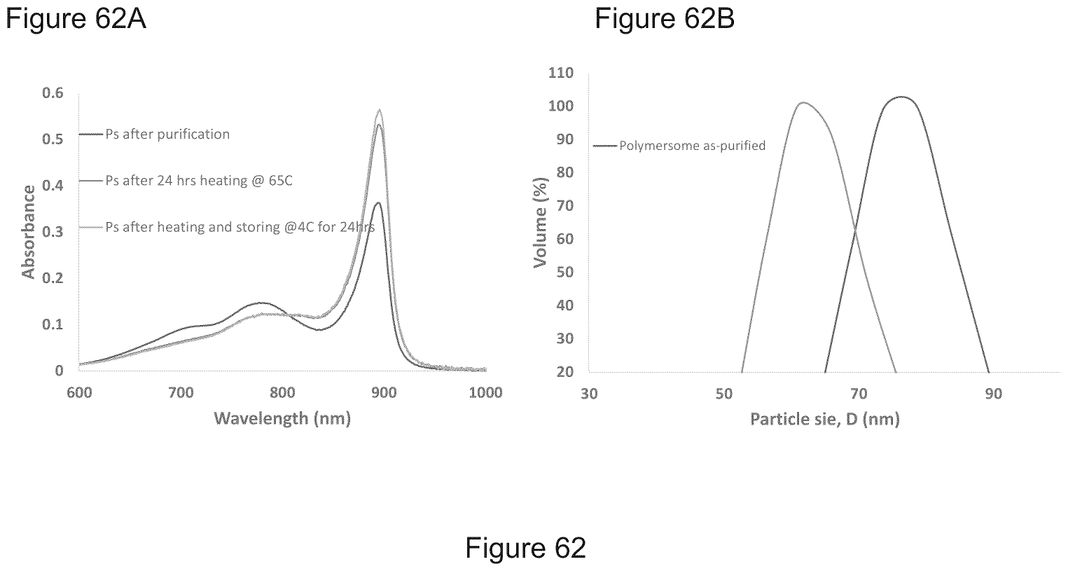

The UV-vis spectra and the hydrodynamic diameter of the as-purified and heated polymersomes were then determined as shown in FIG. 62A and FIG. 62B, respectively. The volume-averaged hydrodynamic diameter was 80 nm from dynamic light scattering. The Zeta potential of the as-heated and as-purified polymersomes was obtained as shown in Table 14. The increase in the magnitude of the Zeta potential after heating indicated that some of the formed J-aggregates by heating were outside the polymersomes core as ICG monomer was both W1 and W2 phases. In addition, the polymersome I₈₉₀/I₇₈₅ ratio was monitored for days to check its stability as shown in Table 14 below.

TABLE 14
I₈₉₀/I₇₈₅ ratio stability of polymersomes together with
zeta potential obtained using Brookhaven Zeta PALS.

| | | | I₈₉₀/I₇₈₅ ratio | I₈₉₀/I₇₈₅ ratio | | Zeta |
|---|---|---|---|---|---|---|
| | | I₈₉₀/I₇₈₅ ratio | after storage @ | after storage @ | Zeta | Potential |
| | I₈₉₀/I₇₈₅ ratio | after heating for | 4° C. for | 4° C. for | Potential | After 24 hours |
| Sample # | as-purified | 24 hours | 24 hours | 11 days. | as purified | heating |
| 1 | 2.4 | 3.4 | 3.5 | 5.2 | −22.18 mV | −39.7 mV |
| 2 | 2.4 | 3.9 | 4.2 | 5.5 | −19.56 mV | −39.9 mV |

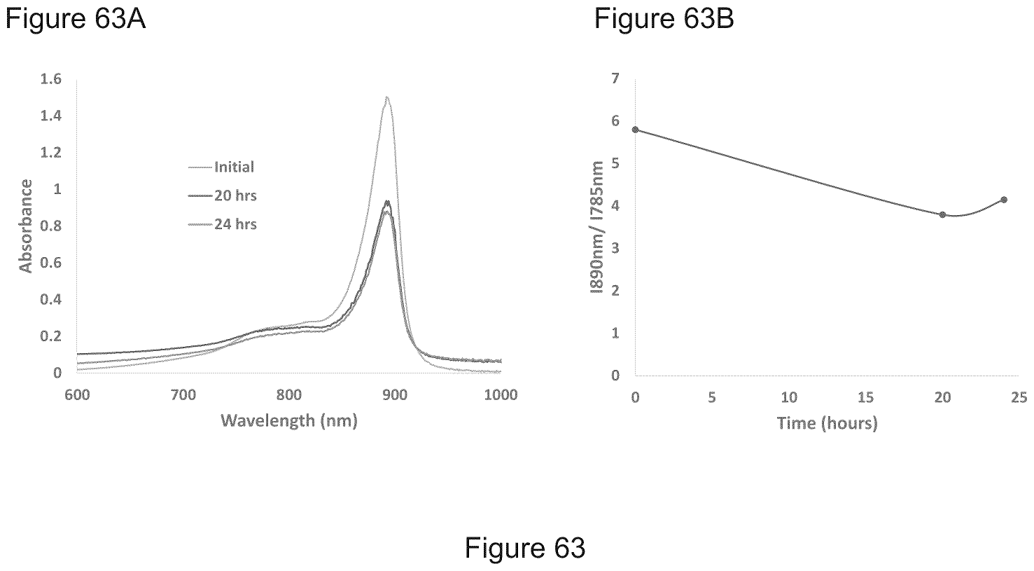

The polymersomes (0.2 mL of 0.2 mg/mL ICG concentration) were exposed to 0.8 mL of 100% FBS, which gave a final FBS concentration of 80% and 0.04 mg/mL final ICG concentration of Ps (FIG. 63). There was an initial decrease in the ICGJ peak compared to the initial condition that is likely to be associated with some free ICGJ not in the polymersomes. After that, the ICGJ peak stayed almost constant as reported previously.

### Example 7: Method to Make ICGJ Loaded Polymersomes at Higher I₈₉₀/I₇₈₅ Ratio by Storing the Polymersomes in Excess PVA

The present example discloses the polymersomes made without any PEI. The polymersomes were synthesized using the procedure disclosed in Example 3 except that the ICGJ, in the W1 phase, was not coated with PEI.

After evaporation to form the polymersomes, the excess PEG-PLGA and PVA were removed by centrifuging with in DI water in 15 mL tube at 5000 rpm for 3 minutes. A method to purify Ps with a 30 kDa pore size filter was developed by modification of Example 3 above. In this case, the concentration of ICG and ICGJ not in the Ps was lowered markedly by adaptation of the method in Example 3 as follows:
1) incubating with EtOH (10%) for 5 minutes and centrifuging at 5200 rpm for 21 minutes with the 30 K filters (2 times) with one DI water wash for 15 minutes at 5200 rpm in between;
2) washing with DI water 4 times with 30K filters via centrifugation at 5200 rpm for 15 minutes; and
3) collecting and diluting the supernatant with DI to a total volume of 10 mL. After that, PVA was added back to the polymersomes samples to stabilize the polymersomes and enhance their I₈₉₀/I₇₈₅ ratio. Sample 1 had 5 wt % PVA while Sample 2 has 2.5 wt % PVA.

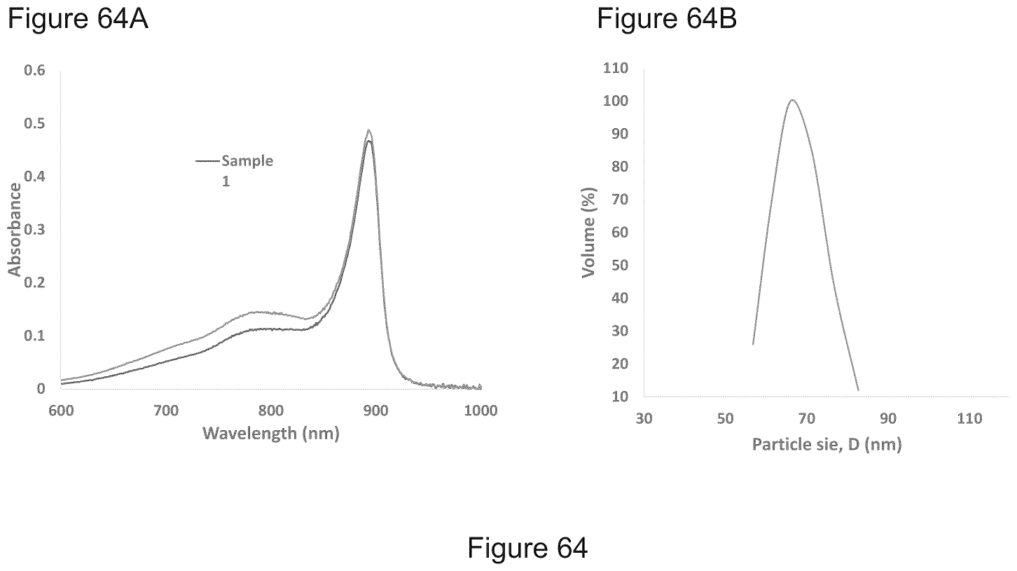

The UV-vis spectra and the hydrodynamic diameter of the as-purified and heated polymersomes were then determined as shown in FIG. 64A and FIG. 64B, respectively. The volume-averaged hydrodynamic diameter is 70-80 nm from dynamic light scattering.

The polymersomes I₈₉₀/I₇₈₅ ratio was monitored for days to check its stability as shown in Table 15 below.

TABLE 15
I₈₉₀/I₇₈₅ ratio stability of polymersomes together with zeta potential
obtained using Brookhaven Zeta PALS.

| | I₈₉₀/I₇₈₅ | I₈₉₀/I₇₈₅ ratio after | I₈₉₀/I₇₈₅ ratio after | Zeta |
|---|---|---|---|---|
| Sample | ratio | storage @ 4° C. | storage @ 4° C. | Potential |
| # | as-purified | for 24 hours | for 48 hours | as purified |
| 1 | 4.0 | 5.8 | 5.91 | −23.6 mV |
| 2 | 3.3 | 5.2 | 6.03 | −24.6 mV |

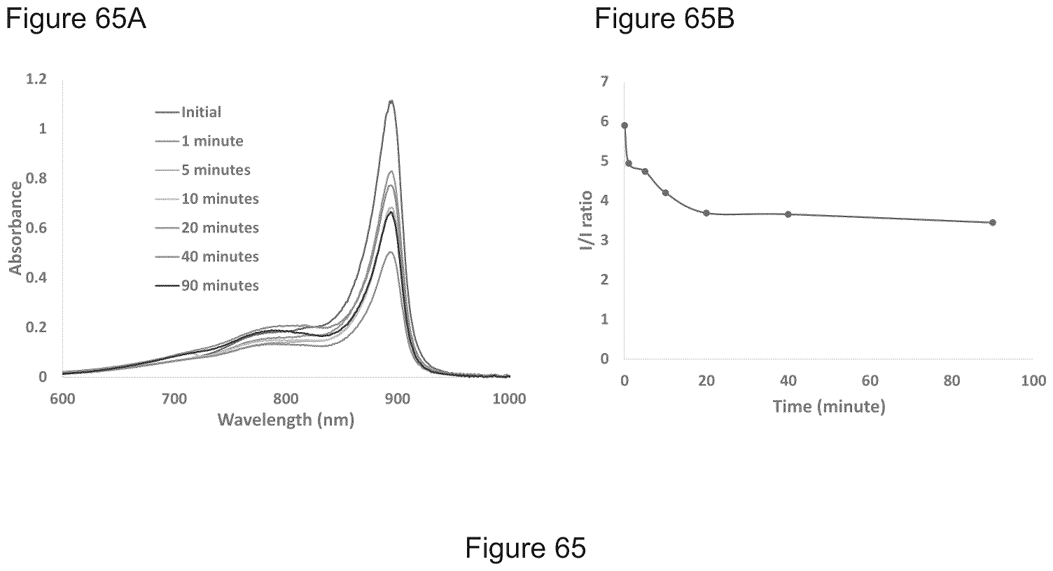

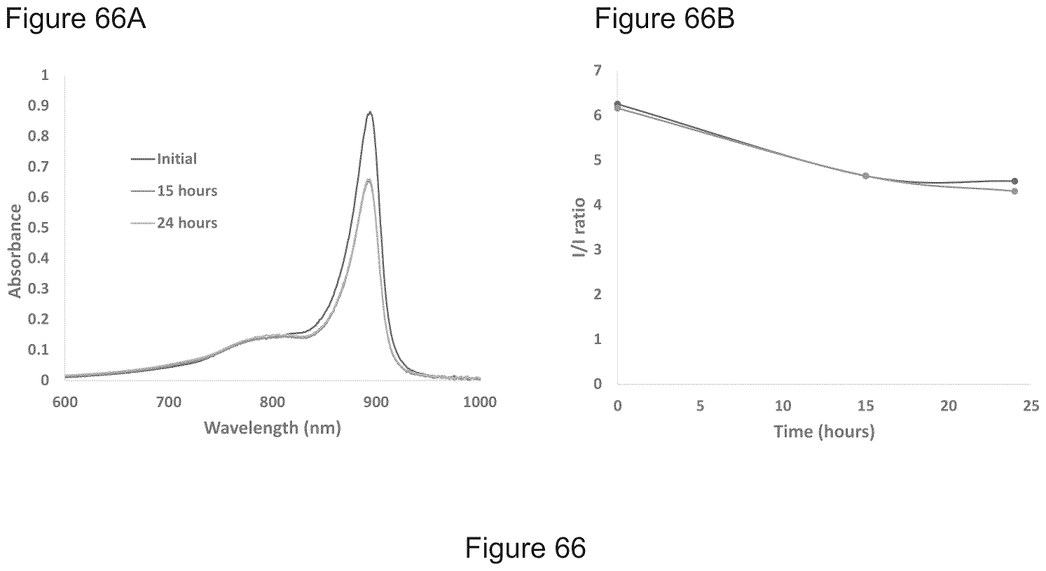

The polymersomes were exposed to 10% ethanol and 80% FBS in a similar way as in Example 5 (FIG. 65 and FIG. 66, respectively) to determine the stability. In both cases, there was a small decrease in the ICGJ peak compared to the initial condition that may have been associated with some free ICGJ not in the polymersomes. After that, the ICGJ peak stayed almost constant in both solutions, 10% ethanol and 80% FBS for more than 60 minutes and 24 hours, respectively.

### Example 9: PEI-Free Polymersomes Detected in the Cells

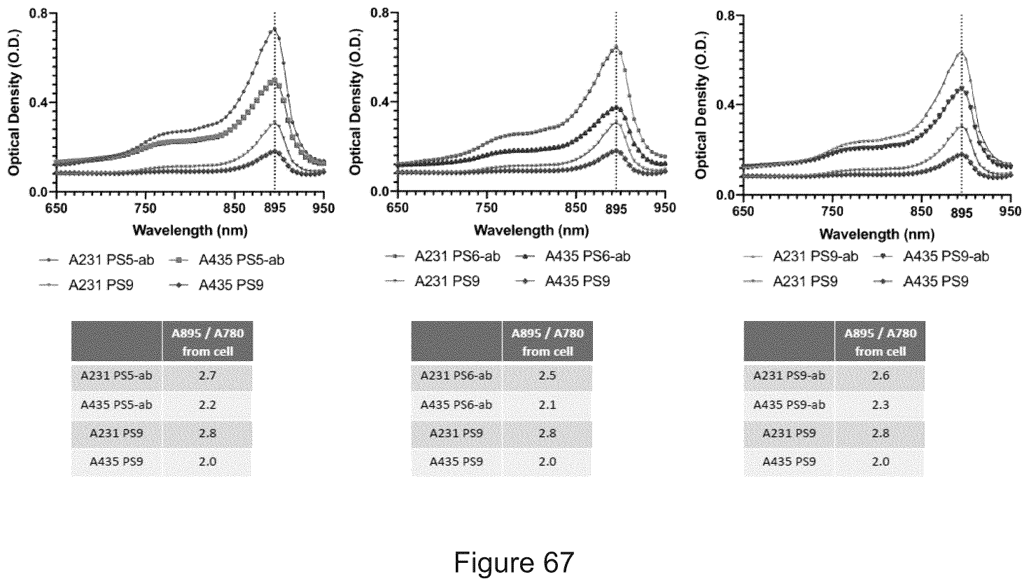

The PS were formed in the same way as in Example 3 but without PEI, as described in Example 4. The behavior is investigated for the same two cell lines as in FIG. 22. The protocols were the same as in the section Cellular Response to ICGJ@PEI Ps and Molecular Specific Photoacoustic Imaging. The experiments used 100 k cells/sample. In each case the absorbance was higher when the antibody was attached to the Ps with click chemistry as shown in Table 16 and FIG. 67. In all cases the intensity ratio for 895 to 780 nm was above 2 indicating ICGJ aggregates were present.

TABLE 16
Increase in absorbance at 895 nm of Ps with grafted antibody
when antibody was added to the polymersome.

| | A231 |
|---|---|
| PS5-ab vs PS-9 @A895 nm | 2.4 |
| PS6-ab vs PS-9@A895 nm | 2.1 |
| PS9-ab vsPS-9@A895 nm | 2.1 |

In Table 17, the cellular uptake was higher in each case for the A231 cells versus A435 cells as was also the case in FIG. 22.

TABLE 17
Absorbance ratio at 895 nm for A231 cells versus A435 cells.

| | PS5-ab | PS6-ab | PS9-ab |
|---|---|---|---|
| A231 vs A435 A895 nm | 1.5 | 1.7 | 1.4 |

### Example 10: Glossary

TABLE 18
Glossary of terms and abbreviations used herein.

| | | |
|---|---|---|
| Subscript | | |
| | c | Capsule |
| | P | PLGA based polymer |
| | I | ICG (without PEI) |
| | IJ | ICGJ (without PEI) |
| Superscript | | |
| | 1 | Individual species |
| | c | Carbon |
| Variables | | |
| | ρ | Density |
| | ρₚ | Density of PEG-PLGA |
| | ρ_{IJ} | Density of ICGJ |
| | Ab | Absorbance |
| | Ab_{I} | Absorbance of ICG@790 nm in DI/Ethanol |
| | Ab_{I}_{^{oo}} | Absorbance of ICG@790 nm in DI |
| | C | Concentration |
| | C°_{I} | Concentration of ICG (monomer) |
| | C_{I} | Concentration of ICG (monomer and J) |
| | C_{P} | Concentration of PEG-PLGA |
| | CF | Carbon fraction |
| | CF_{I} | Carbon fraction of ICG |
| | CF_{P} | Carbon fraction of PEG-PLGA |
| | CF_{PEI} | Carbon fraction of PEI |
| | DF | Dilution factor |
| | m | Mass |
| | M°_{I} | Mass of ICG (monomer) |
| | M_{I} | Mass of ICG (monomer and J) |
| | M^{C}_{I} | Carbon mass of ICG (monomer an J) |
| | M_{IJ} | Mass of ICGJ |
| | Mₚ | Mass of PEG-PLGA |
| | M¹_{IJ} | Mass of one ICGJ |
| | M^{C}_{PEI} | Carbon mass of PEI |
| | M_{PEI} | Mass of PEI |
| | M^{C}_{P} | Carbon mass of PEF-PLGA |
| | N | Number |
| | N_{c} | Number of capsules |
| | N_{IJ} | Number of ICGJ NPs |
| | r | Radius |
| | r₁ | Inner capsule radius |
| | r₂ | Outer capsule radius |
| | V | Volume |
| | V¹_{IJ} | Volume of one ICGJ |
| | V¹ₛ | Volume of polymersome’s shell |
| | Vₛₐₘₚₗₑ | Total polymersome sample volume |

The disclosures of each and every patent, patent application, and publication cited herein are hereby incorporated herein by reference in their entirety. While this invention has been disclosed with reference to specific embodiments, it is apparent that other embodiments and variations of this invention may be devised by others skilled in the art without departing from the true spirit and scope of the invention. The appended claims are intended to be construed to include all such embodiments and equivalent variations.

1. A composition comprising a polymer nanocapsule and a dye, wherein the polymer nanocapsule encapsulates the dye, and
wherein the polymer nanocapsule has an average hydrodynamic diameter of about 50 nm to about 200 nm and comprises a block copolymer selected from the group consisting of poly(ethylene oxide) (PEO) block copolymer, poly(lactide-co-glycolic acid)-block-polyethylene glycol (PLGA-b-PEG), a biodegradable PLGA-b-PEG, poly(ethylene oxide)-block-poly(ε-caprolactone) (PEO-b-PCL), polyanhydride-block-PEG copolymers, zwitterionic poly(carbobetaine) and zwitterionic poly(sulfobetaine)-containing block copolymers, poly(trimethylene carbonate)-block-poly(L-gluatamic acid), poly(ethylene glycol-block-L-aspartic acid), poly(ethylene oxide)-block-polypropylene oxide copolymers, and any combination thereof; and
wherein the dye is a dye aggregate and is selected from the group consisting of a polymethine dye, cyanine dye, hemicyanine dye, streptocyanine dye, mercocyanine dye, oxonol dye, styryl dye, diarylmethine dye, triarylmethine dye, rylene, squaraine, perylene bismide dye or aza-analog thereof, indocyanine green (ICG), Congo Red, IR783, Briliant Blue G, rhodamine 6G, and any combination thereof.

2.-4. (canceled)

5. The composition of claim 1, wherein the dye is an indocyanine green (ICG) aggregate and the polymer nanocapsule comprises a PLGA-b-PEG; wherein the PLGA-b-PEG encapsulates the ICG aggregate.

6. The composition of claim 5, wherein the ICG aggregate is an ICG J aggregate.

7. The composition of claim 1, wherein the composition further comprises a cationic polymer.

8. The composition of claim 7, wherein the cationic polymer is polyethyleneimine (PEI).

9. The composition of claim 1, wherein the composition comprises a targeting domain attached to the surface of the polymer nanocapsule.

10. The composition of claim 9, wherein the targeting domain binds to at least one cancer cell.

11. The composition of claim 1, wherein the composition further comprises a therapeutic agent.

12. A contrast agent comprising the composition of claim 1 and a pharmaceutically acceptable excipient.

13. An imaging method, comprising:
contacting a biological tissue with the composition of claim 1,
applying energy to a biological tissue comprising the composition, and
imaging the biological tissue comprising the composition.

14. The method of claim 13, wherein applying energy to the biological tissue comprises application of at least one selected from the group consisting of: exposing the biological tissue to irradiation at a wavelength between about 680 and 1100 nm; irradiating at least a portion of the biological tissue with a light source; applying a radio frequency field; and any combination thereof.

15. The method of claim 13, wherein imaging the biological tissue comprises application of at least one imaging technique selected from the group consisting of: photoacoustic imaging, ultrasound imaging, optical imaging, magnetic resonance imaging, computed tomography, thermal imaging, nuclear imaging, magnetomotive imaging enhancement, and any combination thereof.

16. The method of claim 13, wherein imaging the biological tissue comprises transducing the resulting ultrasound signal from the biological tissue and producing an image in a data processor from the transduced ultrasound signal.

17. The method of claim 13, wherein the composition further comprises at least one therapeutic agent.

18. The method of claim 13, wherein the composition further comprises a targeting domain.

19. The method of claim 18, further comprising:
allowing the composition to accumulate in a region of the biological tissue,
wherein the targeting domain facilitated accumulation of the composition in the region.

20. A method of treating a disease or disorder in a subject in need thereof, the method comprising administering a therapeutically effective amount of the composition of claim 1 to the subject.
